(19) United States
(12) Patent Application Publication
(10) Pub. No.: US 2024/0251661 A1
(43) Pub. Date: Jul. 25, 2024

(54) ORGANIC LIGHT EMITTING ELEMENT AND DISPLAY DEVICE

(71) Applicant: LG Display Co., Ltd., Seoul (KR)

(72) Inventors: Ji Cheol SHIN, Gyeongsangnam-do (KR); Seonkeun YOO, Gyeonggi-do (KR); Su Bin AHN, Gyeonggi-do (KR)

(73) Assignee: LG Display Co., Ltd., Seoul (KR)

(21) Appl. No.: 18/531,337

(22) Filed: Dec. 6, 2023

(30) Foreign Application Priority Data
Dec. 31, 2022 (KR) 10-2022-0191366

(51) Int. Cl. H10K 85/60; C07C 255/51; C07D 213/84; C07D 471/04; C09K 11/06

(52) U.S. Cl. CPC H10K 85/615; C07C 255/51; C07D 213/84; C07D 471/04; C09K 11/06; H10K 85/654; H10K 50/131

(57) ABSTRACT

An organic light emitting element and a display device, specifically, an organic light emitting element including a first compound represented by chemical formula 1 and a second compound represented by chemical formula 2 provides excellent efficiency, long lifespan or low driving voltage and a display device including the organic light emitting element.

### CROSS-REFERENCE TO RELATED APPLICATION

This application claims priority from Korean Patent Application No. 10-2022-0191366, filed on Dec. 31, 2022, which is hereby incorporated by reference for all purposes as if fully set forth herein.

### BACKGROUND

### Field

Embodiments of the disclosure relate to an organic light emitting element and a display device.

### Description of Related Art

In general, organic light emission refers to a phenomenon in which electric energy is converted into light energy by an organic material. The organic light emitting element refers to a light emitting element using the organic light emission phenomenon. The organic light emitting element has a structure including an anode, a cathode, and an organic material layer disposed therebetween.

The organic material layer may have a multilayer structure composed of different materials to increase the efficiency and stability of the organic light emitting element and may include a light emitting layer (also referred to as an emission material layer (EML)).

The lifespan and efficiency are the most important issues with organic light emitting elements. The efficiency, lifespan, and driving voltage are related to each other. If the efficiency is increased, the driving voltage is relatively decreased, so that the crystallization of the organic material by the Joule heating during driving is reduced, leading to an increase in lifespan.

The role of the light emitting layer EML is important to enhance the light emitting properties of the organic light emitting element and increase the lifespan. In particular, to have high-efficiency characteristics, the host material of the light emitting layer desirably has a high triplet energy level, and further a sufficient stability of the material is needed.

### BRIEF SUMMARY

An organic light emitting element may include an organic material layer including a hole injection layer and a charge generation layer between an anode and a cathode. The hole injection layer and the charge generation layer are layers closely related to hole injection and movement characteristics that determine the characteristics of the device, and organic electron acceptor compounds may be used for efficient hole generation, injection and movement. Since the organic electron acceptor compound includes a strong electron withdrawing group (EWG), when the hole injection layer is doped with it, it may withdraw electrons from a high occupied molecular orbital (HOMO) energy level of the adjacent hole transport layer to a low occupied molecular orbital (LUMO) energy level of the organic electron acceptor compound to generate holes and inject the holes into the hole transport layer. Therefore, organic electron acceptor compounds may be designed to include a number of strong electron withdrawing groups for efficient hole generation, injection and transfer.

Organic electron acceptor compounds may contain many strong electron withdrawing groups having low LUMO energy levels. However, since the organic electron acceptor compounds typically have a low miscibility with the hole transporting compound, a high driving voltage and low luminous efficiency may occur due to inefficient charge injection and transfer characteristics. Accordingly, the inventors of the disclosure have invented an organic light emitting element and a display device that may have high efficiency, long lifespan or low driving voltage.

Embodiments of the disclosure may provide an organic light emitting element and a display device that may have high efficiency, long lifespan, and/or low driving voltage.

Embodiments of the disclosure may provide an organic light emitting element including a first electrode, a second electrode, and an organic material layer positioned between the first electrode and the second electrode.

The organic material layer may include a first compound represented by chemical formula 1 described above.

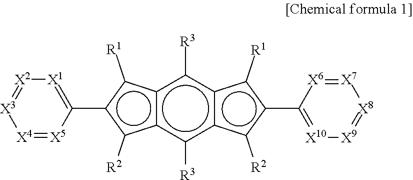

The organic material layer may include a second compound represented by chemical formula 2 described below.

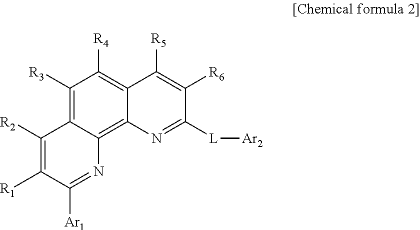

Embodiments of the disclosure may provide a display device including the organic light emitting element described above.

### EFFECTS OF THE DISCLOSURE

According to embodiments of the disclosure, there may be provided an organic light emitting element having high emission efficiency, long lifespan, and/or low driving voltage.

According to embodiments of the disclosure, there may be provided an organic light emitting element having high emission eficiency, long lifespan and/or low driving voltage by including a layer having excellent hole injection characteristics or electron injection characteristics.

### DESCRIPTION OF DRAWINGS

The above and other objects, features, and advantages of the disclosure will be more clearly understood from the following detailed description, taken in conjunction with the accompanying drawings, in which:

FIG. 1 illustrates a system configuration of a display device according to embodiments of the disclosure;

FIG. 2 illustrates a subpixel circuit of a display device according to embodiments of the disclosure; and

FIGS. 3, 4, and 5 are cross-sectional views schematically illustrating an organic light emitting element according to embodiments of the disclosure.

### DETAILED DESCRIPTION

In the following description of examples or embodiments of the disclosure, reference will be made to the accompanying drawings in which, by way of illustration, specific examples or embodiments are shown that can be implemented by a person skilled in the art, and in which the same reference numerals and signs can be used to designate the same or like components even when they are shown in different of the accompanying drawings. Further, in the following description of examples or embodiments of the disclosure, detailed descriptions of well-known functions and components incorporated herein will be omitted when it is determined that the description of the same may render subject matter described in conjunction with the embodiments of the disclosure less clear. The terms such as “including,” “having,” “containing,” “constituting” “make up of,” and “formed of” used herein are generally intended to allow other components to be added unless the terms are used with the term “only”. As used herein, singular forms are intended to include plural forms unless the context clearly indicates otherwise.

Terms, such as “first,” “second,” “A,” “B,” “(A),” or “(B)” may be used herein to describe elements of the disclosure. Each of these terms is not used to define essence, order, sequence, or number of elements etc., but is used merely to distinguish the corresponding element from other elements.

When it is mentioned that a first element “is connected or coupled to,” “contacts or overlaps” etc. a second element, it should be interpreted that, not only can the first element “be directly connected or coupled to” or “directly contact or overlap” the second element, but a third element can also be “interposed” between the first and second elements, or the first and second elements can “be connected or coupled to”, “contact or overlap”, etc. each other via a fourth element. Here, the second element may be included in at least one of two or more elements that “are connected or coupled to”, “contact or overlap”, etc. each other.

When time relative terms, such as “after,” “subsequent to,” “next,” “before,” and the like, are used to describe processes or operations of elements or configurations, or flows or steps in operating, processing, manufacturing methods, these terms may be used to describe non-consecutive or non-sequential processes or operations unless the term “directly” or “immediately” is used together with the said relative terms.

In addition, when any dimensions, relative sizes etc. are mentioned, it should be considered that numerical values for an elements or features, or corresponding information (e.g., level, range, etc.) include a tolerance or error range that may be caused by various factors (e.g., process factors, internal or external impact, noise, etc.) even when a relevant description is not specified. Further, the term “may” fully encompasses all the meanings of the term “can”.

Hereinafter, various embodiments of the disclosure are described in detail with reference to the accompanying drawings.

As used herein, the term “halo” or “halogen” includes fluorine (F), chlorine (Cl), bromine (Br), and iodine (I), and the like, unless otherwise specified.

As used herein, the term “alkyl” or “alkyl group” may mean a radical of a saturated aliphatic functional group having 1 to 60 carbon atoms linked by a single bond and including a straight chain alkyl group, branched chain alkyl group, cycloalkyl (alicyclic) group, alkyl-substituted cycloalkyl group, or cycloalkyl-substituted alkyl group, unless otherwise specified.

As used herein, the term “haloalkyl group” or “halogenalkyl group” may mean a halogen-substituted alkyl group unless otherwise specified.

As used herein, the term “alkenyl” or “alkynyl” may have a double bond or a triple bond, respectively, and may include a straight or branched chain group and may have 2 to 60 carbon atoms unless otherwise specified.

As used herein, the term “cycloalkyl” may refer to an alkyl forming a ring having 3 to 60 carbon atoms, unless otherwise specified.

As used herein, the term “alkoxy group” or “alkyloxy group” refers to an alkyl group to which an oxygen radical is bonded, and may have 1 to 60 carbon atoms unless otherwise specified.

As used herein, the term “alkenoxyl group,” “alkenoxy group,” “alkenyloxyl group,” or “alkenyloxy group” refers to an alkenyl group to which an oxygen radical is attached, and may have 2 to 60 carbon atoms unless otherwise specified.

As used herein, the terms “aryl group” and “arylene group” each refer to a group that may have 6 to 60 carbon atoms unless otherwise specified, but are not limited thereto. In the disclosure, the aryl group or the arylene group may include a monocyclic type, a ring assembly, a fused polycyclic system, a spiro compound, and the like. For example, the aryl group includes, but is not limited to, phenyl, biphenyl, naphthyl, anthryl, indenyl, phenanthryl, triphenylenyl, pyrenyl, peryleneyl, chrysenyl, naphthacenyl, or fluoranthenyl. The term “naphthyl” may relate to 1-naphthyl and 2-naphthyl, and the term “anthryl” may relate 1-anthryl, 2-anthryl and 9-anthryl.

In the disclosure, the term “fluorenyl group” or “fluorenylene group” may refer to a monovalent or divalent functional group, respectively, of fluorene, unless otherwise specified. The “fluorenyl group” or “fluorenylene group” may mean a substituted fluorenyl group or a substituted fluorenylene group. “Substituted fluorenyl group” or “substituted fluorenylene group” may refer to a monovalent or divalent functional group of substituted fluorene. “Substituted fluorene” may mean that at least one of the following substituents R, R′, R″ and R′″ is a functional group other than hydrogen. It may include a situation where R and R′ are bonded to each other to form a spiro compound together with the carbon to which they are bonded.

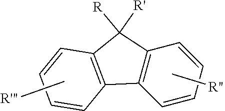

As used herein, the term “spiro compound” refers to a compound that has a ‘spiro union’, and the term “spiro union” means a union formed from two rings that share only one atom. In this case, the atom shared by the two rings may be referred to as a ‘spiro atom’.

As used herein, the term “heterocyclic group” may include not only an aromatic ring, such as a “heteroaryl group” or “heteroarylene group” but also a non-aromatic ring and, unless otherwise specified, means a ring with 2 to 50 carbon atoms and one or more heteroatoms, but is not limited thereto. As used herein, the term “heteroatom” refers to N, O, S, P or Si unless otherwise specified, and the term “heterocyclic” group may designate a monocyclic group containing a heteroatom, a ring assembly, a fused polycyclic system, or a spiro compound.

The “heterocyclic group” may include a ring containing SO2 instead of carbon forming the ring. For example, the “heterocyclic group” may include the following compounds.

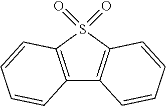 [SMILES: O=S1(=O)c2ccccc2-c2ccccc21]

As used herein, the term “ring” may include monocycles and polycycles, may include hydrocarbon rings as well as heterocycles containing at least one heteroatom, or may include aromatic and non-aromatic rings.

As used herein, the term “polycycle” may include ring assemblies, fused polycyclic systems, and/or spiro compounds, may include aromatic as well as non-aromatic compounds, and/or may include heterocycles containing at least one heteroatom as well as hydrocarbon rings.

As used herein, the term “aliphatic ring group” refers to a cyclic hydrocarbon other than the aromatic hydrocarbon, may include a monocyclic type, a ring assembly, a fused polycyclic system, and a spiro compound and, unless otherwise specified, may designate a ring having 3 to 60 carbon atoms. For example, a fusion of benzene, which is an aromatic ring, and cyclohexane, which is a non-aromatic ring, also corresponds to an aliphatic ring.

As used herein, the term “alkylsilyl group” may refer to a monovalent substituent in which three alkyl groups are bonded to a Si atom.

As used herein, the term “arylsilyl group” may refer to a monovalent substituent in which three aryl groups are bonded to a Si atom.

As used herein, the term “alkylarylsilyl group” may refer to a monovalent substituent in which one alkyl group and two aryl groups are bonded to a Si atom or two alkyl groups and one aryl group are bonded to the Si atom.

As used herein, the term “ring assembly” means that two or more ring systems (single or fused ring systems) are directly connected to each other through single or double bonds. For example, in the case of an aryl group, a biphenyl group or a terphenyl group may be a ring assembly but is not limited thereto.

As used herein, the term “fused polycyclic system” refers to a type of fused rings sharing at least two atoms. For example, in the case of an aryl group, a naphthalenyl group, a phenanthrenyl group, or a fluorenyl group may be a fused polycyclic system, but is not limited thereto.

When prefixes are named successively, it may mean that the substituents are listed in the order specified first. For example, an arylalkoxy group may mean an alkoxy group substituted with an aryl group, an alkoxycarbonyl group may mean a carbonyl group substituted with an alkoxy group, and an arylcarbonylalkenyl group may mean an alkenyl group substituted with an arylcarbonyl group. The arylcarbonyl group may be a carbonyl group substituted with an aryl group.

Unless otherwise explicitly stated, regarding the terms “substituted” or “unsubstituted” as used herein, “substituted” may mean being substituted with one or more substituents selected from the group consisting of halogen, an amino group, a nitrile group, a nitro group, a C₁-C₂₀ alkyl group, a C₁-C₂₀ alkoxy group, a C₁-C₂₀ alkylamine group, a C₁-C₂₀ alkylthiophene group, a C₆-C₂₀ arylthiophene group, a C₂-C₂₀ alkenyl group, a C₂-C₂₀ alkynyl group, a C₃-C₂₀ cycloalkyl group, a C₆-C₂₀ aryl group, a C₈-C₂₀ arylalkenyl group, a silane group, a boron group, a germanium group, and a C₂-C₂₀ heterocyclic group including at least one heteroatom selected from the group consisting of O, N, S, Si and P, but is not limited to these substituents.

In the disclosure, the “functional group names” corresponding to the aryl group, arylene group, and heterocyclic group provided as examples of the symbols and their substituents may be described with the names of the functional groups reflecting the valence, but may also be described with the names of the parent compounds. For example, in the case of “phenanthrene,” which is a type of aryl group, its name may be specified with its group identified, such as “phenanthryl (group)” for the monovalent group, and ‘phenanthrylene (group)’ as the divalent group, but may also be specified as “phenanthrene,” which is the name of the parent compound, regardless of the valence. Similarly, pyrimidine may be specified as “pyrimidine” regardless of the valence or may also be specified as pyrimidinyl (group) for the monovalent group and as pyrimidylene (group) for the divalent group. Therefore, in the disclosure, when the type of the substituent is specified with the name of the parent compound, it may mean an n-valent “group” formed by detachment of the hydrogen atom bonded to a carbon atom and/or a heteroatom of the parent compound.

Further, unless explicitly stated, the formulas used in the disclosure may be applied in the same manner as the definition of the substituent by the exponent definition of the following formulas.

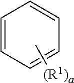

When a is 0, it means that the substituent R¹ does not exist, meaning that hydrogen is bonded to each of the carbon atoms forming the benzene ring. In this case, the chemical formula or chemical compound may be specified without expressing the hydrogen bonded to the carbon. Further, when a is 1, one substituent R¹ is bonded to any one of the carbon atoms forming the benzene ring, and when a is 2 or 3, it may be bonded as follows. When a is an integer of 4 to 6, it is bonded to the carbon of the benzene ring in a similar manner, and when a is an integer of 2 or more, R¹ may be identical or different.

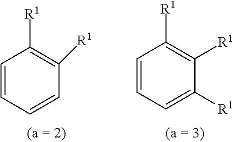

In the disclosure, when substituents are bonded to each other to form a ring, it may mean that adjacent groups are bonded to each other to form a monocycle or fused polycycle, and the monocycle or fused polycycle may include heterocycles containing at least one heteroatom as well as hydrocarbon rings and may include aromatic and non-aromatic rings.

In the disclosure, organic light emitting element may mean a component(s) between an anode and a cathode of an electronic device or an organic light emitting diode including an anode, a cathode, and component(s) positioned therebetween.

In some cases, in the disclosure, organic light emitting element may mean an organic light emitting diode and a panel including the same, or an electronic device including the panel and circuitry. The electronic device may include, e.g., a display device, a lighting device, a solar cell, a portable or mobile terminal (e.g., a smart phone, a tablet, a PDA, an electronic dictionary, or PMP), a navigation terminal, a game device, various TVs, and various computer monitors but, without being limited thereto, may include any type of device including the component(s).

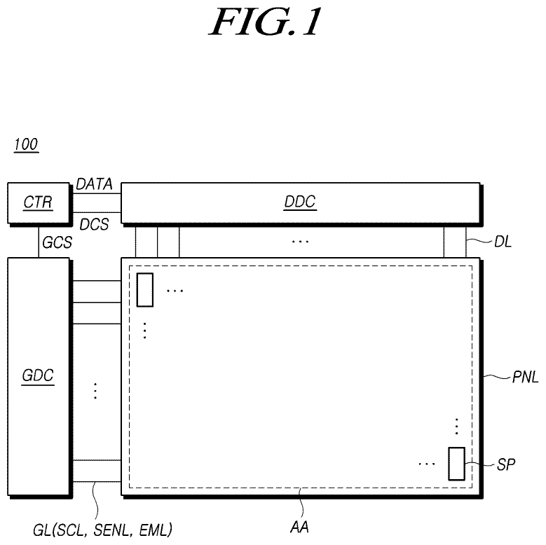

FIG. 1 illustrates a system configuration of a display device 100 according to embodiments of the disclosure.

Referring to FIG. 1, a display device 100 according to embodiments of the disclosure includes a display panel PNL in which a plurality of data lines DL and a plurality of gate lines GL are disposed, and a plurality of subpixels SP defined by the plurality of data lines DL and the plurality of gate lines GL are arranged, a data driving circuit DDC for driving the plurality of data lines DL, a gate driving circuit GDC for driving the plurality of gate lines GL, and a controller CTR for controlling the data driving circuit DDC and the gate driving circuit GDC.

The controller CTR supplies various control signals DCS and GCS to the data driving circuit DDC and the gate driving circuit GDC to control the data driving circuit DDC and the gate driving circuit GDC.

The data driving circuit DDC receives the image data DATA from the controller CTR and supplies data voltage to the plurality of data lines DL, thereby driving the plurality of data lines DL. Herein, the data driving circuit DDC is also referred to as a “source driving circuit'.”

The gate driving circuit GDC sequentially drives the plurality of gate lines GL by sequentially supplying scan signals to the plurality of gate lines GL. Herein, the gate driving circuit GDC is also referred to as a “scan driving circuit'.”

The gate driving circuit GDC sequentially supplies scan signals of “On-voltage” or “Off-voltage” to the plurality of gate lines GL under the control of the controller CTR.

When a specific gate line is opened by the gate driving circuit GDC, the data driving circuit DDC converts the image data DATA received from the controller CTR into an analog data voltage and supplies the analog data voltage to the plurality of data lines DL.

The data driving circuit DDC may be positioned on only one side (e.g., the top or bottom side) of the display panel PNL and, in some cases, the data driving circuit may be positioned on each of two opposite sides (e.g., both the top and bottom sides) of the display panel PNL depending on, e.g., driving schemes or panel designs.

The gate driving circuit GDC may be positioned on only one side (e.g., the left or right side) of the display panel PNL and, in some cases, the gate driving circuit GDR may be positioned on each of two opposite sides (e.g., both the left and right sides) of the display panel PNL depending on, e.g., driving schemes or panel designs.

The display device 100 according to embodiments of the disclosure may be an organic light emitting display device, a liquid crystal display device, a plasma display device, and the like.

When the display device 100 according to the embodiments of the disclosure is an organic light emitting display device, each subpixel SP arranged on the display panel PNL may be composed of a circuit element such as an organic light emitting diode (OLED) that is a self-luminous element, and a driving transistor for driving the OLED.

The type and number of circuit elements constituting each subpixel SP may be varied depending on functions to be provided and design schemes.

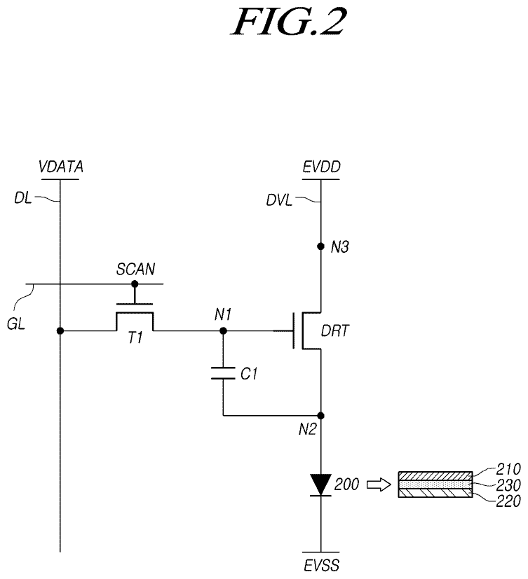

FIG. 2 illustrates a subpixel circuit of a display device according to embodiments of the disclosure.

Referring to FIG. 2, each subpixel SP may basically include an organic light emitting element 200 and a driving transistor DRT for driving the organic light emitting element 200.

Each subpixel SP may further include a first transistor T1 to transfer data voltage, VDATA, to a first node N1, which corresponds to a gate node of the driving transistor DRT, and a storage capacitor C1 to maintain the data voltage, VDATA, corresponding to an image signal voltage or a voltage corresponding to the data voltage VDATA for the time of one frame.

The organic light emitting element 200 may include a first electrode 210 (anode electrode or cathode electrode), an organic material layer 230, and a second electrode 220 (cathode electrode or anode electrode).

As an example, a base voltage EVSS may be applied to the second electrode 220 of the organic light emitting diode 200.

The driving transistor DRT supplies a driving current to the organic light emitting diode 200, thereby driving the organic light emitting diode 200.

The driving transistor DRT includes the first node N1, second node N2, and third node N3.

The first node N1 of the driving transistor DRT is a node corresponding to the gate node and may be electrically connected with the source node or drain node of the first transistor T1.

The second node N2 of the driving transistor DRT may be electrically connected with the first electrode 210 of the organic light emitting diode 200, and may be a source node or a drain node.

The third node N3 of the driving transistor DRT may be a node to which driving voltage EVDD is applied, may be electrically connected with a driving voltage line DVL for supplying the driving voltage EVDD, and may be the drain node or source node.

The first transistor T1 may be electrically connected between the data line DL and the first node N1 of the driving transistor DRT, and may be controlled by receiving the scan signal SCAN through the gate line and the gate node.

The storage capacitor C1 may be electrically connected between the first node N1 and second node N2 of the driving transistor DRT.

The storage capacitor C1 is an external capacitor intentionally designed to be outside the driving transistor DRT, but not a parasite capacitor (e.g., Cgs or Cgd) which is an internal capacitor present between the first node N1 and the second node N2 of the driving transistor DRT.

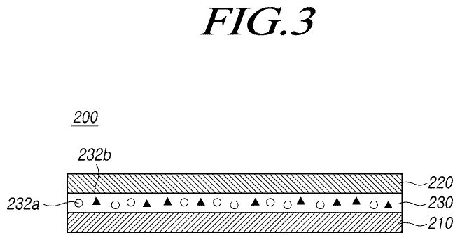

FIG. 3 is a cross-sectional view schematically illustrating an organic light emitting element according to embodiments of the disclosure.

The organic light emitting element 200 according to embodiments of the disclosure includes a first electrode 210, a second electrode 220, and an organic material layer 230 positioned between the first electrode 210 and the second electrode 220.

For example, the first electrode 210 may be the anode electrode, and the second electrode 220 may be the cathode electrode.

For example, the first electrode 210 may be a transparent electrode, and the second electrode 130 may be a reflective electrode. In another example, the first electrode 210 may be a reflective electrode, and the second electrode 130 may be a transparent electrode.

The organic material layer 230 is a layer positioned between the first electrode 210 and the second electrode 220 and including an organic material and may be composed of a plurality of layers.

The organic material layer 230 includes a first compound 232a and a second compound 232b. The first compound 232a and the second compound 232b are described below in detail. As the organic material layer 230 includes the above-described first compound 232a and second compound 232b, the organic light emitting element may have high efficiency, long lifespan, and/or low driving voltage.

The organic material layer 230 may include a light emitting layer. The organic layer 230 may further include at least one of a hole injection layer, a hole transport layer, an electron transport layer, and an electron injection layer.

For example, the organic material layer 230 may include a hole injection layer positioned on the first electrode 210, a hole transport layer positioned on the hole injection layer, a light emitting layer positioned on the hole transport layer, an electron transport layer positioned on the light emitting layer, and an electron injection layer positioned on the electron transport layer. In such an example, the first electrode 210 may be the anode electrode, and the second electrode 220 may be the cathode electrode.

The light emitting layer is a layer in which holes and electrons transferred from the first electrode 210 and the second electrode 130 meet to emit light and may include, e.g., a host material and a dopant.

In other words, the organic material layer 230 may include, e.g., a hole injection layer, a hole transport layer, a light emitting layer, an electron transport layer, and an electron injection layer. The hole injection layer may be positioned on the first electrode 210 as the anode electrode. The hole transport layer may be positioned on the hole injection layer. The light emitting layer may be positioned on the hole transport layer. The electron transport layer may be positioned on the light emitting layer. The electron injection layer may be positioned on the electron transport layer.

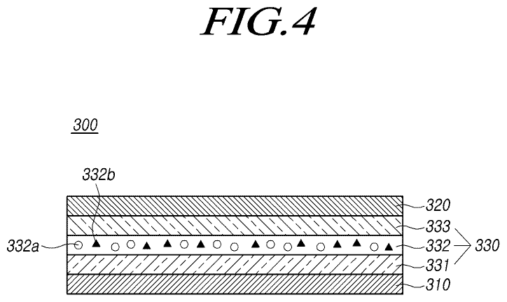

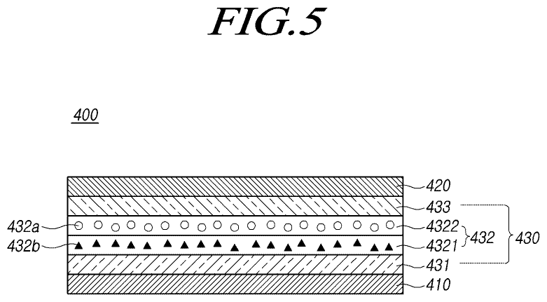

FIG. 4 is a cross-sectional view schematically illustrating an organic light emitting element 300 according to embodiments of the disclosure.

The organic light emitting element 300 may include a first electrode 310, a second electrode 320, and an organic material layer 330 positioned between the first electrode 310 and the second electrode 320.

The organic material layer 330 may include a first light emitting layer 331, a second light emitting layer 333, and a first layer 332 positioned between the first light emitting layer 331 and the second light emitting layer 333. In other words, the organic light emitting element 300 may be a tandem type organic light emitting element including two or more light emitting layers. The tandem type organic light emitting element may include a plurality of stacks each including a light emitting layer. For example, the tandem type organic light emitting element may include a first stack including a first light emitting layer 331 and a second stack including a second light emitting layer 333. In this example, the first stack may include additional functional layers in addition to the first light emitting layer 331. Further, the second stack may include additional functional layers in addition to the second light emitting layer 333.

The first light emitting layer 331 and the second light emitting layer 333 may be formed of the same material or different materials. The first light emitting layer 331 may emit light having a first color, and the second light emitting layer 333 may emit light having a second color. The first color and the second color may be the same as or different from each other.

The first layer 332 may include a first compound 332a and a second compound 332b. The first compound 332a and the second compound 332b are described below in detail. As the organic material layer 332 includes the first compound 332a and second compound 332b, the organic light emitting element may have high efficiency, long lifespan, and/or low driving voltage.

The first layer 332 may be a charge generation layer. For example, the organic light emitting element 300 may include a charge generation layer positioned between the first light emitting layer 331 and the second light emitting layer 333. The charge generation layer may include a p-type charge generation layer and an n-type charge generation layer.

The first stack may further include a functional layer in addition to the first light emitting layer 331. For example, the first stack may include a hole injection layer, a first hole transport layer, a first light emitting layer 331 and a first electron transport layer.

The second stack may further include a functional layer in addition to the second light emitting layer 333. For example, the second stack may include a second hole transport layer, a second light emitting layer 333, a second electron transport layer, and an electron injection layer.

The hole injection layer may be positioned on the first electrode 310 as the anode electrode. The first hole transport layer may be positioned on the hole injection layer. The first light emitting layer 331 may be positioned on the first hole transport layer. The first electron transport layer may be positioned on the first light emitting layer 331. The charge generation layer may be positioned on the first electron transport layer. The second hole transport layer may be positioned on the charge generation layer. The second light emitting layer 332 may be positioned on the second hole transport layer. The second electron transport layer may be positioned on the second light emitting layer 332. The electron injection layer may be positioned on the second electron transport layer. In this example, the first layer 333 may be a charge generation layer. In this example, the first compound 332a may be a p dopant of the charge generation layer, and the second compound 332b may be an n dopant of the charge generation layer.

The hole injection layer may include an amine-based compound. For example, the hole injection layer may include one or more of HATCN (1,4,5,8,9,11-hexaazatriphenylenehexacarbonitile) and NPD (N,N′-di(1-naphthyl)N,N′-diphenyl-(1,1-biphenyl)4,4′-diamine). However, the material for the hole injection layer is not limited to those described above, and may include other compounds that may be used as hole injection materials in the field of organic light emitting elements.

The first hole transport layer may include an amine-based compound. For example, the hole transport layer may include one or more of HATCN (1,4,5,8,9,11-hexaazatriphenylenehexacarbonitile) and NPD (N,N′-di(1-naphthyl)N,N′-diphenyl-(1,1-biphenyl)-4,4′-diamine). However, the material for the first hole transport layer is not limited to those described above, and may include other compounds that may be used as hole transport materials in the field of organic light emitting elements.

The first light emitting layer 331 may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound.

The first electron transport layer may include at least one of an azine-based compound and an imidazole-based compound. For example, the azine-based compound may be TmPyPB(1,3,5-tri(m-pyridin-3-ylphenyl)benzene). The imidazole-based compound may be TPBi (2,2′,2″-(1,3,5-benzinetriyl)-tris(1-phenyl-1H-benzimidazole). However, the material for the electron transport layer is not limited to those described above, and may include other compounds that may be used as electron transport materials in the field of organic light emitting elements.

The charge generation layer may include the second compound 332b as the n dopant. Further, the charge generation layer may include a phenanthroline-based compound as the n dopant. The phenanthroline-based compound may be bphen (bathophenanthroline). However, the n dopant that may be used in addition to the second compound 332b is not limited to those described above. When the first layer 332, which is the charge generation layer, includes the second compound 332b as the n dopant, the organic light emitting element 300 may have high efficiency, long lifespan, or low driving voltage.

The charge generation layer may include the first compound 332a as the p dopant. Further, the charge generation layer may further include an amine-based compound as the p dopant. The amine-based compound may be NPD (N,N′-di(1-naphthyl)N,N′-diphenyl-(1,1′-biphenyl)4,4′-diamine). However, the p dopant that may be used in addition to the first compound 332a is not limited to those described above. When the first layer 332, which is the charge generation layer, includes the first compound 332a as the p dopant, the organic light emitting element 300 may have high efficiency, long lifespan, or low driving voltage.

The second hole transport layer may include an amine-based compound. For example, the hole transport layer may include one or more of HATCN (1,4,5,8,9,11-hexaazatriphenylenehexacarbonitile) and NPD (N,N′-Di(1-naphthyl)N,N′-diphenyl-(1,1′-biphenyl)4,4′-diamine). However, the material for the second hole transport layer is not limited to those described above, and may include other compounds that may be used as hole transport materials in the field of organic light emitting elements.

The second light emitting layer 333 may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound. The carbazole-based compound may be CBP (4,4′-bis(N-carbazolyl)1,1′-biphenyl). The iridium-based compound may be Ir(ppy)₃(tris(2-phenylpyridine) iridium(III)). However, the material for the light emitting layer is not limited to those described above, and may include other compounds that may be used as light emitting layer materials in the field of organic light emitting elements.

The second electron transport layer may include at least one of an azine-based compound and an imidazole-based compound. For example, the azine-based compound may be TmPyPB (1,3,5-tri(m-pyridin-3-ylphenyl)benzene). The imidazole-based compound may be TPBi (2,2′,2″-(1,3,5-benzinetriyl)-tris(1-phenyl-1H-benzimidazole)). However, the material for the electron transport layer is not limited to those described above, and may include other compounds that may be used as electron transport materials in the field of organic light emitting elements.

The electron injection layer may include at least one of an azine-based compound and an imidazole-based compound. For example, the electron injection layer may include one or more of LiF and LiQ. However, the material for the electron injection layer is not limited to those described above, and may include other compounds that may be used as electron injection materials in the field of organic light emitting elements.

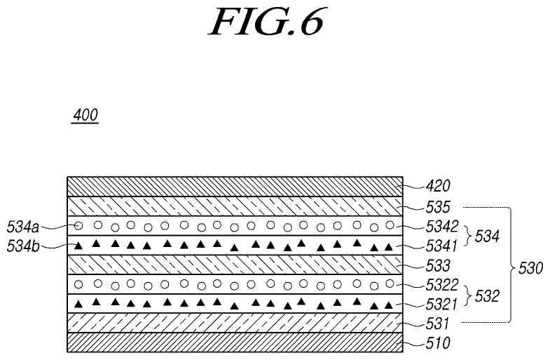

FIG. 5 is a cross-sectional view schematically illustrating an organic light emitting element 400 according to embodiments of the disclosure.

The organic light emitting element 400 may include a first electrode 410, a second electrode 420, and an organic material layer 430 positioned between the first electrode 410 and the second electrode 420.

The organic material layer 430 may include a first light emitting layer 431, a second light emitting layer 433, and a first layer 432 positioned between the first light emitting layer 431 and the second light emitting layer 433. In other words, the organic light emitting element 400 may be a tandem type organic light emitting element including two or more light emitting layers. The tandem type organic light emitting element may include a plurality of stacks each including a light emitting layer. For example, the tandem type organic light emitting element may include a first stack including a first light emitting layer 431 and a second stack including a second light emitting layer 433. In this example, the first stack may include additional functional layers in addition to the first light emitting layer 431. Further, the second stack may include additional functional layers in addition to the second light emitting layer 433.

The first light emitting layer 431 and the second light emitting layer 433 may be formed of the same material or different materials. The first light emitting layer 431 may emit light having a first color, and the second light emitting layer 433 may emit light having a second color. The first color and the second color may be the same as or different from each other.

The first layer 432 may include an n-type charge generation layer 4321 and a p-type charge generation layer 4322. In such an example, the first electrode 410 may be the anode electrode, and the second electrode 420 may be the cathode electrode.

The first layer 410 may include a first compound 432a and a second compound 432b. The first compound 432a and the second compound 432b are described below in detail. As the organic material layer 432 includes the first compound 432a and second compound 432b, the organic light emitting element may have high efficiency, long lifespan, or low driving voltage.

The first layer 432 may be a charge generation layer. For example, the organic light emitting element 400 may include a charge generation layer positioned between the first light emitting layer 431 and the second light emitting layer 433. The charge generation layer may include a p-type charge generation layer 4322 and an n-type charge generation layer 4321.

The first stack may further include a functional layer in addition to the first light emitting layer 431. For example, the first stack may include a hole injection layer, a first hole transport layer, a first light emitting layer 431 and a first electron transport layer.

The second stack may further include a functional layer in addition to the second light emitting layer 433. For example, the second stack may include a second hole transport layer, a second light emitting layer 433, a second electron transport layer, and an electron injection layer.

The hole injection layer may be positioned on the first electrode 410 as the anode electrode. The first hole transport layer may be positioned on the hole injection layer. The first light emitting layer 431 may be positioned on the first hole transport layer. The first electron transport layer may be positioned on the first light emitting layer 431. The n-type charge generation layer 4321 may be positioned on the first electron transport layer. The p-type charge generation layer may be positioned on the n-type charge generation layer 4321. The second hole transport layer may be positioned on the n-type charge generation layer 4321. The second light emitting layer 432 may be positioned on the second hole transport layer. The second electron transport layer may be positioned on the second light emitting layer 432. The electron injection layer may be positioned on the second electron transport layer. In this example, the first layer 433 may include an n-type charge generation layer 4321 and a p-type charge generation layer 4322. In this example, the first compound 432a may be a p dopant of the p-type charge generation layer 4322, and the second compound 432b may be an n dopant of the n-type charge generation layer 4321.

The hole injection layer may include an amine-based compound. For example, the hole injection layer may include one or more of HATCN (1,4,5,8,9,11-hexaazatriphenylenehexacarbonitile) and NPD (N,N′-di(1-naphthyl)N,N′-diphenyl-(1,1′-biphenyl)4,4′-diamine). However, the material for the hole injection layer is not limited to those described above, and may include other compounds that may be used as hole injection materials in the field of organic light emitting elements.

The first hole transport layer may include an amine-based compound. For example, the hole transport layer may include one or more of HATCN (1,4,5,8,9,11-hexaazatriphenylenehexacarbonitile) and NPD (N,N′-di(1-naphthyl)N,N′-diphenyl-(1,1′-biphenyl)4,4′-diamine). However, the material for the first hole transport layer is not limited to those described above, and may include other compounds that may be used as hole transport materials in the field of organic light emitting elements.

The first light emitting layer 431 may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound.

The first electron transport layer may include at least one of an azine-based compound and an imidazole-based compound. For example, the azine-based compound may be TmPyPB (1,3,5-tri(m-pyridin-3-ylphenyl)benzene). The imidazole-based compound may be TPBi (2,2′,2″-(1,3,5-benzinetriyl)-tris(1-phenyl-1H-benzimidazole). However, the material for the electron transport layer is not limited to those described above, and may include other compounds that may be used as electron transport materials in the field of organic light emitting elements.

The n-type charge generation layer 4321 may include the second compound 432b. Further, the n-type charge generation layer 4321 may include a phenanthroline-based compound as the n dopant. The phenanthroline-based compound may be bphen (bathophenanthroline). However, the n dopant that may be used in addition to the second compound 432b is not limited to those described above. When the n-type charge generation layer 4321 includes the second compound 432b, the organic light emitting element 400 may have high efficiency, long lifespan, or low driving voltage.

The p-type charge generation layer 4322 may include the first compound 432a as the p dopant. Further, the p-type charge generation layer 4322 may further include an amine-based compound as the p dopant. The amine-based compound may be NPD (N,N′-di(1-naphthyl)N,N′-diphenyl-(1,1′-biphenyl)4,4′-diamine). However, the p dopant that may be used in addition to the first compound 432a is not limited to those described above. When the p-type charge generation layer 4322 includes the first compound 432a, the organic light emitting element 400 may have high efficiency, long lifespan, and/or low driving voltage.

The second hole transport layer may include an amine-based compound. For example, the hole transport layer may include one or more of HATCN (1,4,5,8,9,11-hexaazatriphenylenehexacarbonitile) and NPD (N,N′-di(1-naphthyl)N,N′-diphenyl-(1,1′-biphenyl)4,4′-diamine). However, the material for the second hole transport layer is not limited to those described above, and may include other compounds that may be used as hole transport materials in the field of organic light emitting elements.

The second light emitting layer 433 may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound. The carbazole-based compound may be CBP (4,4′-bis(N-carbazolyl)1,1′-biphenyl). The iridium-based compound may be Ir(ppy)₃(tris(2-phenylpyridine) iridium(III)). However, the material for the light emitting layer is not limited to those described above, and may include other compounds that may be used as light emitting layer materials in the field of organic light emitting elements.

The second electron transport layer may include at least one of an azine-based compound and an imidazole-based compound. For example, the azine-based compound may be TmPyPB (1,3,5-tri(m-pyridin-3-ylphenyl)benzene). The imidazole-based compound may be TPBi (2,2′,2″-(1,3,5-benzinetriyl)-tris(1-phenyl-1H-benzimidazole). However, the material for the electron transport layer is not limited to those described above, and may include other compounds that may be used as electron transport materials in the field of organic light emitting elements.

The electron injection layer may include at least one of an azine-based compound and an imidazole-based compound. For example, the electron injection layer may include one or more of LiF and LiQ. However, the material for the electron injection layer is not limited to those described above, and may include other compounds that may be used as electron injection materials in the field of organic light emitting elements.

FIG. 6 is a cross-sectional view schematically illustrating an organic light emitting element 500 according to embodiments of the disclosure.

The organic light emitting element 500 may include a first electrode 510, a second electrode 520, and an organic material layer 530 positioned between the first electrode 510 and the second electrode 520.

The organic material layer 530 may include a first light emitting layer 531, a second light emitting layer 533, a third light emitting layer 535, a first layer 532 positioned between the first light emitting layer 531 and the second light emitting layer 533, and a charge generation layer 534 positioned between the second light emitting layer 533 and the third light emitting layer 535. The charge generation layer 534 may include a second n-type charge generation layer 5342 and a second p-type charge generation layer 5342 positioned on the second n-type charge generation layer 5342. In other words, the organic light emitting element 500 may be a tandem type organic light emitting element including three or more light emitting layers. The tandem type organic light emitting element may include a plurality of stacks each including a light emitting layer. For example, the tandem type organic light emitting element may include a first stack including a first light emitting layer 531, a second stack including a second light emitting layer 533, and a third stack including a third light emitting layer 535. In this example, the first stack may include additional functional layers in addition to the first light emitting layer 531. Further, the second stack may include additional functional layers in addition to the second light emitting layer 533. Further, the third stack may include additional functional layers in addition to the third light emitting layer 535.

The first light emitting layer 531, the second light emitting layer 533, and the third light emitting layer 535 may be formed of the same material or different materials. The first light emitting layer 531 may emit light having a first color, the second light emitting layer 533 may emit light having a second color, and the third light emitting layer 535 may emit light having a third color. The first color, the second color, and the third color may be the same as or different from each other.

The first layer 532 may include a first n-type charge generation layer 5321 and a first p-type charge generation layer 5322. In such an example, the first electrode 510 may be the anode electrode, and the second electrode 520 may be the cathode electrode.

The first layer 532 may include a first compound 532a and a second compound 532b. The first compound 532a and the second compound 532b are described below in detail. As the organic material layer 532 includes the first compound 532a and second compound 532b, the organic light emitting element may have high efficiency, long lifespan, and/or low driving voltage.

The first stack may further include a functional layer in addition to the first light emitting layer 531. For example, the first stack may include a hole injection layer, a first hole transport layer, a first light emitting layer 531 and a first electron transport layer.

The second stack may further include a functional layer in addition to the second light emitting layer 533. For example, the second stack may include a second hole transport layer, a second light emitting layer 533 and a second electron transport layer.

The third stack may further include a functional layer in addition to the third light emitting layer 535. For example, the third stack may include a third hole transport layer, a third light emitting layer 535, a third electron transport layer, and an electron injection layer.

The hole injection layer may be positioned on the first electrode 510 as the anode electrode. The first hole transport layer may be positioned on the hole injection layer. The first light emitting layer 531 may be positioned on the first hole transport layer. The first electron transport layer may be positioned on the first light emitting layer 531. The charge generation layer 534 may include an n-type charge generation layer and a p-type charge generation layer. The first n-type charge generation layer 5321 may be positioned on the first electron transport layer. The first p-type charge generation layer 5322 may be positioned on the first n-type charge generation layer 5321. The second hole transport layer may be positioned on the first n-type charge generation layer 5321. The second light emitting layer 532 may be positioned on the second hole transport layer. The second electron transport layer may be positioned on the second light emitting layer 532. The second n-type charge generation layer 5341 may be positioned on the second electron transport layer. The second p-type charge generation layer 5342 may be positioned on the second n-type charge generation layer 5341. The third hole transport layer may be positioned on the second p-type charge generation layer 5342. The third light emitting layer 535 may be positioned on the third hole transport layer. The third electron transport layer may be positioned on the third light emitting layer 535. The electron injection layer may be positioned on the third electron transport layer. In this example, the first layer 532 may include a first n-type charge generation layer 5321 and a first p-type charge generation layer 5322. In this example, the first compound 532a may be a p dopant of the first p-type charge generation layer 5322, and the second compound 532b may be an n dopant of the first n-type charge generation layer 5321. The second n-type charge generation layer 5341 may include an n dopant 534b. The n dopant 534b may be the same as or different from the second compound 532b. The second p-type charge generation layer 5342 may include a p dopant 534a. The p dopant 534a may be the same as or different from the first compound 532a.

The hole injection layer may include an amine-based compound. For example, the hole injection layer may include one or more of HATCN (1,4,5,8,9,11-hexaazatriphenylenehexacarbonitile) and NPD (N,N′-Di(1-naphthyl)-N,N′-diphenyl-(1,1′-biphenyl)4,4′-diamine). However, the material for the hole injection layer is not limited to those described above, and may include other compounds that may be used as hole injection materials in the field of organic light emitting elements.

The first hole transport layer may include an amine-based compound. For example, the hole transport layer may include one or more of HATCN (1,4,5,8,9,11-hexaazatriphenylenehexacarbonitile) and NPD (N,N′-Di(1-naphthyl)-N,N′-diphenyl-(1,1′-biphenyl)4,4′-diamine). However, the material for the first hole transport layer is not limited to those described above, and may include other compounds that may be used as hole transport materials in the field of organic light emitting elements.

The first light emitting layer 531 may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound.

The first electron transport layer may include at least one of an azine-based compound and an imidazole-based compound. For example, the azine-based compound may be TmPyPB(1,3,5-tri(m-pyridin-3-ylphenyl)benzene). The imidazole-based compound may be TPBi (2,2′,2″-(1,3,5-benzinetriyl)-tris(1-phenyl-1H-benzimidazole)). However, the material for the electron transport layer is not limited to those described above, and may include other compounds that may be used as electron transport materials in the field of organic light emitting elements.

The first n-type charge generation layer 5321 may include the second compound 532b. Further, the first n-type charge generation layer 5321 may include a phenanthroline-based compound as the n dopant. The phenanthroline-based compound may be bphen (bathophenanthroline). However, the n dopant that may be used in addition to the second compound 532b is not limited to those described above. When the first n-type charge generation layer 5321 includes the second compound 532b, the organic light emitting element 500 may have high efficiency, long lifespan, or low driving voltage.

The first p-type charge generation layer 5322 may include the first compound 532a as the p dopant. Further, the first p-type charge generation layer 5322 may further include an amine-based compound as the p dopant. The amine-based compound may be NPD (N,N′-di(1-naphthyl)N,N′-diphenyl-(1,1′-biphenyl)4,4′-diamine). However, the p dopant that may be used in addition to the first compound 532a is not limited to those described above. When the first p-type charge generation layer 5322 includes the first compound 532a, the organic light emitting element 500 may have high efficiency, long lifespan, or low driving voltage.

The second hole transport layer may include an amine-based compound. For example, the hole transport layer may include one or more of HATCN (1,4,5,8,9,11-hexaazatriphenylenehexacarbonitile) and NPD (N,N′-Di(1-naphthyl)N,N′-diphenyl-(1,1′-biphenyl)4,4′-diamine). However, the material for the second hole transport layer is not limited to those described above, and may include other compounds that may be used as hole transport materials in the field of organic light emitting elements.

The second light emitting layer 533 may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound. The carbazole-based compound may be CBP (4,4′-bis(N-carbazolyl)1,1′-biphenyl). The iridium-based compound may be Ir(ppy)₃(tris(2-phenylpyridine) Iridium(III). However, the material for the light emitting layer is not limited to those described above, and may include other compounds that may be used as light emitting layer materials in the field of organic light emitting elements.

The second electron transport layer may include at least one of an azine-based compound and an imidazole-based compound. For example, the azine-based compound may be TmPyPB(1,3,5-tri(m-pyridin-3-ylphenyl)benzene). The imidazole-based compound may be TPBi (2,2′,2″-(1,3,5-benzinetriyl)-tris(1-phenyl-1H-benzimidazole). However, the material for the electron transport layer is not limited to those described above, and may include other compounds that may be used as electron transport materials in the field of organic light emitting elements.

The third hole transport layer may include an amine-based compound. For example, the hole transport layer may include one or more of HATCN (1,4,5,8,9,11-hexaazatriphenylenehexacarbonitile) and NPD (N,N′-Di(1-naphthyl)N,N′-diphenyl-(1,1′-biphenyl)4,4′-diamine). However, the material for the third hole transport layer is not limited to those described above, and may include other compounds that may be used as hole transport materials in the field of organic light emitting elements.

The third light emitting layer 535 may be a fluorescent light emitting layer or a phosphorescent light emitting layer. The fluorescent light emitting layer may include one or more of a boron-based compound, an anthracene-based compound, and a pyrene-based compound. The phosphorescent light emitting layer may include at least one of a carbazole-based compound and an iridium-based compound. The carbazole-based compound may be CBP (4,4′-bis(N-carbazolyl)-1,1′-biphenyl). The iridium-based compound may be Ir(ppy)₃(tris(2-phenylpyridine) Iridium(III). However, the material for the light emitting layer is not limited to those described above, and may include other compounds that may be used as light emitting layer materials in the field of organic light emitting elements.

The third electron transport layer may include at least one of an azine-based compound and an imidazole-based compound. For example, the azine-based compound may be TmPyPB(1,3,5-tri(m-pyridin-3-ylphenyl)benzene). The imidazole-based compound may be TPBi (2,2′,2″-(1,3,5-benzinetriyl)-tris(1-phenyl-1H-benzimidazole). However, the material for the electron transport layer is not limited to those described above, and may include other compounds that may be used as electron transport materials in the field of organic light emitting elements.

The electron injection layer may include at least one of an azine-based compound and an imidazole-based compound. For example, the electron injection layer may include one or more of LiF and LiQ. However, the material for the electron injection layer is not limited to those described above, and may include other compounds that may be used as electron injection materials in the field of organic light emitting elements.

The above-described first compounds 232a, 332a, 432a, and 532a are described below.

The first compounds 232a, 332a, 432a, and 532a are represented by chemical formula 1 below.

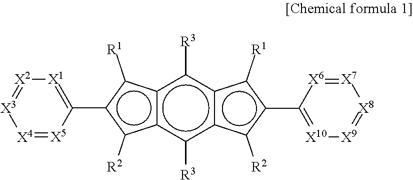

Hereinafter, chemical formula 1 is described.

R¹ and R² are each independently selected from the group consisting of hydrogen, deuterium, tritium, halogen, a cyano group, an alkyl group of C₁-C₅₀, a haloalkyl group of C₁-C₅₀, an alkoxy group of C₁-C₃₀, a haloalkoxy group of C₁-C₃₀, an aryl group of C₆-C₆₀, a haloaryl group of C₆-C₆₀, a heterocyclic group of C₂-C₆₀ including at least one heteroatom among O, N, S, Si and P, a haloheterocyclic group of C₂-C₆₀ including at least one heteroatom among O, N, S, Si and P, and malononitrile.

For example, R¹ to R³ may be each independently selected from the group consisting of hydrogen, deuterium, tritium, a cyano group and malononitrile.

R³ is each independently selected from the group consisting of hydrogen, deuterium, tritium, halogen, a cyano group, a malononitrile group, an alkyl group of C₁-C₅₀, a haloalkyl group of C₁-C₅₀, an alkoxy group of C₁-C₃₀, a haloalkoxy group of C₁-C₃₀, an aryl group of C₆-C₆₀, a haloaryl group of C₆-C₆₀, a heterocyclic group of C₂-C₆₀ including at least one heteroatom among O, N, S, Si and P, and a haloheterocyclic group of C₂-C₆₀ including at least one heteroatom among O, N, S, Si and P.

For example, R³ may be each independently selected from the group consisting of hydrogen, deuterium, tritium, halogen and a cyano group.

X¹ to X⁵ are each independently CR^{a} or N, and at least two of X¹ to X⁵ are CR^{a}.
R^{a} may be each independently selected from among hydrogen, deuterium, tritium, halogen, a cyano group, an alkyl group of C₁-C₅₀, and an alkoxy group of C₁-C_{50.} Further, at least one R^{a} is a halogen or cyano group. In other words, at least one of R^{a} is an electron withdrawing group (EWG).

For example, R³ may be each independently selected from the group consisting of hydrogen, deuterium, tritium, halogen and a cyano group. Further, at least one R^{a} may be a halogen or cyano group, preferably at least one Ra is a halogen or cyano group.

X⁶ to X¹⁰ are each independently CR^{b} or N, and at least two of X⁶ to X¹⁰ are CR^{b}.

R^{b} is each independently selected from among hydrogen, deuterium, tritium, halogen, a cyano group, an alkyl group of C₁-C₅₀, and an alkoxy group of C₁-C₅₀. Further, at least one R^{b} is a halogen or cyano group. In other words, at least one of Rb is an electron withdrawing group (EWG).

For example, R^{b} may be each independently selected from the group consisting of hydrogen, deuterium, tritium, halogen and a cyano group. Further, at least one R^{b} may be a halogen or cyano group, preferably at least one Rb is a halogen or cyano group.

Independently each of R¹ to R³, R^{a} and R^{b} in chemical formula 1 may be further substituted. E.g., in case each of R1 to R3, Ra and Rb in chemical formula 1 is independently selected from an alkyl group, a haloalkyl group, an alkoxy group, a haloalkoxy group, an aryl group, a haloaryl group, a heterocyclic group, and a haloheterocyclic group, this group may be further substituted with one or more substituents selected from the group consisting of deuterium, a nitro group, a cyano group, an amino group, an alkoxy group of C₁-C₂₀, a haloalkoxy group of C₁-C₂₀, an alkyl group of C₁-C₂₀, a haloalkyl group of C₁-C₂₀, an alkenyl group of C₂-C₂₀, an alkynyl group of C₂-C₂₀, an aryl group of C₆-C₂₀, an aryl group of C₆-C₂₀ substituted with deuterium, a fluorenyl group, a heterocyclic group of C₂-C₂₀, an alkylsilyl group of C₃-C_{60,} an arylsilyl group of C₁₈-C₆₀, and an alkylarylsilyl group of C₈-C₆₀.

The above-described first compounds 232a, 332a, 432a, and 532a may be represented by either chemical formula 3 or chemical formula 4 below.

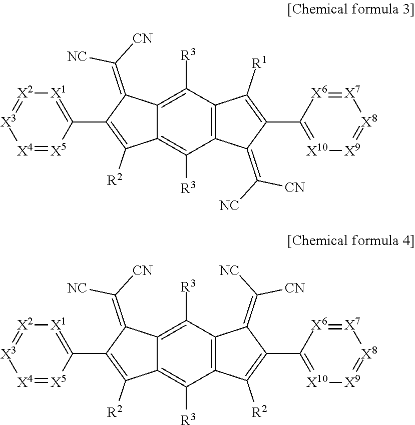

In chemical formula 3 and chemical formula 4, R¹ to R³ and X¹ to X¹⁰ may be the same as R¹ to R³ and X¹ to X¹⁰ defined in chemical formula 1 described above.

The above-described first compounds 232a, 332a, 432a, and 532a may be represented by either chemical formula 5 or chemical formula 6 below.

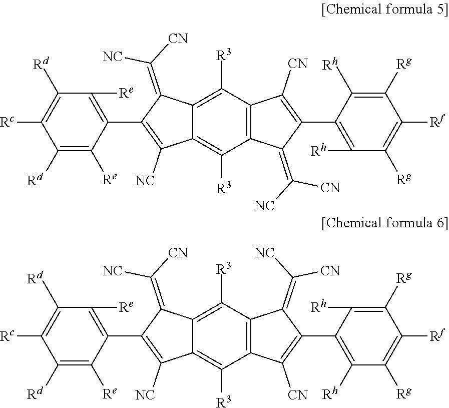

Hereinafter, chemical formula 5 and chemical formula 6 are described.

R^{c} and R^{d} may be each independently selected from among hydrogen, deuterium, tritium, halogen, a cyano group, an alkyl group of C₁-C₅₀, and an alkoxy group of C₁-C₅₀.

When R^{c} and R^{d} are a haloalkoxy group, Rc and R^{d} may be a haloalkoxy group of C₁-C₃₀, a haloalkoxy group of C₁-C₁₅, or a haloalkoxy group of C₁-C₁₀.

Each R^{e} is independently hydrogen, deuterium, tritium, halogen, or a cyano group.

R^{f} and R^{g} may be each independently selected from among hydrogen, deuterium, tritium, halogen, a cyano group, an alkyl group of C₁-C₅₀, and an alkoxy group of C₁-C₅₀.

Each R^{h} is independently hydrogen, deuterium, tritium, halogen, or a cyano group.

R³ is the same as R³ defined in chemical formula 1.

Hereinafter, chemical formula 5 is described in more detail.

R^{c} may be a halogen or cyano group. In other words, R^{c} may be an electron withdrawing group (EWG).

One R^{d} may be hydrogen, deuterium, or tritium, and the other R^{d} may be a halogen or cyano group. In this example, one R^{d} of the two R^{d}s may be an electron withdrawing group (EWG). In another example two R^{d}s may be a halogen or cyano group. In this example, both the R^{d}s may be an electron withdrawing group (EWG).

One R^{e} may be hydrogen, deuterium, or tritium, and the other R^{e} may be a halogen or cyano group. For example, one R^{e} may be hydrogen, and the other R^{e} may be a halogen or cyano group. In the six-membered ring connected to the indacene moiety in the center, the halogen or cyano group which is an electron withdrawing group (EWG) may be substituted at only one of the two carbons which are in the ortho positions of the carbons connected to the indacene moiety in form of the six-membered ring connected to the indacene moiety. By including the compounds 232a, 332a, 432a, and 532a having such a structure, the organic light emitting elements 200, 300, 400, and 500 may have excellent efficiency, long lifespan and/or low driving voltage.

In the benzene ring in which R^{c} and R^{d} are substituted in chemical formula 5, R^{c}, which is in a para position in relation to the indacene moiety, may be an electron withdrawing group (EWG), and at least one of R^{c}, which is in a para position in relation to the indacene moiety, and R^{d}, which is in a meta position in relation to the indacene moiety, is an electron withdrawing group (EWG). For example, at least one of Rc and Rd may be an electron withdrawing group (EWG) other than a cyano group. The organic light emitting elements 200, 300, 400, and 500 including the compounds 232a, 332a, 432a, and 532a having such a structure and represented in chemical formula 5 have excellent efficiency, a long lifespan, or a low driving voltage.

R^{f} may be a halogen or cyano group. In other words, R^{f} may be an electron withdrawing group (EWG).

One R^{g} may be hydrogen, deuterium, or tritium, and the other R^{g} may be a halogen or a cyano group. In this example, one R^{g} of the two R^{g}s may be an electron withdrawing group (EWG). In another example two R^{g}s may be a halogen or cyano group. In this example, both the Ros may be an electron withdrawing group (EWG).

One R^{h} may be hydrogen, deuterium, or tritium, and the other R^{h} may be a halogen or cyano group. For example, one R^{h} may be hydrogen, and the other R^{h} may be a halogen or cyano group. In the six-membered ring connected to the indacene moiety in the center, the halogen or cyano group which is an electron withdrawing group (EWG) may be substituted at only one of the two carbons which are in the ortho positions of the carbons connected to the indacene derivative in form of the six-membered ring connected to the indacene moiety. By including the compounds 232a, 332a, 432a, and 532a having such a structure, the organic light emitting elements 200, 300, 400, and 500 may have excellent efficiency, long lifespan and/or low driving voltage.

In the benzene ring in which R^{f} and R^{g} are substituted in chemical formula 5, R^{f}, which is in a para position in relation to the indacene moiety, may be an electron withdrawing group (EWG), and at least one of R^{f} which is in a para position in relation to the indacene moiety and R^{g}, which is in a meta position in relation to the indacene moiety, is an electron withdrawing group (EWG). For example, at least one of R^{f} and R^{g} may be an electron withdrawing group (EWG) other than a cyano group. The organic light emitting elements 200, 300, 400, and 500 including the compounds 232a, 332a, 432a, and 532a having such a structure and represented in chemical formula 5 have excellent efficiency, a long lifespan, or a low driving voltage.

Hereinafter, chemical formula 6 is described in more detail.

R^{c} may be a halogen or cyano group. In other words, R^{c} may be an electron withdrawing group (EWG).

One R^{d} may be hydrogen, deuterium, or tritium, and the other R^{d} may be a halogen or a cyano group. In this example, one R^{d} of the two R^{d}s may be an electron withdrawing group (EWG). In another example two R^{d}s may be a halogen or cyano group. In this example, both the R^{d}s may be an electron withdrawing group (EWG).

In the benzene ring in which R^{c} and R^{d} are substituted in chemical formula 6, R^{c}, which is in a para position in relation to the indacene moiety, may be an electron withdrawing group (EWG), and at least one of R^{c}, which is in a para position in relation to the indacene moiety, and R^{d}, which is in a meta position in relation to the indacene moiety, is an electron withdrawing group (EWG). For example, at least one of R^{c} and R^{d} may be an electron withdrawing group (EWG) other than a cyano group. The organic light emitting elements 200, 300, 400, and 500 including the compounds 232a, 332a, 432a, and 532a having such a structure and represented in chemical formula 6 have excellent efficiency, a long lifespan, and/or a low driving voltage.

One R^{e} may be hydrogen, deuterium, or tritium, and the other R^{e} may be a halogen or cyano group. For example, one R^{e} may be hydrogen, and the other R^{e} may be a halogen or cyano group. In the six-membered ring connected to the indacene moiety in the center, the halogen or cyano group which is an electron withdrawing group (EWG) may be substituted at only one of the two carbons which are in the ortho positions of the carbons connected to the indacene derivative in form of the six-membered ring connected to the indacene moiety. By including the compounds 232a, 332a, 432a, and 532a having such a structure, the organic light emitting elements 200, 300, 400, and 500 may have excellent efficiency, long lifespan and/or low driving voltage.

R^{f} may be a halogen or cyano group. In other words, Rfmay be an electron withdrawing group (EWG).

One R^{g} may be hydrogen, deuterium, or tritium, and the other R^{g} may be a halogen or cyano group. In this example, one R^{g} of the two R^{g}s may be an electron withdrawing group (EWG). In another example two R^{g}s may be a halogen or cyano group. In this example, both the R^{g}s may be an electron withdrawing group (EWG).

One R^{h} may be hydrogen, deuterium, or tritium, and the other R^{h} may be a halogen or cyano group. For example, one R^{h} may be hydrogen, and the other R^{h} may be a halogen or cyano group. In the six-membered ring connected to the indacene moiety in the center, the halogen or cyano group which is an electron withdrawing group (EWG) may be substituted at only one of the two carbons which are in the ortho positions of the carbons connected to the indacene derivative in form of the six-membered ring connected to the indacene moiety. By including the compounds 232a, 332a, 432a, and 532a having such a structure, the organic light emitting elements 200, 300, 400, and 500 may have excellent efficiency, long lifespan and/or low driving voltage.

In R^{c} to R^{h} in chemical formulas 5 and 6, the haloalkyl group and haloalkoxy group may be further substituted with one or more substituents selected from the group consisting of deuterium, a nitro group, a cyano group, an amino group, an alkoxy group of C₁-C₂₀, a haloalkoxy group of C₁-C₂₀, an alkyl group of C₁-C₂₀, a haloalkyl group of C₁-C₂₀, an alkenyl group of C₂-C₂₀, an alkynyl group of C₂-C₂₀, an aryl group of C₆-C₂₀, an aryl group of C₆-C₂₀ substituted with deuterium, a fluorenyl group, a heterocyclic group of C₂-C₂₀, an alkylsilyl group of C₃-C₆₀, an arylsilyl group of C₁₈-C₆₀, and an alkylarylsilyl group of C₈-C₆₀.

The above-described first compounds 232a, 332a, 432a, and 532a may be represented by any one of chemical formula 7 to chemical formula 16 below. More specifically, the compound represented by chemical formula 3 described above may be represented by any one of chemical formulas 7 to 11, and the compound represented by chemical formula 4 described above may be represented by any one of chemical formulas 12 to 16.

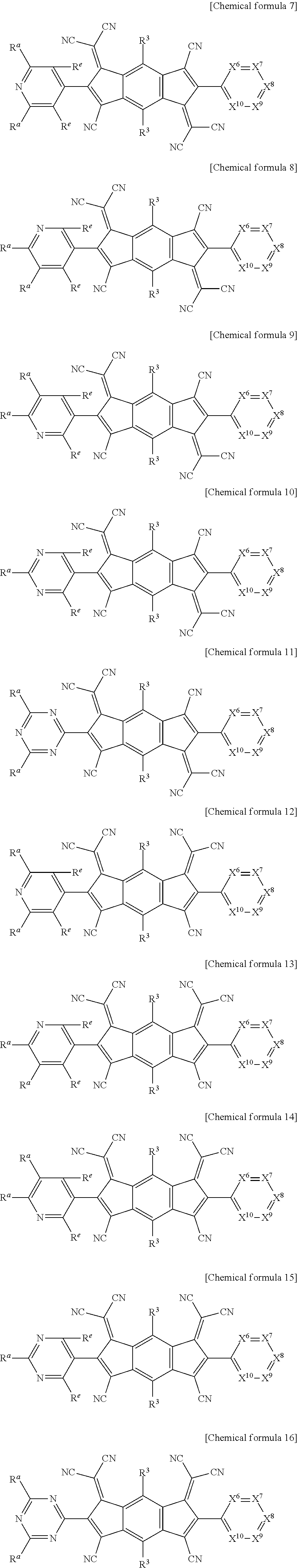

Hereinafter, chemical formulas 7 to chemical formula 16 are described.

R^{a}, R³ and X⁶ to X¹⁰ are the same as R^{a}, R³ and X⁴ to X¹⁰ defined in chemical formula 1 described above.

R^{e} may be each independently selected from the group consisting of hydrogen, deuterium, tritium, halogen and a cyano group. For example, one R^{e} may be hydrogen, and the other R^{c} may be a halogen or cyano group. In the six-membered ring connected to the indacene moiety in the center, the halogen or cyano group which is an electron withdrawing group (EWG) may be substituted at only one of the two carbons which are in the ortho positions of the carbons connected to the indacene moiety in form of the six-membered ring connected to the indacene moiety. By including the compounds 232a, 332a, 432a, and 532a having such a structure, the organic light emitting elements 200, 300, 400, and 500 may have excellent efficiency, long lifespan and/or low driving voltage.

Hereinafter, chemical formula 7 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

Hereinafter, chemical formula 8 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

Hereinafter, chemical formula 9 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

Hereinafter, chemical formula 10 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

Hereinafter, chemical formula 12 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

Hereinafter, chemical formula 13 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

Hereinafter, chemical formula 14 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

Hereinafter, chemical formula 15 is described in more detail.

One R^{e} may be hydrogen, deuterium, or tritium, and the other R^{e} may be a halogen or cyano group. For example, one R^{e} may be hydrogen, and the other R^{e} may be a halogen or cyano group.

In chemical formula 7 to chemical formula 16 described above, at least one electron withdrawing group (EWG) may be substituted at the heterocyclic group bonded to the indacene moiety. The organic light emitting elements 200, 300, 400, and 500 including the compounds 232a, 332a, 432a, and 532a having such a structure have excellent efficiency, a long lifespan, and/or a low driving voltage.

Further, an electron withdrawing group (EWG) is substituted at only one of the two carbons that are in the ortho positions of the carbon atoms of the 6-membered ring bonded to the indacene moiety. The organic light emitting elements 200, 300, 400, and 500 including the compounds 232a, 332a, 432a, and 532a having such a structure have excellent efficiency, a long lifespan, and/or a low driving voltage.

The first compounds 232a, 332a, 432a, and 532a may be one or more of the following compounds.

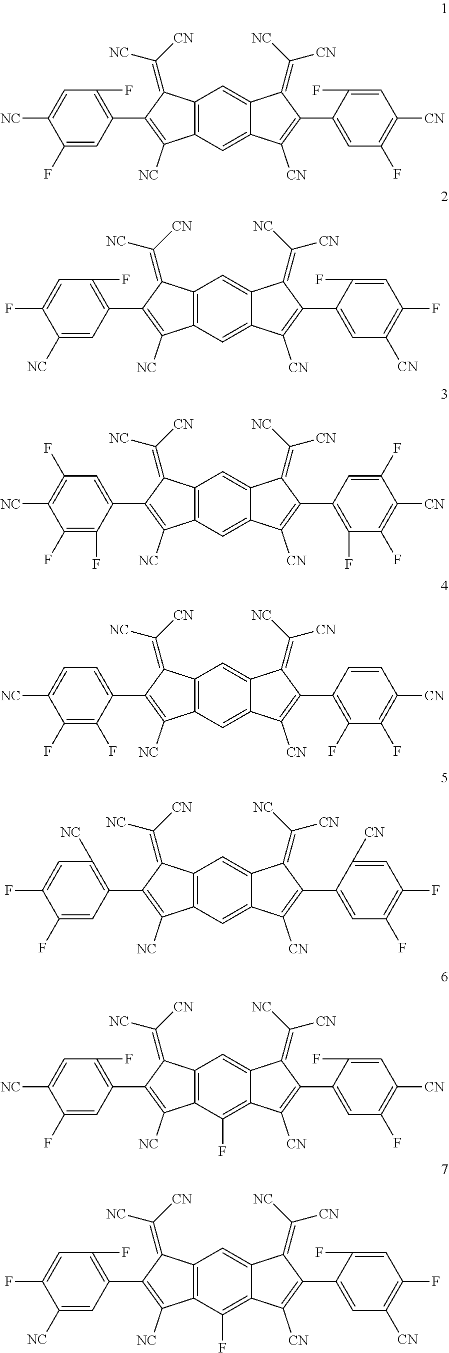

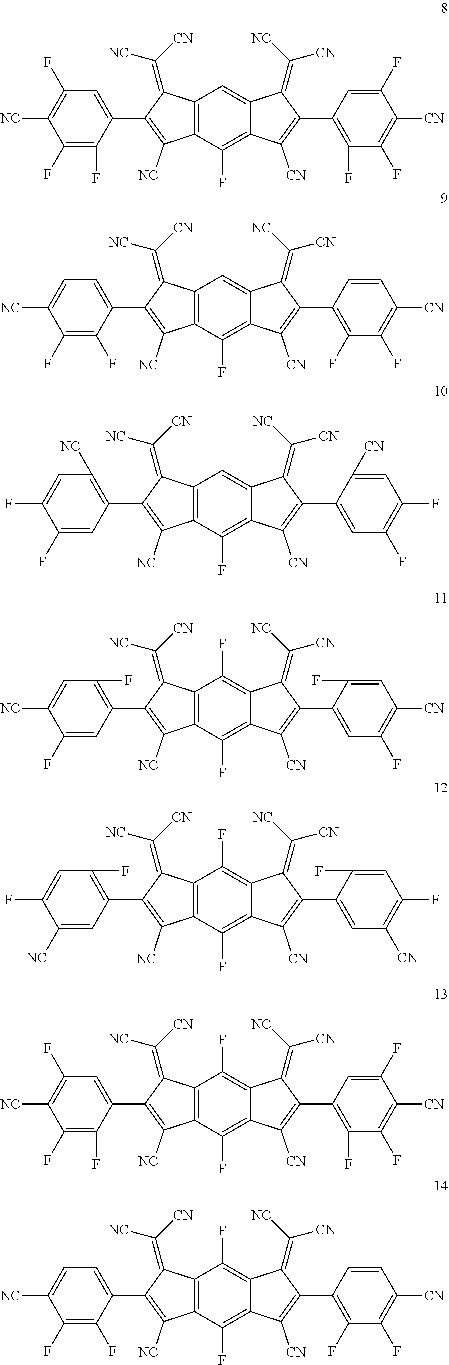

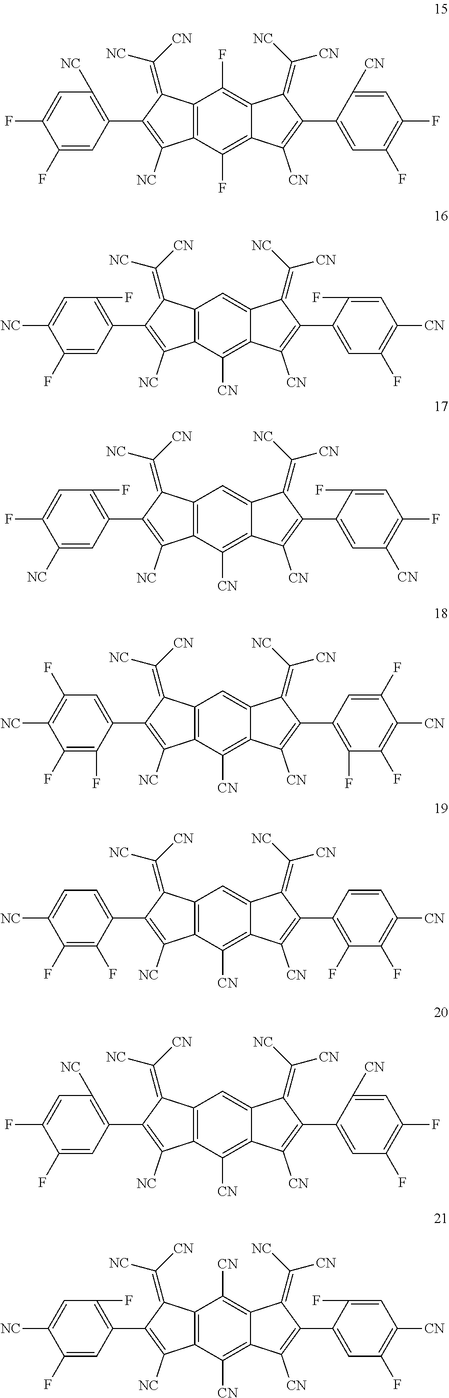

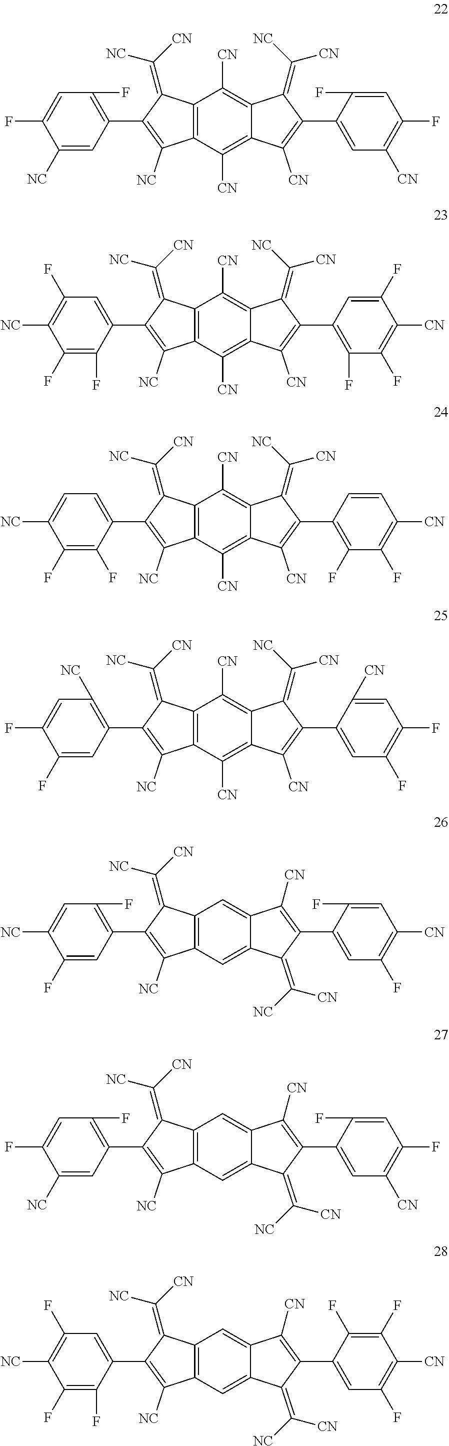

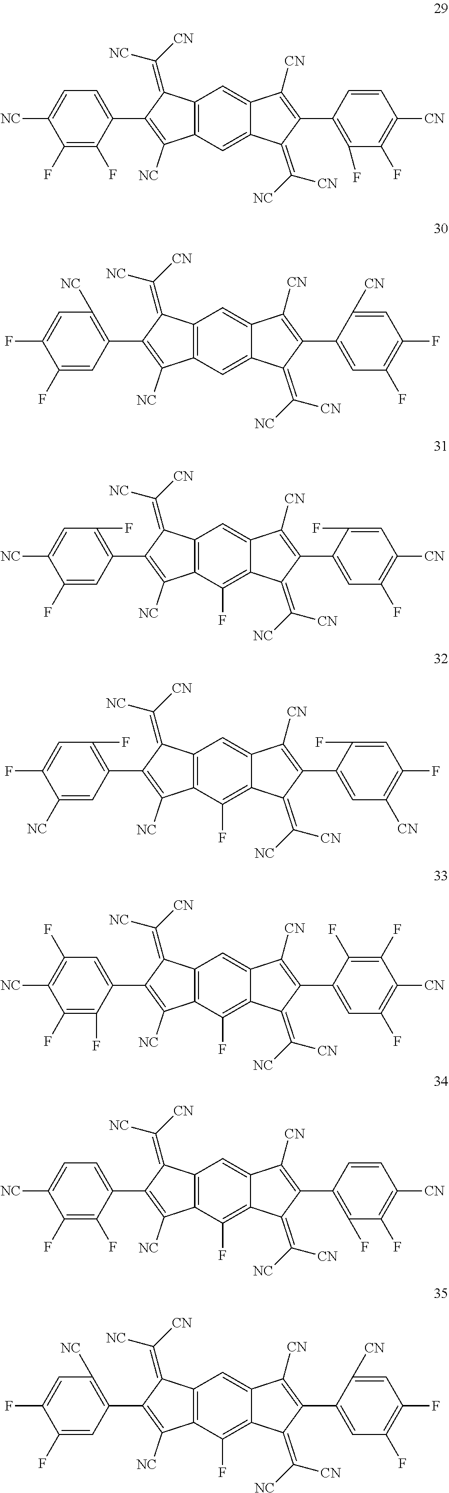

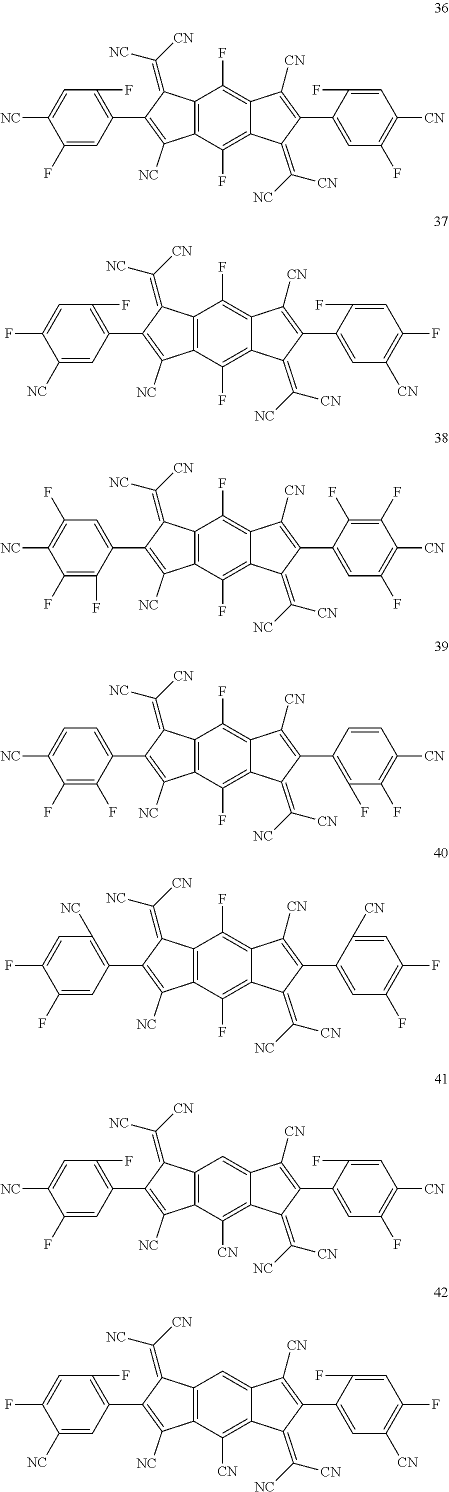

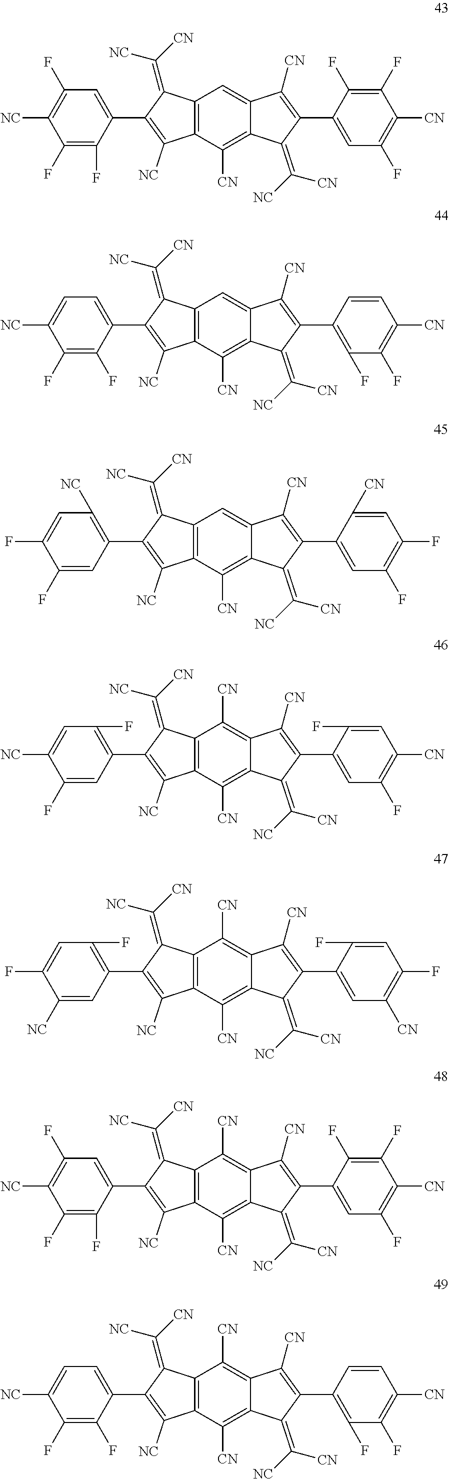

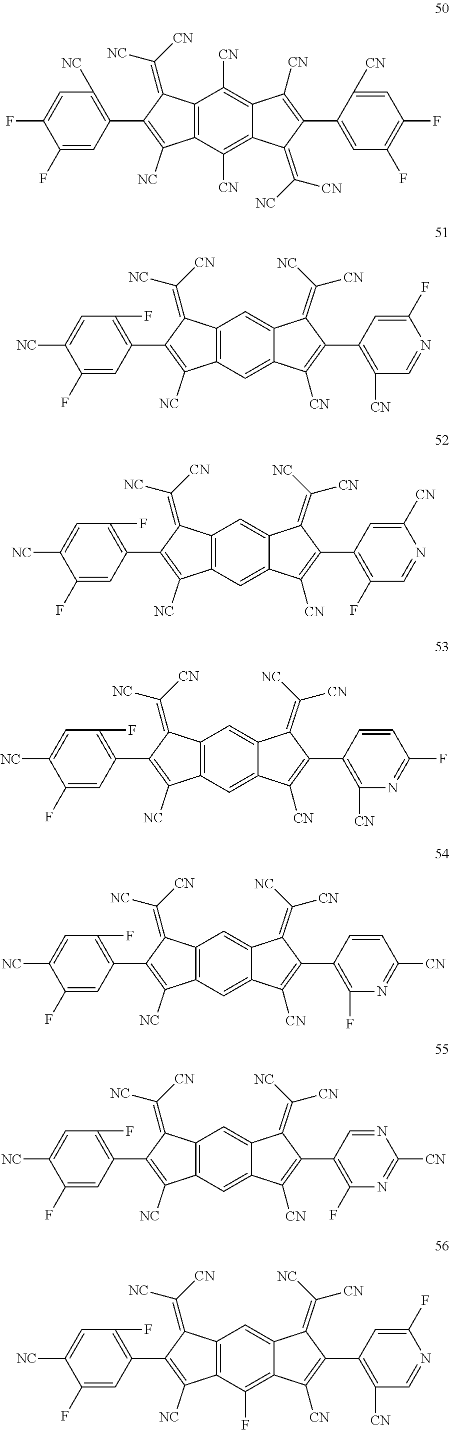

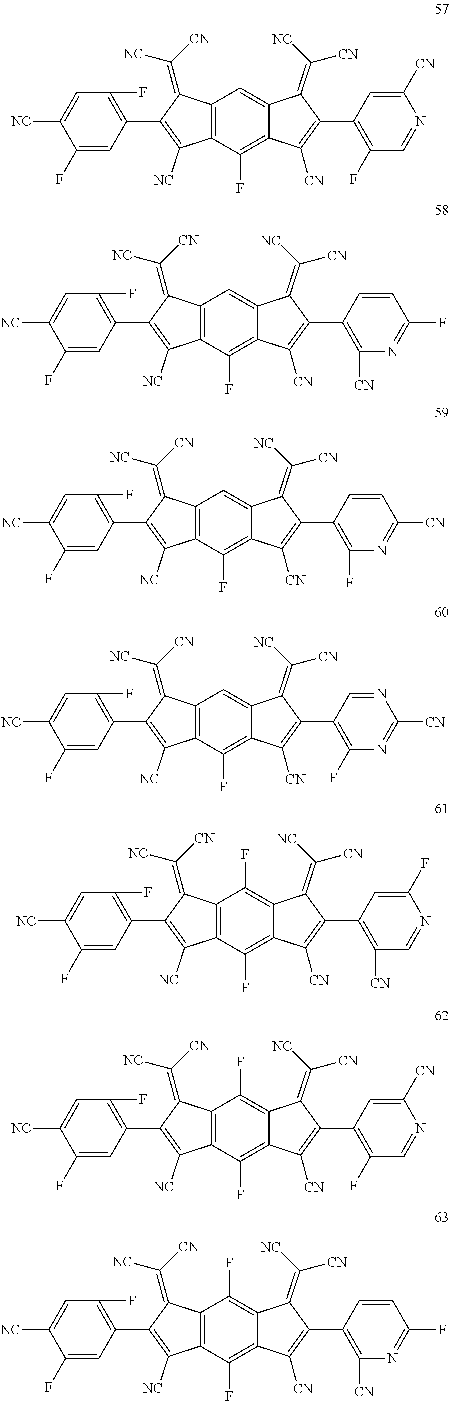

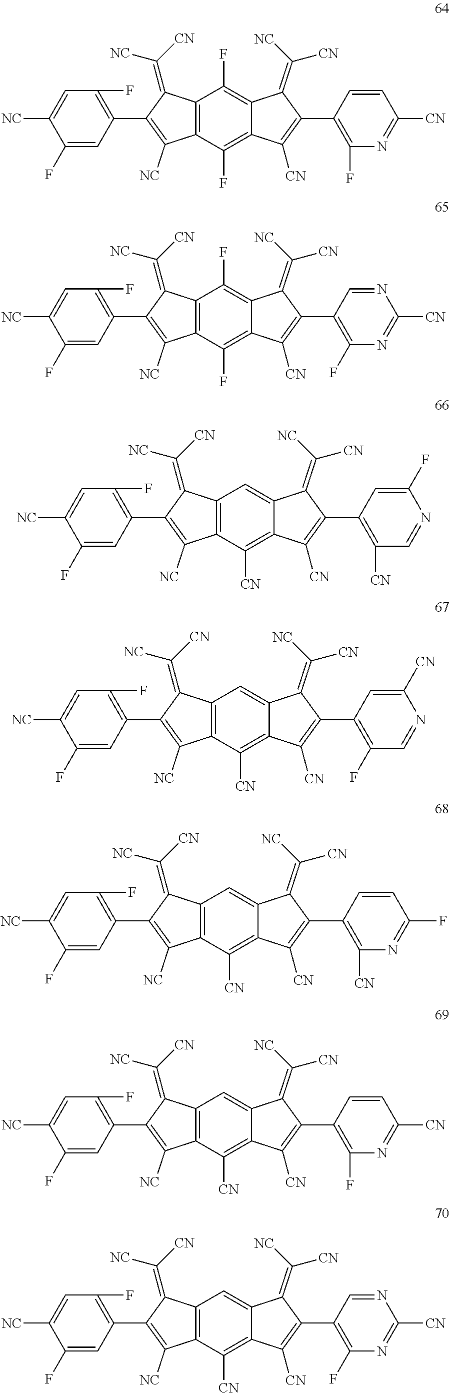

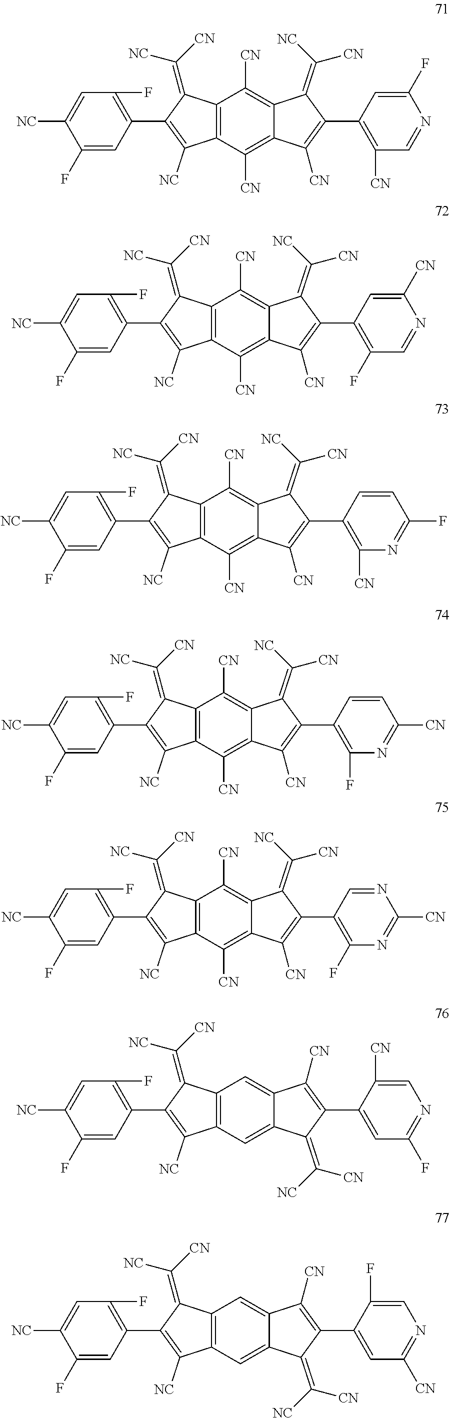

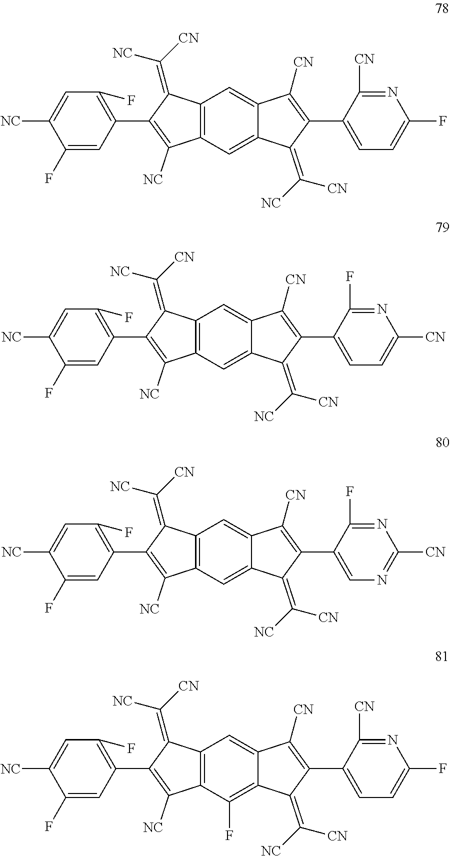 [SMILES: N#CC(C#N)=C1C(c2cc(F)c(C#N)cc2F)=C(C#N)c2cc3c(cc21)C(C#N)=C(c1ccc(F)nc1C#N)C3=C(C#N)C#N ; N#CC(C#N)=C1C(c2cc(F)c(C#N)cc2F)=C(C#N)c2cc3c(cc21)C(C#N)=C(c1ccc(C#N)nc1F)C3=C(C#N)C#N ; N#CC(C#N)=C1C(c2cc(F)c(C#N)cc2F)=C(C#N)c2cc3c(cc21)C(C#N)=C(c1cnc(C#N)nc1F)C3=C(C#N)C#N ; N#CC(C#N)=C1C(c2cc(F)c(C#N)cc2F)=C(C#N)c2c1cc1c(c2F)C(=C(C#N)C#N)C(c2ccc(F)nc2C#N)=C1C#N]

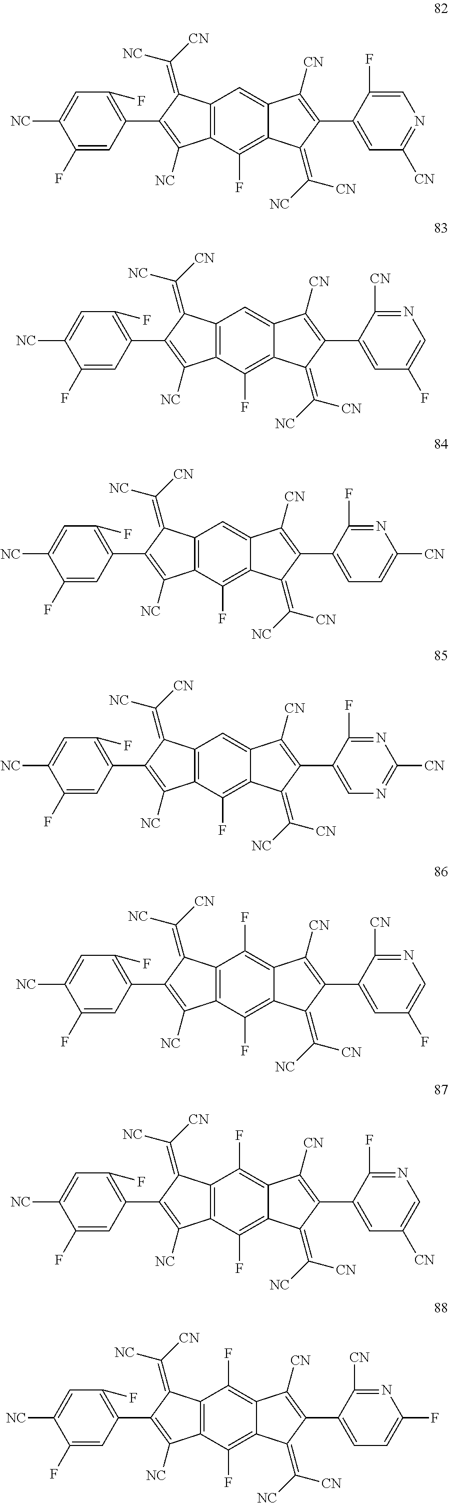

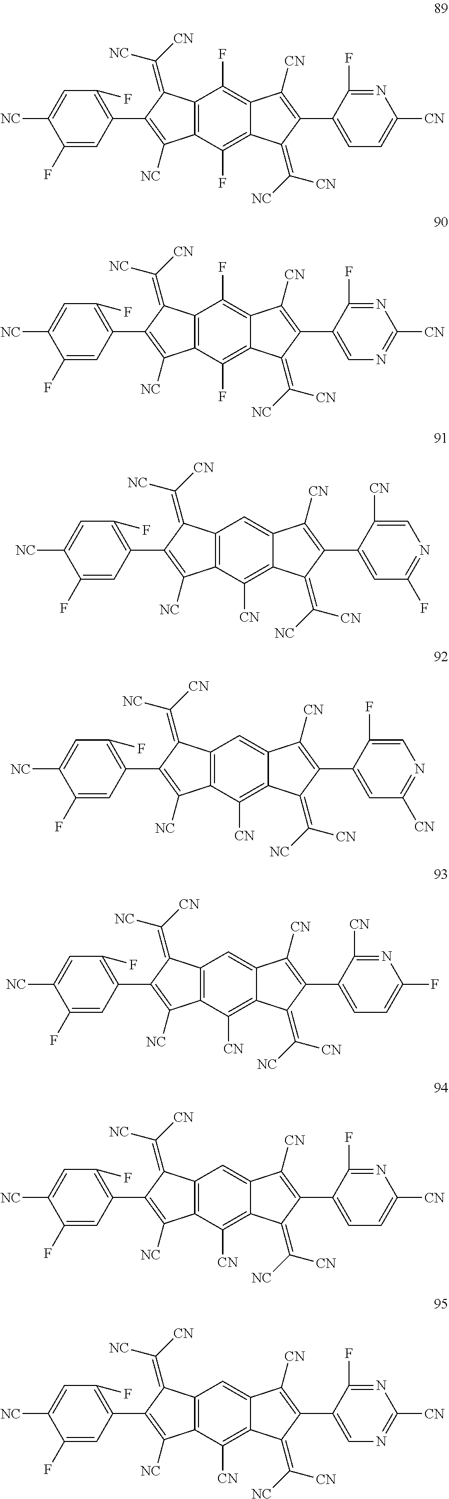

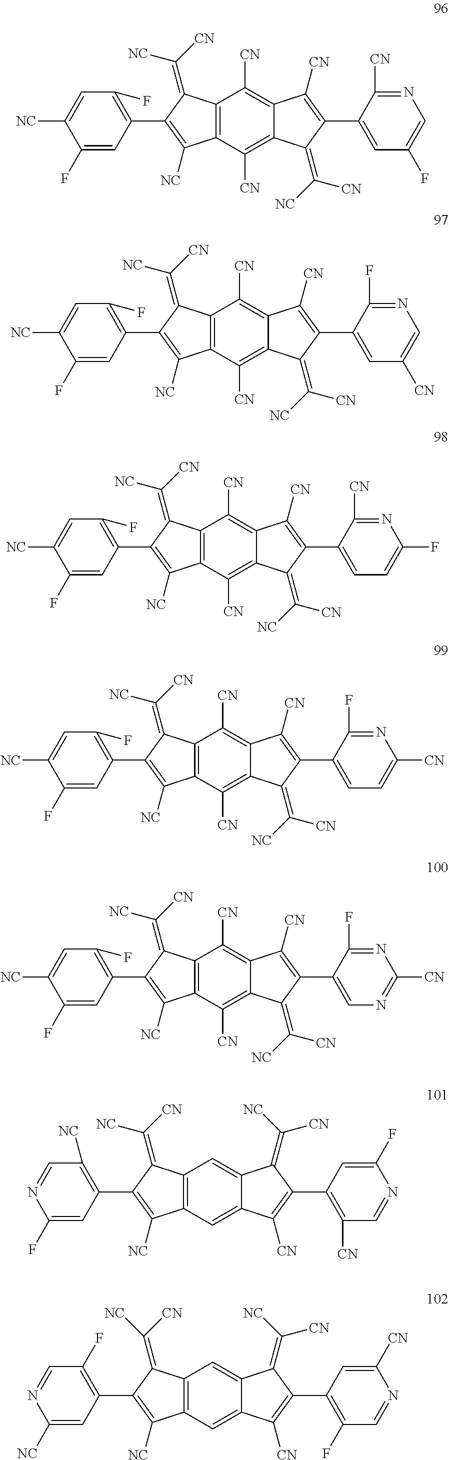

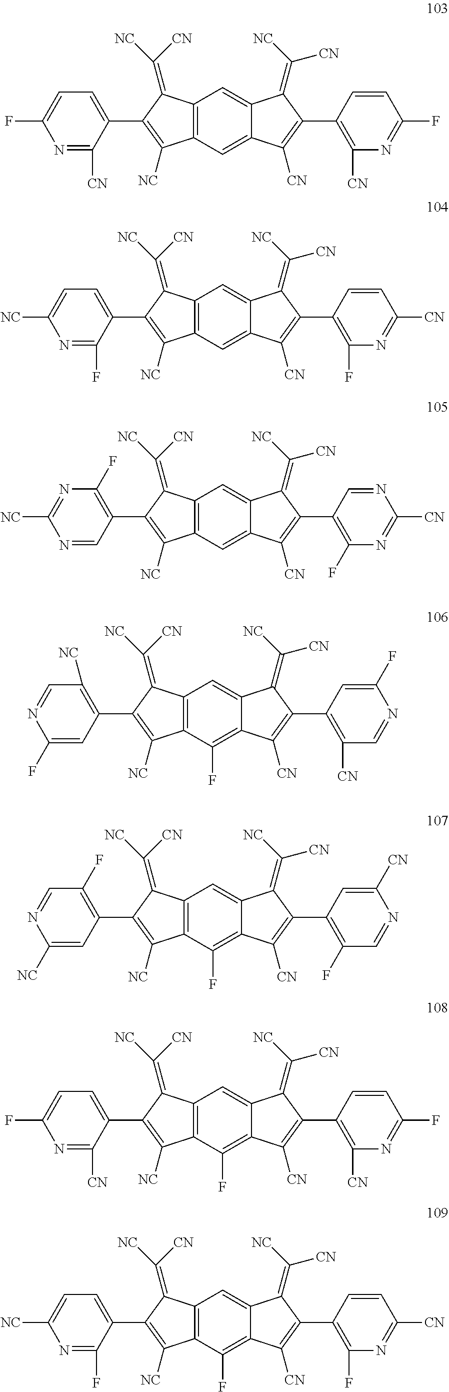

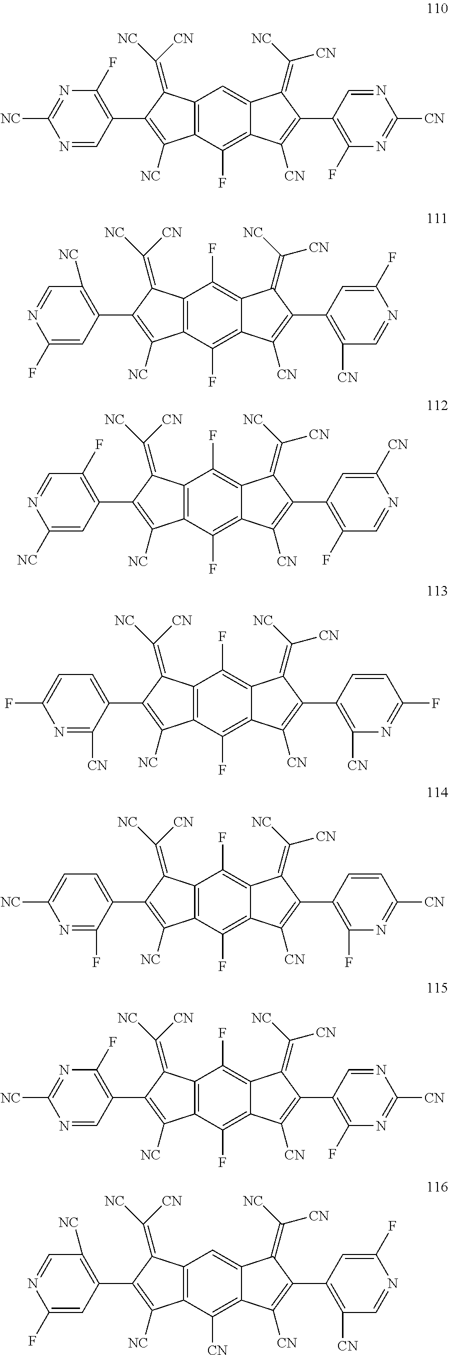

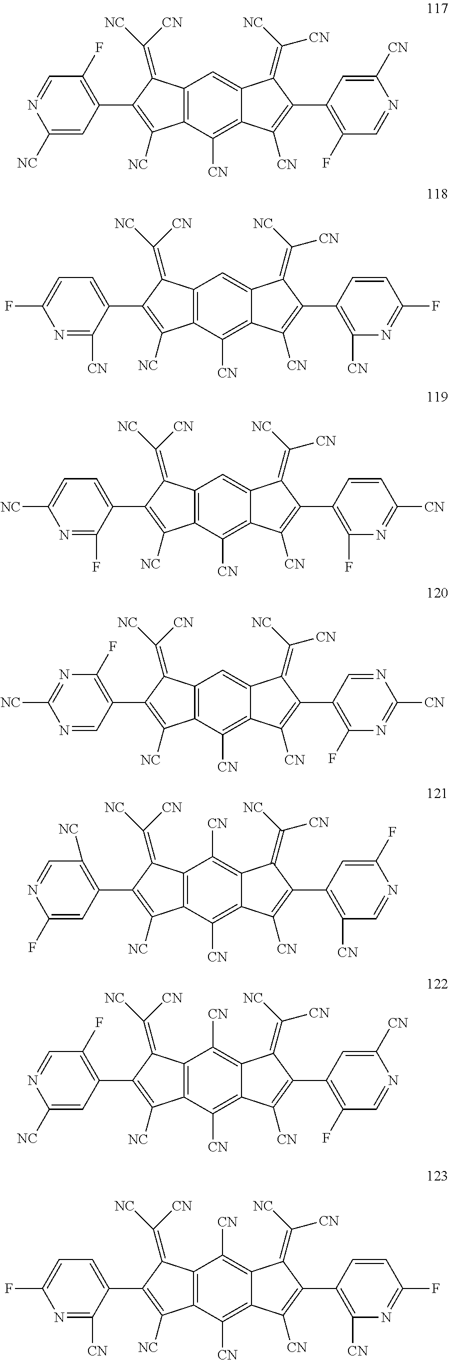

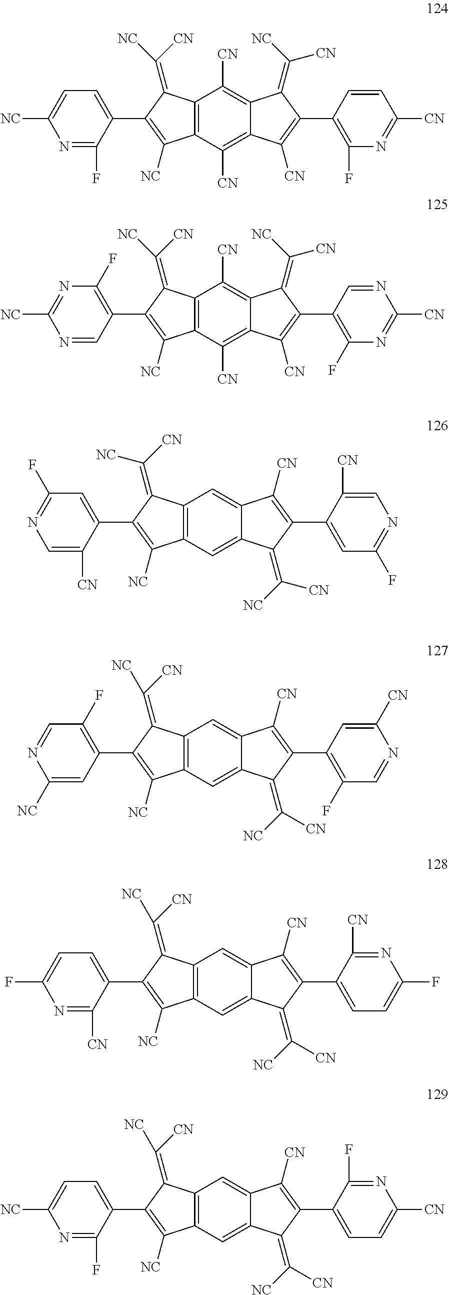

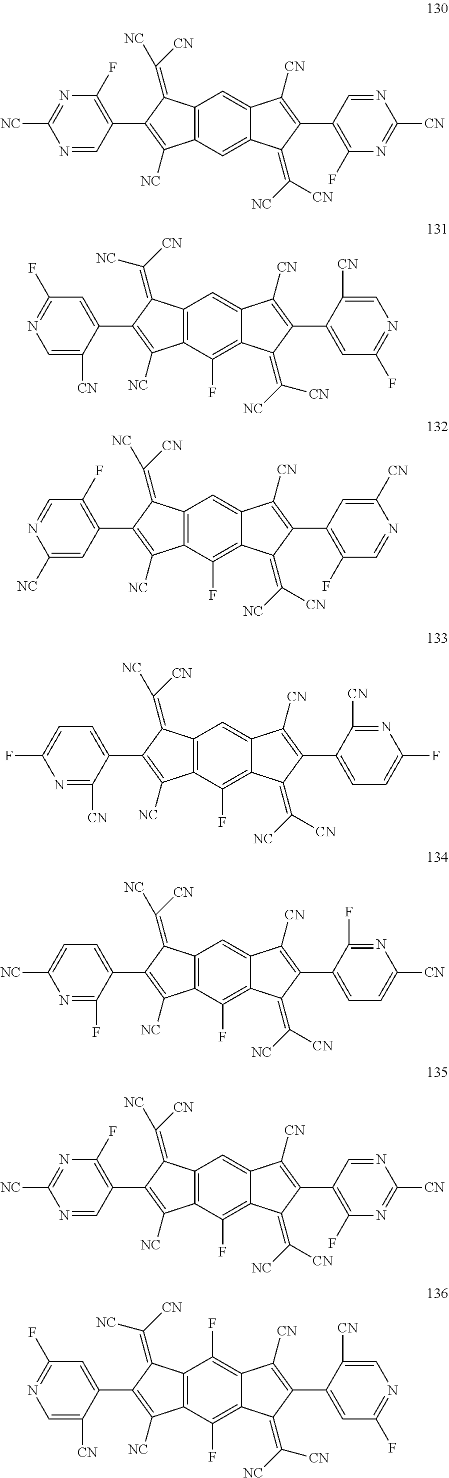

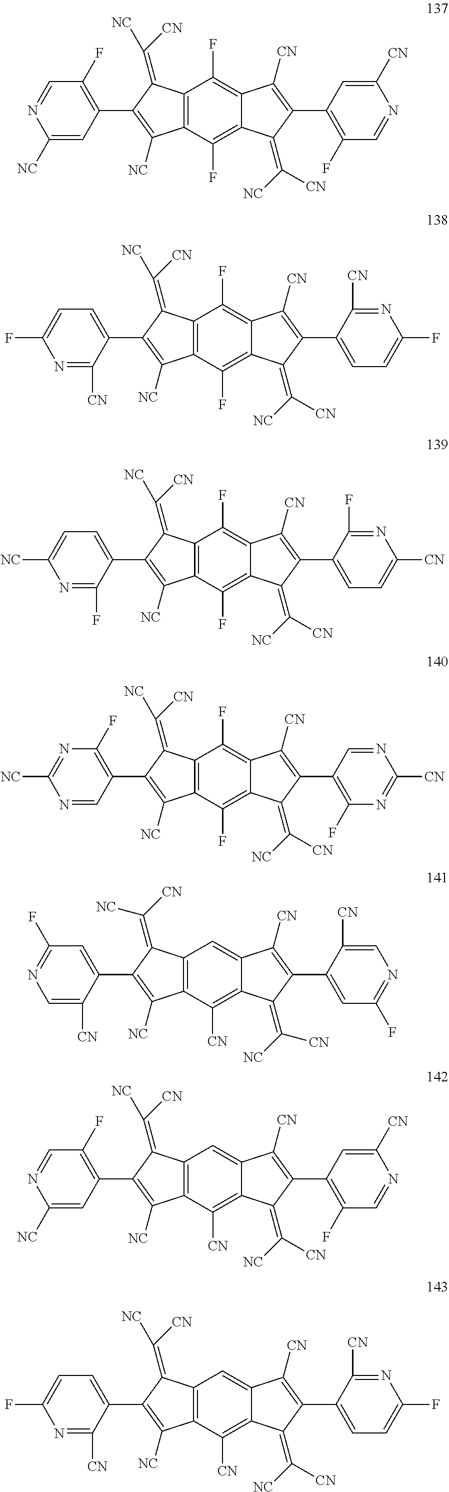

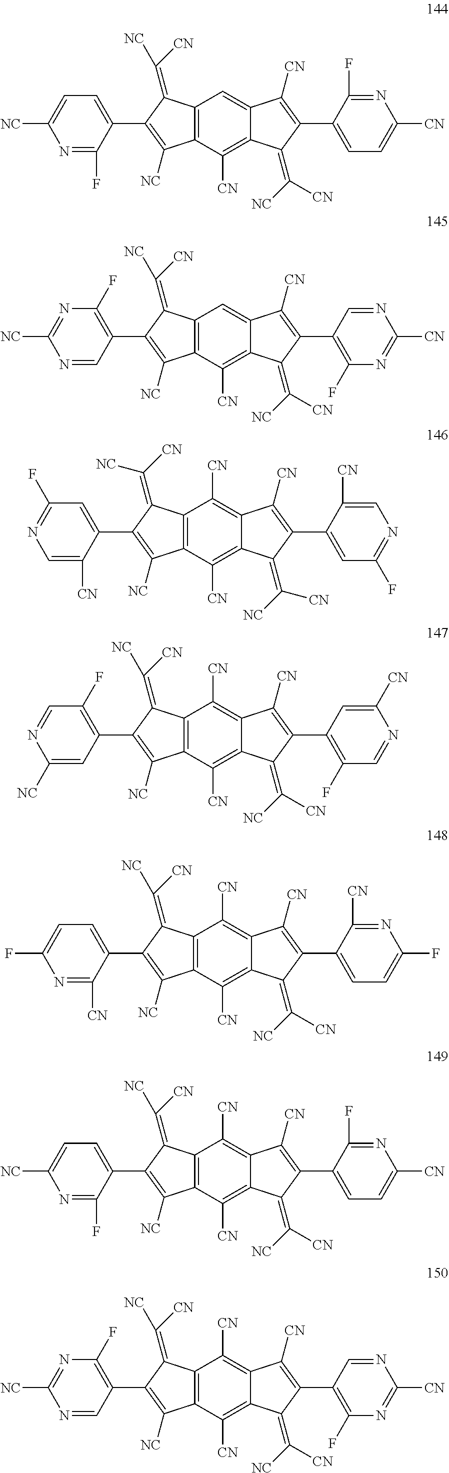

The above-described second compounds 232b, 332b, 432b, and 532b are described below.

The second compounds 232b, 332b, 432b, and 532b are represented by chemical formula 2 below.

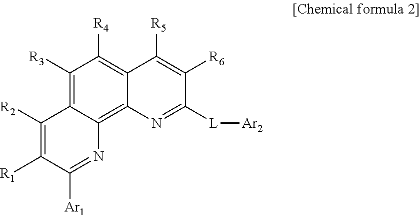

Hereinafter, chemical formula 2 is described.

R₁ to R₆ are each independently selected from the group consisting of hydrogen, deuterium, tritium, halogen, cyano group, nitro group, aryl group of C₆-C₆₀, a fluorenyl group, a heterocyclic group of C₂-C₆₀ containing at least one heteroatom of O, N, S, Si, and P, a fused ring group of an aliphatic ring of C₃-C₆₀ and an aromatic ring of C₆-C₆₀, an alkyl group of C₁-C₅₀, an alkenyl group of C₂-C₂₀, an alkynyl group of C₂-C₂₀, an alkoxy group of C₁-C₃₀, an aryloxy group of C₆-C₃₀, an alkylsilyl group of C₃-C₆₀, an arylsilyl group of C₁₈-C₆₀, and an alkylarylsilyl group of C₈-C₆₀.

When R₁ to R₆ are aryl groups, the aryl groups may be each independently an aryl group of C₆-C₆₀, an aryl group of C₆-C₅₀, or an aryl group of C₆-C₄₀.

When R₁ to R₆ are heterocyclic groups containing at least one heteroatom of O, N, S, Si, and P, the heterocyclic groups may each independently be a heterocyclic group of C₆-C₆₀, a heterocyclic group of C₆-C₅₀, or a heterocyclic group of C₆-C₄₀.

Ar₁ and Ar₂ are each independently selected from the group consisting of hydrogen, deuterium, tritium, halogen, cyano group, nitro group, aryl group of C₆-C₆₀, a fluorenyl group, a heterocyclic group of C₂-C₆₀ containing at least one heteroatom of O, N, S, Si, and P, a fused ring group of an aliphatic ring of C₃-C₆₀ and an aromatic ring of C₆-C₆₀, an alkyl group of C₁-C₅₀, an alkenyl group of C₂-C₂₀, an alkynyl group of C₂-C₂₀, an alkoxy group of C₁-C₃₀, an aryloxy group of C₆-C₃₀, an alkylsilyl group of C₃-C₆₀, an arylsilyl group of C₁₈-C₆₀, and an alkylarylsilyl group of C₈-C₆₀.

When Ar₁ and Ar₂ are aryl groups, the aryl groups may be each independently an aryl group of C₆-C₆₀, an aryl group of C₆-C₅₀, or an aryl group of C₆-C₄₀.

When Ar₁ and Ar₂ are heterocyclic groups containing at least one heteroatom of O, N, S, Si, and P, the heterocyclic groups may each independently be a heterocyclic group of C₆-C₆₀, a heterocyclic group of C₆-C₅₀, or a heterocyclic group of C₆-C₄₀.

L is selected from the group consisting of a divalent heterocyclic group of C₂-C₆₀ containing an arylene group of C₆-C₆₀, a fluorenyl group, a divalent heterocyclic group of C₂-C₆₀ containing at least one heteroatom of O, N, S, Si, and P, and a divalent fused ring group of an aliphatic ring of C₃-C₆₀ and an aromatic ring of C₆-C₆₀.

In R₁ to R₆, Ar₁, Ar₂, and L in chemical formula 2, the alkyl group, the fluorenyl group, the heterocyclic group, the fused ring group, the arylene group, the fluorenyl group, the divalent heterocyclic group, and the divalent fused ring group each may be additionally substituted with one or more substituents selected from the group consisting of deuterium, a nitro group, a cyano group, a halogen group, an amino group, an alkoxyl group of C₁-C₂₀, an alkyl group of C₁-C₂₀, an alkenyl group of C₂-C₂₀, an alkynyl group of C₂-C₂₀, an aryl group of C₆-C₂₀, an aryl group of C₆-C₂₀ substituted with deuterium, a fluorenyl group, a heterocyclic group of C₂-C₂₀, an alkylsilyl group of C₃-C₆₀, an arylsilyl group of C₁₈-C₆₀, and an alkylarylsilyl group of C₈-C₆₀.

As the organic material layers 230, 330, 430, and 530 of the organic light emitting elements 200, 300, 400, and 500 include the second compounds 232a, 332b, 432b, and 532b, the organic light emitting elements may have excellent efficiency, long lifespan or low driving voltage.

The second compounds 232b, 332b, 432b, and 532b may include two or more types of compounds having different molecular structures. For example, the second compounds 232b, 332b, 432b, and 532b may include a third compound and a fourth compound having different molecular structures.

The third compound may be represented by chemical formula 2 described above, and may be a compound in which Ar₂ is not phenanthroline in chemical formula 2. In other words, the third compound may be selected from among compounds other than compounds in which Ar₂ is phenanthroline among the compounds represented by chemical formula 2 described above.

The fourth compound may be represented by chemical formula 2 described above, and may be a compound in which Ar₂ is phenanthroline. In other words, the fourth compound may be selected from among compounds in which Ar₂ is phenanthroline among the compounds represented by chemical formula 2.

When the second compounds 232b, 332b, 432b, and 532b include the above-described third and fourth compounds, the organic light emitting element may have excellent efficiency, long lifespan, or low driving voltage.

The second compounds 232b, 332b, 432b, and 532b may be one or more among the compounds described below.

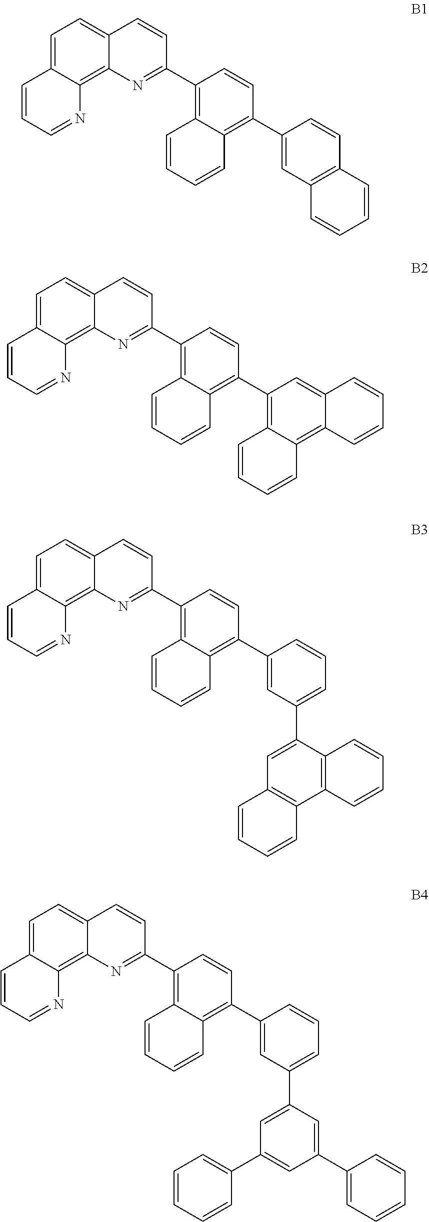 [SMILES: c1ccc2cc(-c3ccc(-c4ccc5ccc6cccnc6c5n4)c4ccccc34)ccc2c1 ; c1ccc2c(c1)cc(-c1ccc(-c3ccc4ccc5cccnc5c4n3)c3ccccc13)c1ccccc12 ; c1cc(-c2ccc(-c3ccc4ccc5cccnc5c4n3)c3ccccc23)cc(-c2cc3ccccc3c3ccccc23)c1 ; c1ccc(-c2cc(-c3ccccc3)cc(-c3cccc(-c4ccc(-c5ccc6ccc7cccnc7c6n5)c5ccccc45)c3)c2)cc1]

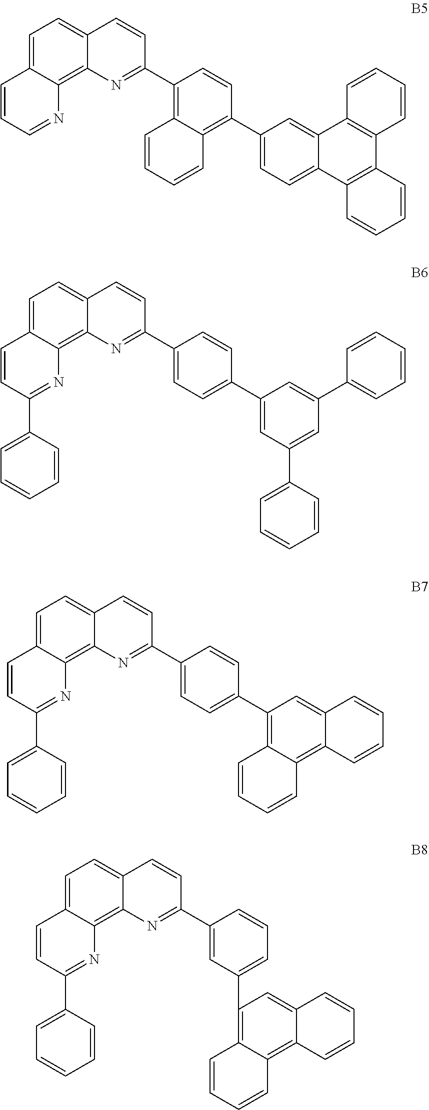 [SMILES: c1cnc2c(c1)ccc1ccc(-c3ccc(-c4ccc5c6ccccc6c6ccccc6c5c4)c4ccccc34)nc12 ; c1ccc(-c2cc(-c3ccccc3)cc(-c3ccc(-c4ccc5ccc6ccc(-c7ccccc7)nc6c5n4)cc3)c2)cc1 ; c1ccc(-c2ccc3ccc4ccc(-c5ccc(-c6cc7ccccc7c7ccccc67)cc5)nc4c3n2)cc1 ; c1ccc(-c2ccc3ccc4ccc(-c5cccc(-c6cc7ccccc7c7ccccc67)c5)nc4c3n2)cc1]

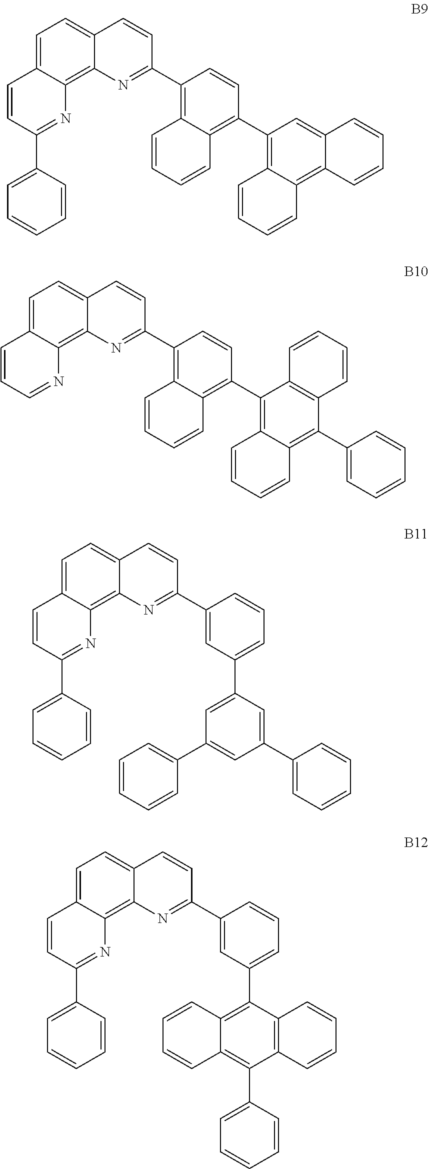

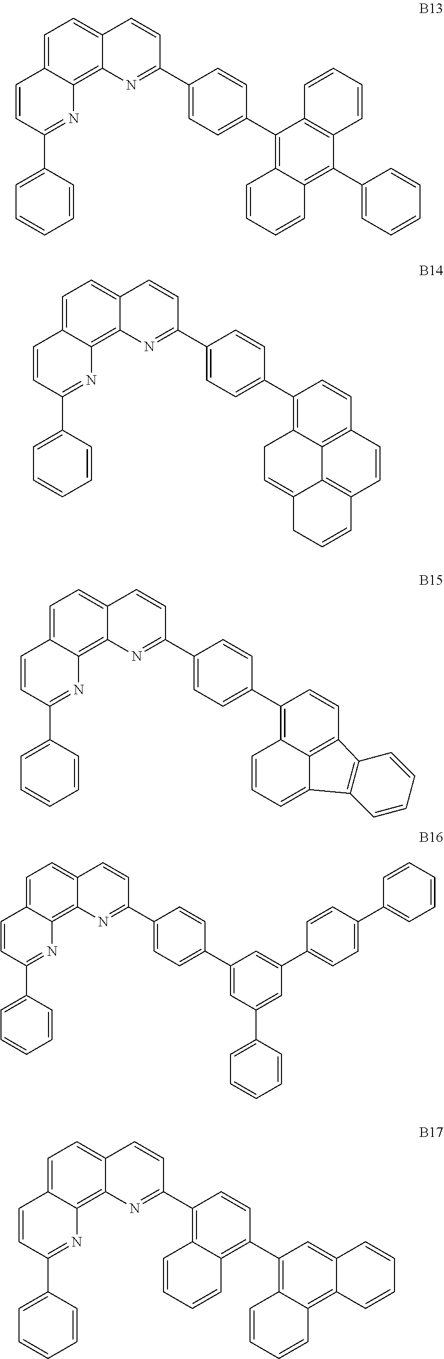 [SMILES: c1ccc(-c2ccc3ccc4ccc(-c5ccc(-c6c7ccccc7c(-c7ccccc7)c7ccccc67)cc5)nc4c3n2)cc1 ; C1=Cc2ccc3ccc(-c4ccc(-c5ccc6ccc7ccc(-c8ccccc8)nc7c6n5)cc4)c4c3c2C(=CC4)C1 ; c1ccc(-c2ccc3ccc4ccc(-c5ccc(-c6ccc7c8c(cccc68)-c6ccccc6-7)cc5)nc4c3n2)cc1 ; c1ccc(-c2ccc(-c3cc(-c4ccccc4)cc(-c4ccc(-c5ccc6ccc7ccc(-c8ccccc8)nc7c6n5)cc4)c3)cc2)cc1 ; c1ccc(-c2ccc3ccc4ccc(-c5ccc(-c6cc7ccccc7c7ccccc67)c6ccccc56)nc4c3n2)cc1]

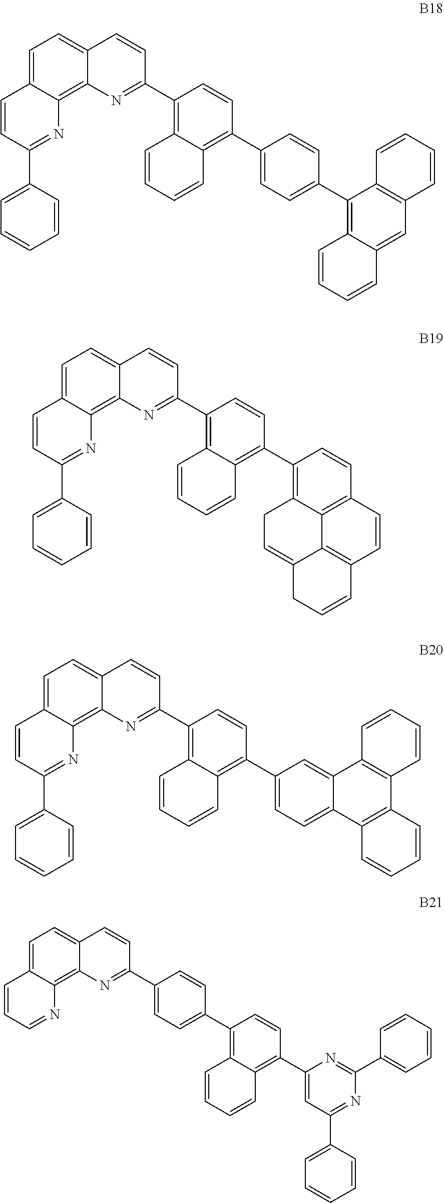 [SMILES: c1ccc(-c2ccc3ccc4ccc(-c5ccc(-c6ccc(-c7c8ccccc8cc8ccccc78)cc6)c6ccccc56)nc4c3n2)cc1 ; C1=Cc2ccc3ccc(-c4ccc(-c5ccc6ccc7ccc(-c8ccccc8)nc7c6n5)c5ccccc45)c4c3c2C(=CC4)C1 ; c1ccc(-c2ccc3ccc4ccc(-c5ccc(-c6ccc7c8ccccc8c8ccccc8c7c6)c6ccccc56)nc4c3n2)cc1 ; c1ccc(-c2cc(-c3ccc(-c4ccc(-c5ccc6ccc7cccnc7c6n5)cc4)c4ccccc34)nc(-c3ccccc3)n2)cc1]

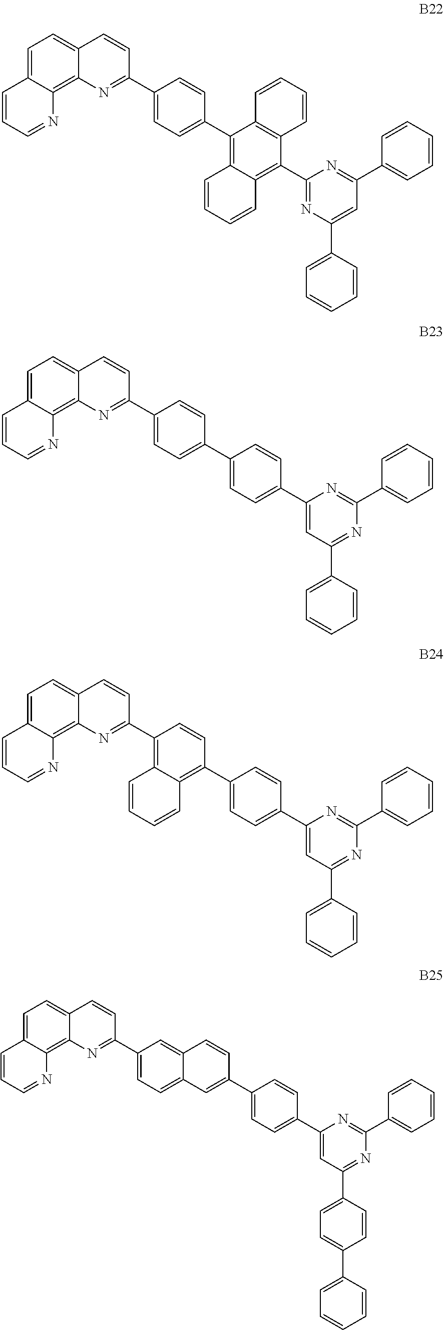 [SMILES: c1ccc(-c2cc(-c3ccccc3)nc(-c3c4ccccc4c(-c4ccc(-c5ccc6ccc7cccnc7c6n5)cc4)c4ccccc34)n2)cc1 ; c1ccc(-c2cc(-c3ccc(-c4ccc(-c5ccc6ccc7cccnc7c6n5)cc4)cc3)nc(-c3ccccc3)n2)cc1 ; c1ccc(-c2cc(-c3ccc(-c4ccc(-c5ccc6ccc7cccnc7c6n5)c5ccccc45)cc3)nc(-c3ccccc3)n2)cc1 ; c1ccc(-c2ccc(-c3cc(-c4ccc(-c5ccc6cc(-c7ccc8ccc9cccnc9c8n7)ccc6c5)cc4)nc(-c4ccccc4)n3)cc2)cc1]

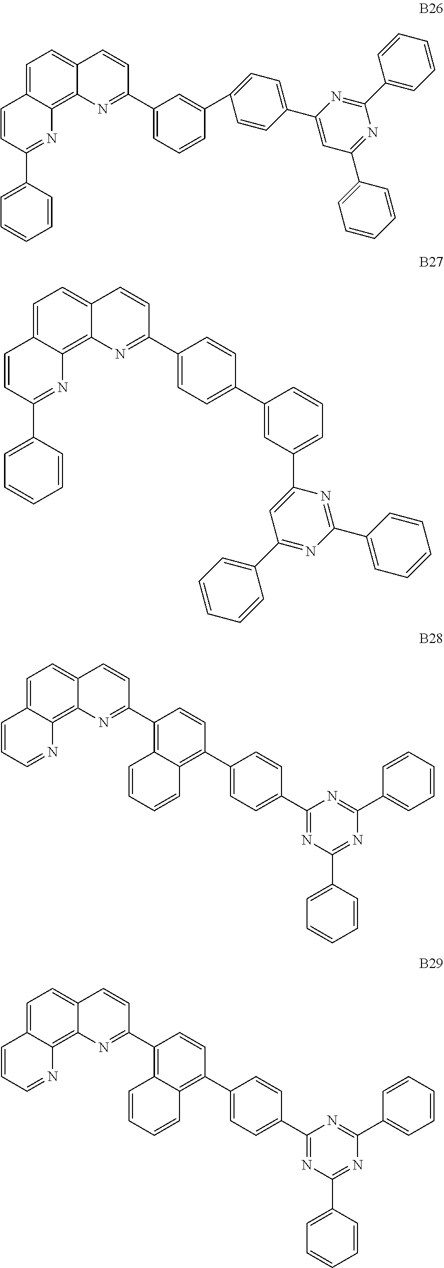 [SMILES: c1ccc(-c2cc(-c3ccc(-c4cccc(-c5ccc6ccc7ccc(-c8ccccc8)nc7c6n5)c4)cc3)nc(-c3ccccc3)n2)cc1 ; c1ccc(-c2cc(-c3cccc(-c4ccc(-c5ccc6ccc7ccc(-c8ccccc8)nc7c6n5)cc4)c3)nc(-c3ccccc3)n2)cc1 ; c1ccc(-c2nc(-c3ccccc3)nc(-c3ccc(-c4ccc(-c5ccc6ccc7cccnc7c6n5)c5ccccc45)cc3)n2)cc1 ; c1ccc(-c2nc(-c3ccccc3)nc(-c3ccc(-c4ccc(-c5ccc6ccc7cccnc7c6n5)c5ccccc45)cc3)n2)cc1]

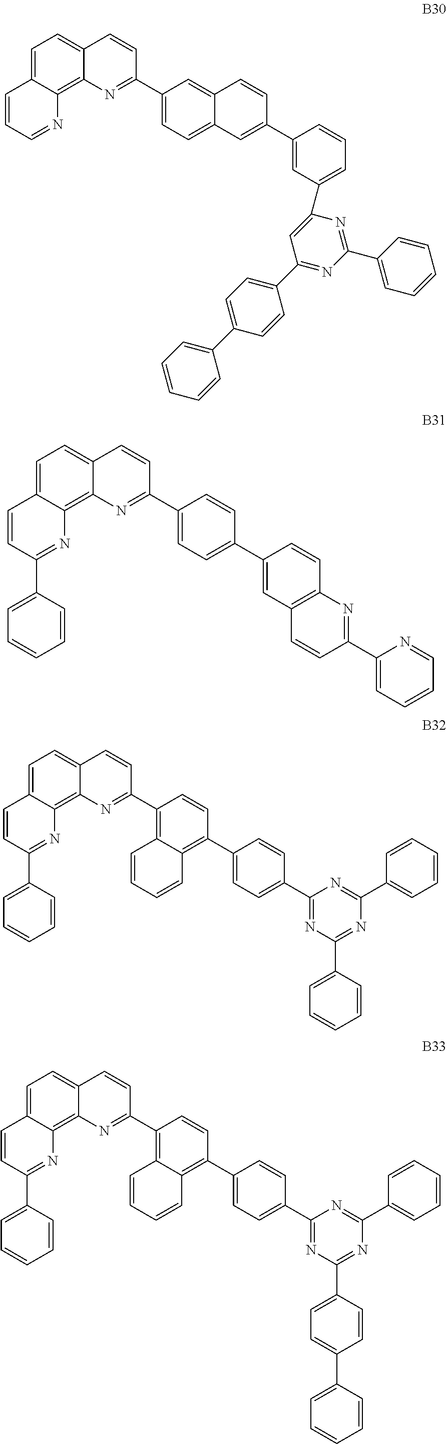 [SMILES: c1ccc(-c2ccc(-c3cc(-c4cccc(-c5ccc6cc(-c7ccc8ccc9cccnc9c8n7)ccc6c5)c4)nc(-c4ccccc4)n3)cc2)cc1 ; c1ccc(-c2ccc3ccc4ccc(-c5ccc(-c6ccc7nc(-c8ccccn8)ccc7c6)cc5)nc4c3n2)cc1 ; c1ccc(-c2ccc3ccc4ccc(-c5ccc(-c6ccc(-c7nc(-c8ccccc8)nc(-c8ccccc8)n7)cc6)c6ccccc56)nc4c3n2)cc1 ; c1ccc(-c2ccc(-c3nc(-c4ccccc4)nc(-c4ccc(-c5ccc(-c6ccc7ccc8ccc(-c9ccccc9)nc8c7n6)c6ccccc56)cc4)n3)cc2)cc1]

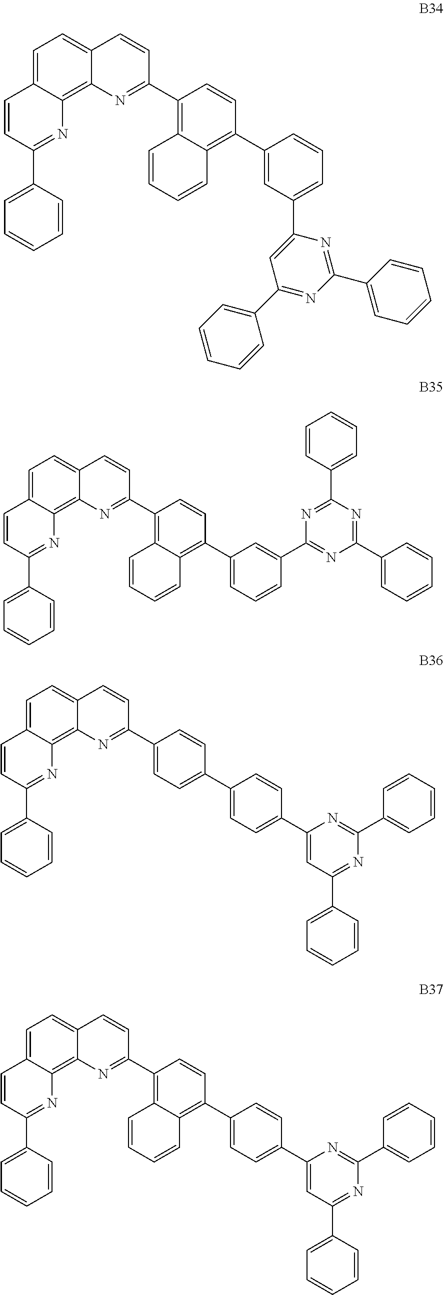 [SMILES: c1ccc(-c2cc(-c3cccc(-c4ccc(-c5ccc6ccc7ccc(-c8ccccc8)nc7c6n5)c5ccccc45)c3)nc(-c3ccccc3)n2)cc1 ; c1ccc(-c2ccc3ccc4ccc(-c5ccc(-c6cccc(-c7nc(-c8ccccc8)nc(-c8ccccc8)n7)c6)c6ccccc56)nc4c3n2)cc1 ; c1ccc(-c2cc(-c3ccc(-c4ccc(-c5ccc6ccc7ccc(-c8ccccc8)nc7c6n5)cc4)cc3)nc(-c3ccccc3)n2)cc1 ; c1ccc(-c2cc(-c3ccc(-c4ccc(-c5ccc6ccc7ccc(-c8ccccc8)nc7c6n5)c5ccccc45)cc3)nc(-c3ccccc3)n2)cc1]

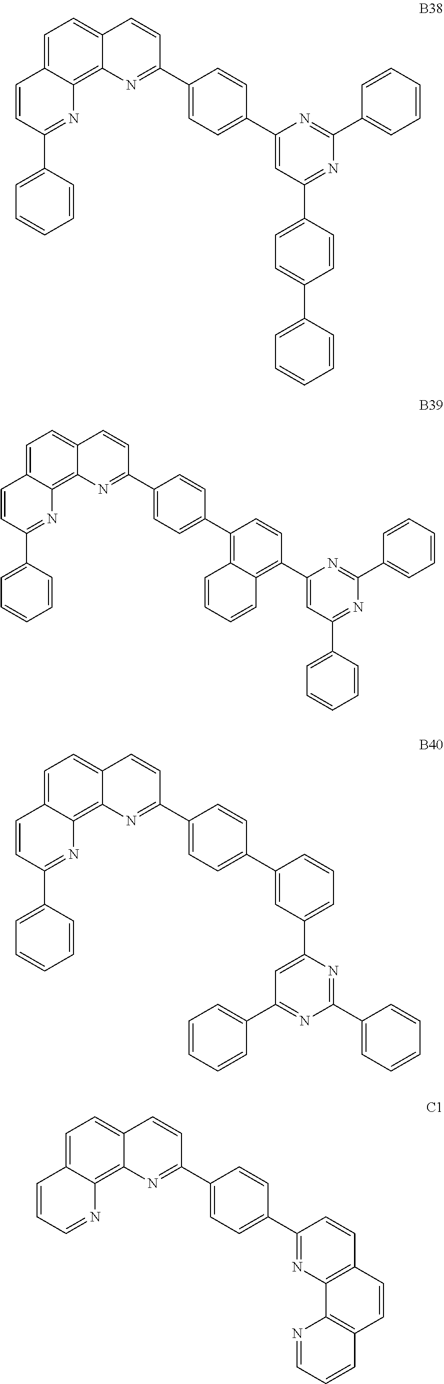 [SMILES: c1ccc(-c2ccc(-c3cc(-c4ccc(-c5ccc6ccc7ccc(-c8ccccc8)nc7c6n5)cc4)nc(-c4ccccc4)n3)cc2)cc1 ; c1ccc(-c2cc(-c3ccc(-c4ccc(-c5ccc6ccc7ccc(-c8ccccc8)nc7c6n5)cc4)c4ccccc34)nc(-c3ccccc3)n2)cc1 ; c1ccc(-c2cc(-c3cccc(-c4ccc(-c5ccc6ccc7ccc(-c8ccccc8)nc7c6n5)cc4)c3)nc(-c3ccccc3)n2)cc1 ; c1cnc2c(c1)ccc1ccc(-c3ccc(-c4ccc5ccc6cccnc6c5n4)cc3)nc12]

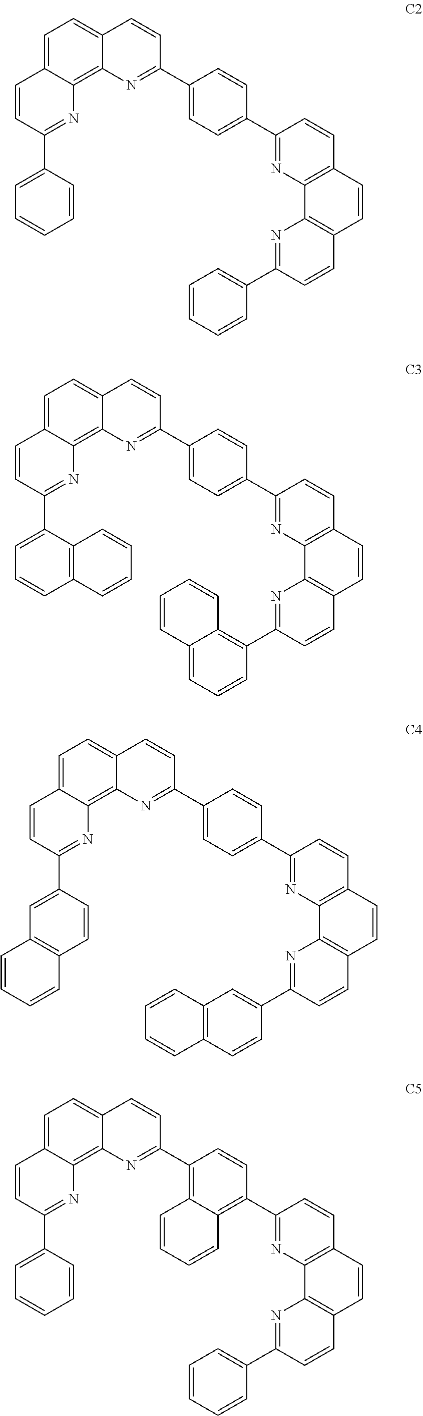 [SMILES: c1ccc(-c2ccc3ccc4ccc(-c5ccc(-c6ccc7ccc8ccc(-c9ccccc9)nc8c7n6)cc5)nc4c3n2)cc1 ; c1ccc2c(-c3ccc4ccc5ccc(-c6ccc(-c7ccc8ccc9ccc(-c%10cccc%11ccccc%10%11)nc9c8n7)cc6)nc5c4n3)cccc2c1 ; c1ccc2cc(-c3ccc4ccc5ccc(-c6ccc(-c7ccc8ccc9ccc(-c%10ccc%11ccccc%11c%10)nc9c8n7)cc6)nc5c4n3)ccc2c1 ; c1ccc(-c2ccc3ccc4ccc(-c5ccc(-c6ccc7ccc8ccc(-c9ccccc9)nc8c7n6)c6ccccc56)nc4c3n2)cc1]

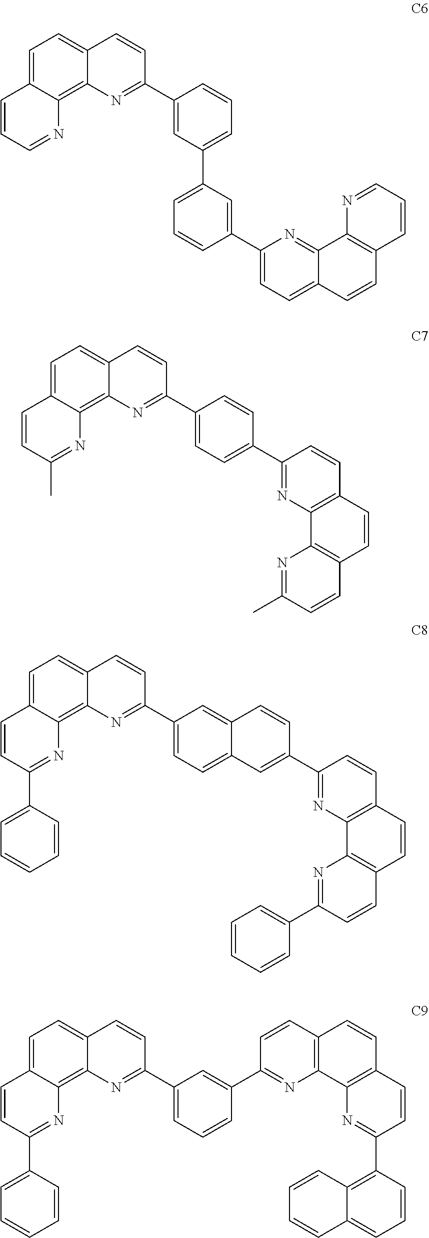 [SMILES: c1cc(-c2cccc(-c3ccc4ccc5cccnc5c4n3)c2)cc(-c2ccc3ccc4cccnc4c3n2)c1 ; Cc1ccc2ccc3ccc(-c4ccc(-c5ccc6ccc7ccc(C)nc7c6n5)cc4)nc3c2n1 ; c1ccc(-c2ccc3ccc4ccc(-c5ccc6cc(-c7ccc8ccc9ccc(-c%10ccccc%10)nc9c8n7)ccc6c5)nc4c3n2)cc1 ; c1ccc(-c2ccc3ccc4ccc(-c5cccc(-c6ccc7ccc8ccc(-c9cccc%10ccccc9%10)nc8c7n6)c5)nc4c3n2)cc1]

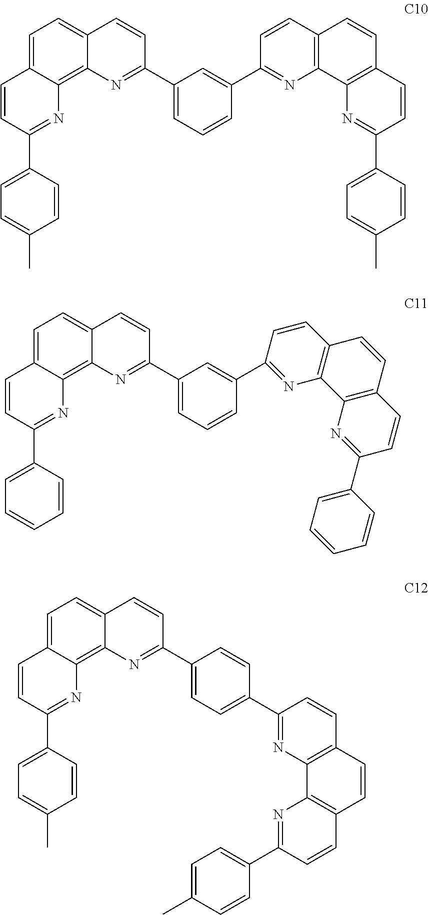 [SMILES: Cc1ccc(-c2ccc3ccc4ccc(-c5cccc(-c6ccc7ccc8ccc(-c9ccc(C)cc9)nc8c7n6)c5)nc4c3n2)cc1 ; c1ccc(-c2ccc3ccc4ccc(-c5cccc(-c6ccc7ccc8ccc(-c9ccccc9)nc8c7n6)c5)nc4c3n2)cc1 ; Cc1ccc(-c2ccc3ccc4ccc(-c5ccc(-c6ccc7ccc8ccc(-c9ccc(C)cc9)nc8c7n6)cc5)nc4c3n2)cc1]

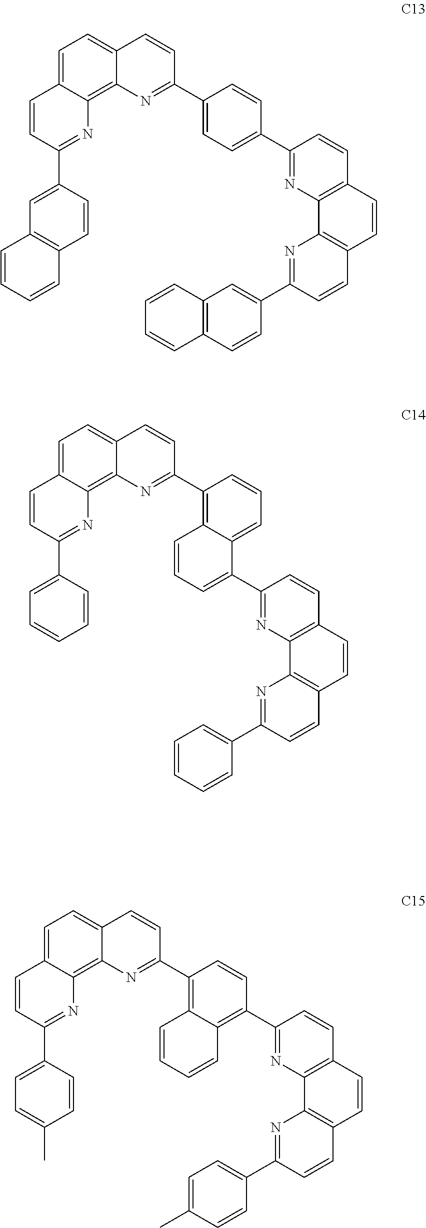 [SMILES: c1ccc2cc(-c3ccc4ccc5ccc(-c6ccc(-c7ccc8ccc9ccc(-c%10ccc%11ccccc%11c%10)nc9c8n7)cc6)nc5c4n3)ccc2c1 ; c1ccc(-c2ccc3ccc4ccc(-c5cccc6c(-c7ccc8ccc9ccc(-c%10ccccc%10)nc9c8n7)cccc56)nc4c3n2)cc1 ; Cc1ccc(-c2ccc3ccc4ccc(-c5ccc(-c6ccc7ccc8ccc(-c9ccc(C)cc9)nc8c7n6)c6ccccc56)nc4c3n2)cc1]

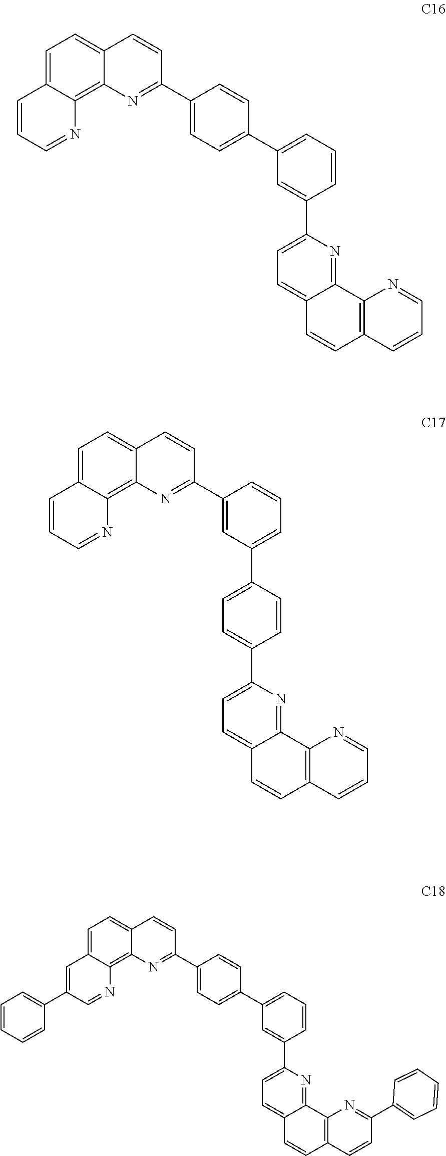 [SMILES: c1cc(-c2ccc(-c3ccc4ccc5cccnc5c4n3)cc2)cc(-c2ccc3ccc4cccnc4c3n2)c1 ; c1cc(-c2ccc(-c3ccc4ccc5cccnc5c4n3)cc2)cc(-c2ccc3ccc4cccnc4c3n2)c1 ; c1ccc(-c2cnc3c(ccc4ccc(-c5ccc(-c6cccc(-c7ccc8ccc9ccc(-c%10ccccc%10)nc9c8n7)c6)cc5)nc43)c2)cc1]

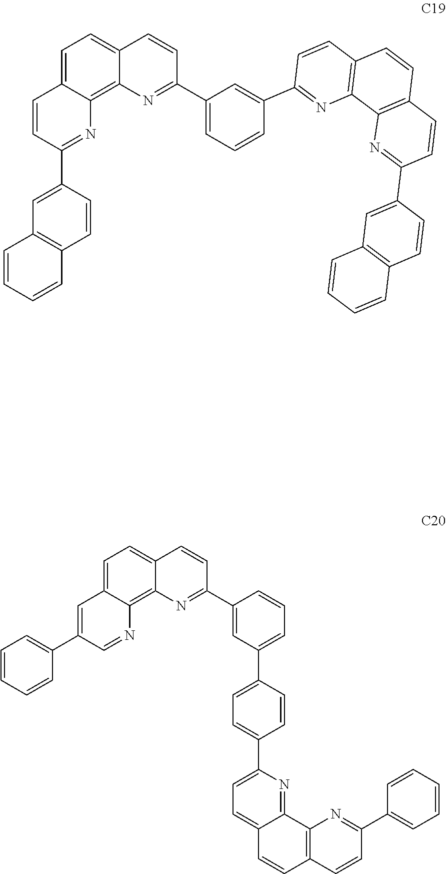 [SMILES: c1cc(-c2ccc3ccc4ccc(-c5ccc6ccccc6c5)nc4c3n2)cc(-c2ccc3ccc4ccc(-c5ccc6ccccc6c5)nc4c3n2)c1 ; c1ccc(-c2cnc3c(ccc4ccc(-c5cccc(-c6ccc(-c7ccc8ccc9ccc(-c%10ccccc%10)nc9c8n7)cc6)c5)nc43)c2)cc1]

For example, the third compound may be one or more of compounds B1 to B40 described above. Further, the fourth compound may be one or more of the above compounds C1 to C20.

Other embodiments of the disclosure may provide a display device. The display device may include the organic light emitting elements 200, 300, 400, and 500 described above with reference to FIGS. 1 to 6.

Embodiments of the disclosure described above are briefly described below.

An organic light emitting element 200, 300, 400, or 500 according to embodiments of the disclosure may comprise a first electrode 210, 310, 410, or 510, a second electrode 220, 320, 420, or 520, and an organic material layer 230, 330, 430, or 530. The organic material layer 230, 330, 430, or 530 may include a first compound 232a, 332a, 432a, or 532a represented by chemical formula 1 described above. The organic material layer 230, 330, 430, or 530 may include a second compound 232b, 332b, 432b, or 532b represented by chemical formula 2 described above.

The organic material layer 330, 430, or 530 may include a first light emitting layer 331, 431, or 531 and a first layer 332, 432, or 532. The first layer 332, 432, or 532 may include a first compound 332a, 432a, or 532a and a second compound 332b, 432b, or 532b.

The organic material layer 330, 430, or 530 may include a second light emitting layer 333, 433, or 533, and the first layer 332, 432, or 532 may be positioned between the first light emitting layer 331, 431, or 531 and the second light emitting layer 333, 433, or 533.

The first layer 332 or 432 may be a charge generation layer. The first layer 432 or 532 may include an n-type charge generation layer 4321 and a p-type charge generation layer 4322. The n-type charge generation layer 4321 may include the second compound 432b. The p-type charge generation layer 4322 may include the first compound 432a.

The first electrode 410 may be an anode electrode and the second electrode 420 may be a cathode electrode. The n-type charge generation layer 4321 may be positioned on the first electrode 410, and the p-type charge generation layer 4322 may be positioned on the n-type charge generation layer 4321.

The organic material layer 530 may include a third light emitting layer 535 and a charge generation layer 534 positioned between the second light emitting layer 533 and the third light emitting layer 535.

The display device 100 according to embodiments of the disclosure includes an organic light emitting element 200, 300, 400, or 500.

An example of manufacturing an organic light emitting element according to embodiments of the disclosure are described below in detail with reference to embodiments thereof, but embodiments of the disclosure are not limited to the following embodiments.

### COMPOUND SYNTHESIS EXAMPLES

### Preparation Example 1-1. Synthesis of Compound 1-A

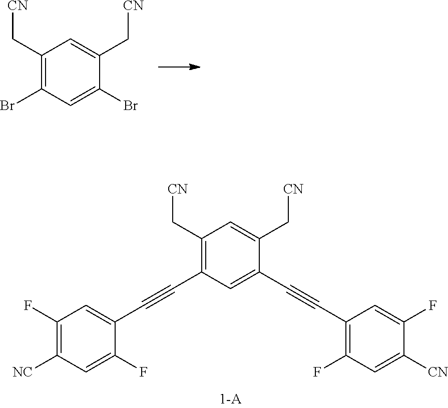 [SMILES: N#CCc1cc(CC#N)c(Br)cc1Br ; N#CCc1cc(CC#N)c(C#Cc2cc(F)c(C#N)cc2F)cc1C#Cc1cc(F)c(C#N)cc1F]

78.5 g (250.0 mmol) of 2,2′-(4,6-dibromo-1,3-phenylene) diacetonitrile, 1.2 L of toluene, 20.0 mmol of copper iodide, 20.0 mmol of tetrakistriphenylphosphine palladium, 1250.0 mmol of diisopropylamine, and 625.0 mmol of 4-ethynyl-2.5-difluorobenzonitrile were mixed, heated to 100° C., and stirred for 2 hours. After the reaction, 1.0 L of the solvent was distilled, and the reaction solution returned to room temperature was filtered to obtain a solid. After dissolving the solid in chloroform and extracting with water, magnesium sulfate and acid clay were added and stirred for one hour. After filtering the stirred solution, the solvent was distilled off again, and recrystallization was performed twice with tetrahydrofuran/ethanol to obtain 26.3 g of compound 1-A (yield 22%, MS[M+H]=479).

### Preparation Example 1-2. Synthesis of Compound 1-B

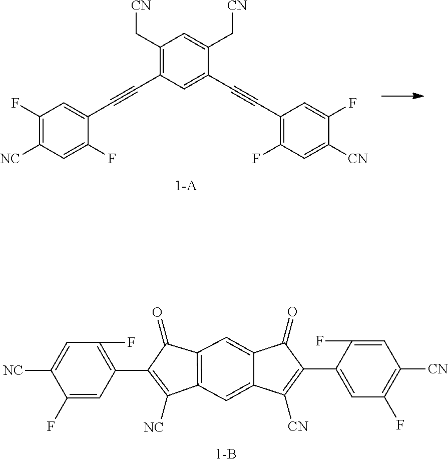 [SMILES: N#CCc1cc(CC#N)c(C#Cc2cc(F)c(C#N)cc2F)cc1C#Cc1cc(F)c(C#N)cc1F ; N#Cc1cc(F)c(-c2c(C#N)c3cc4c(C#N)c(-c5cc(F)c(C#N)cc5F)c(=O)c4cc3c2=O)cc1F]

26.3 g (55.0 mmol) of 1-A, 550.0 ml of 1,4-dioxane, 330.0 mmol of diphenyl sulfoxide, 11.0 mmol of copper bromide (II), and 11.0 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 3.9 g of compound 1-B (yield 14%, MS [M+H]=507).

### Preparation Example 1-3. Synthesis of Compound 1

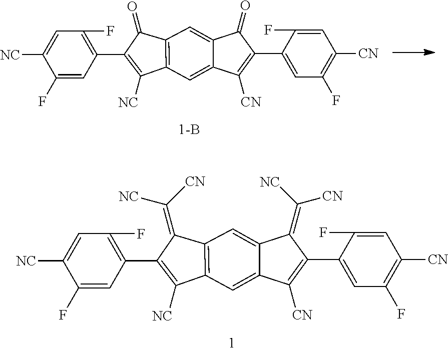 [SMILES: N#Cc1cc(F)c(-c2c(C#N)c3cc4c(C#N)c(-c5cc(F)c(C#N)cc5F)c(=O)c4cc3c2=O)cc1F ; N#CC(C#N)=C1C(c2cc(F)c(C#N)cc2F)=C(C#N)c2cc3c(cc21)C(=C(C#N)C#N)C(c1cc(F)c(C#N)cc1F)=C3C#N]

3.9 g (7.7 mmol) of 1-B, 154.0 mL of dichloromethane, and 46.2 mmol of malononitrile were added and cooled to 0° C. After slowly adding 38.5 mmol of titanium chloride (IV) at 0° C., the mixture was stirred for 1 hour while maintaining the temperature at 0° C. 57.8 mmol of pyridine was dissolved in 48.0 ml of dichloromethane, and then added slowly to the mixture at 0° C., and then the mixture was stirred for one hour while maintaining the temperature. After the reaction was complete, 77.0 mmol of acetic acid was added and the reaction solution obtained was stirred for an additional 30 minutes. After the reaction solution was extracted with water, the organic layer was reverse-precipitated in hexane to obtain a solid. The obtained solid was extracted with acetonitrile and filtered to obtain a filtrate. After adding magnesium sulfate and acid clay to the obtained filtrate, and the solution obtained was stirred for 30 minutes. 4.6 g

After filtering the solution, it was recrystallized with acetonitrile/toluene and washed with toluene. The obtained solid was recrystallized again using acetonitrile/tert-butylmethylether and purified by sublimation, obtaining 0.8 g of compound 1 (yield 18%, MS[M+H]=603).

### Preparation Example 2-1. Synthesis of Compound 26-A

78.5 g (250 mmol) of 2,5-dibromobenzene-1,4-diacetonitrile, 900.0 mL of toluene, 20.0 mmol of copper iodide, 20.0 mmol of tetrakistriphenylphosphine palladium, 1250.0 mmol of diisopropylamine, and 625.0 mmol of 4-ethynyl-2.5-difluorobenzonitrile were mixed, heated to 100° C., and stirred for 2 hours. After the reaction was complete, 800.0 ml of the solvent was distilled off, and the reaction solution returned to room temperature was filtered to obtain a solid. After dissolving the solid in chloroform and extracting with water, magnesium sulfate and acid clay were added and the solution obtained was stirred for one hour. After filtering the stirred solution, the solvent was distilled off again, and recrystallization was performed twice with tetrahydrofuran/ethanol to obtain 16.8 g of compound 26-A (yield 14%, MS[M+H]=479).

### Preparation Example 3-2. Synthesis of Compound 26-B

16.8 g (35.0 mmol) of 26-A, 220.0 ml of 1,4-dioxane, 210.0 mmol of diphenyl sulfoxide, 7.0 mmol of copper bromide (II), and 7.0 mmol of palladium acetate were mixed, heated to 100° C., and stirred for 5 hours. After the reaction was complete, the solvent was distilled off, the residue was dissolved in chloroform, acid clay was added, and the solution obtained was stirred for one hour. After filtering the stirred solution, the solvent was distilled off again and reverse-precipitation was performed using hexane to obtain a solid. The obtained solid was recrystallized with tetrahydrofuran/hexane and filtered to obtain 4.1 g of compound 26-B (yield 23%, MS [M+H]=507).

### Preparation Example 2-3. Synthesis of Compound 26

4.1 g (8.5 mmol) of 26-B, 130.0 mL of dichloromethane, and 59.5 mmol of malononitrile were added and cooled to 0° C. After slowly adding 42.5 mmol of titanium chloride (IV) at 0° C., the mixture was stirred for 1 hour while maintaining the temperature at 0° C. 59.5 mmol of pyridine was dissolved in 41.0 ml of dichloromethane, and then added slowly at 0° C., and then it was stirred for one hour while maintaining the temperature. After the reaction was complete, 42.5 mmol of acetic acid was added and the reaction solution obtained was stirred for an additional 30 minutes. After the reaction solution was extracted with water, the organic layer was reverse-precipitated in hexane to obtain a solid. After filtering the solution, it was recrystallized with acetonitrile/toluene and washed with toluene. The obtained solid was recrystallized again using acetonitrile/tert-butylmethylether and purified by sublimation, obtaining 0.8 g of compound 26 (yield 17%, MS[M+H]=603).

### Preparation example 3-1. Synthesis of compound 52-A

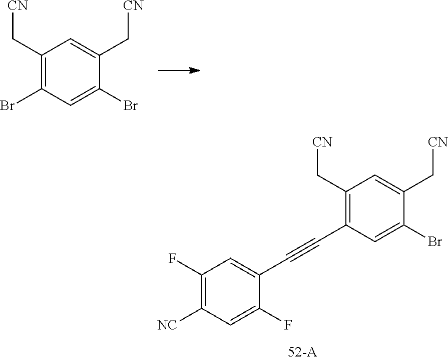 [SMILES: N#CCc1cc(CC#N)c(Br)cc1Br ; N#CCc1cc(CC#N)c(C#Cc2cc(F)c(C#N)cc2F)cc1Br]

240.6 g (766.4 mmol) of 2,2′-(4,6-dibromo-1,3-phenylene) diacetonitrile, 4.0 L of toluene, 153.28 mmol of copper iodide, 153.28 mmol of tetrakistriphenylphosphine palladium, 3832.0 mmol of diisopropylamine, and 766.4 mmol of 4-ethynyl-2.5-difluorobenzonitrile were mixed, and heated to 100° C. After the reaction, 3.6 L of the solvent was distilled, and the reaction solution returned to room temperature was filtered to obtain a solid. After dissolving the solid in chloroform and extracting with water, magnesium sulfate and acid clay were added and stirred for one hour. After filtering the stirred solution, the solvent was distilled off again, and recrystallization was performed twice with ethanol to obtain 121.4 g of compound 52-A (yield 40%, MS[M+H]=397).

### Preparation Example 3-2. Synthesis of Compound 52-B

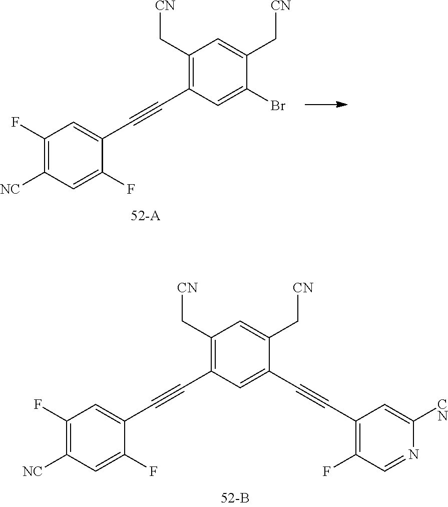 [SMILES: N#CCc1cc(CC#N)c(C#Cc2cc(F)c(C#N)cc2F)cc1Br ; N#CCc1cc(CC#N)c(C#Cc2cc(F)c(C#N)cc2F)cc1C#Cc1cc(C#N)ncc1F]

101.4 g (256.0 mmol) of 52-A, 1.2 L of toluene, 51.2 mmol of copper iodide, 51.2 mmol of tetrakistriphenylphosphine palladium, 1280 mmol of diisopropylamine, and 256.0 mmol of 4-ethinyl-6-fluoropyridine-3-carbonitrile were mixed, heated to 100° C., and stirred for 2 hours. After the reaction, 1.0 L of the solvent was distilled, and the reaction solution returned to room temperature was filtered to obtain a solid. After dissolving the solid in chloroform and extracting with water, magnesium sulfate and acid clay were added and stirred for one hour. After filtering the stirred solution, the solvent was distilled off again, and recrystallization was performed twice with tetrahydrofuran/ethanol to obtain 29.5 g of compound 52-B (yield 25%, MS[M+H]=462).

### Preparation Example 3-3. Synthesis of Compound 52-C

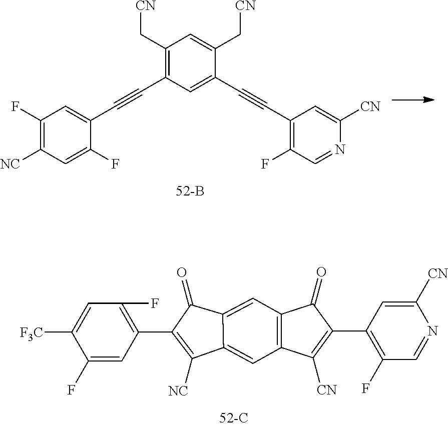 [SMILES: N#CCc1cc(CC#N)c(C#Cc2cc(F)c(C#N)cc2F)cc1C#Cc1cc(C#N)ncc1F ; N#Cc1cc(-c2c(C#N)c3cc4c(C#N)c(-c5cc(F)c(C(F)(F)F)cc5F)c(=O)c4cc3c2=O)c(F)cn1]

22.6 g (49.1 mmol) of 52-B, 300 mL of 1,4-dioxane, 294.6 mmol of diphenyl sulfoxide, 9.82 mmol of copper bromide (II), and 9.82 mmol of palladium acetate were mixed, heated to 100° C., and stirred for 5 hours. After the reaction, the solvent was distilled off, dissolved in chloroform, acid clay was added, and stirred for one hour. After filtering the stirred solution, the solvent was distilled off again and reverse-precipitated was performed using hexane to obtain a solid. The obtained solid was recrystallized with tetrahydrofuran/hexane and filtered to obtain 4.8 g of compound 52-C (yield 20%, MS [M+H]=490).

### Preparation Example 3-4. Synthesis of Compound 52

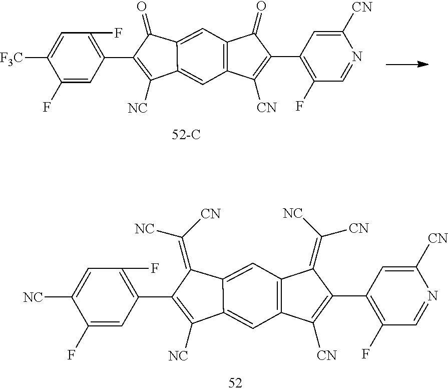 [SMILES: N#Cc1cc(-c2c(C#N)c3cc4c(C#N)c(-c5cc(F)c(C(F)(F)F)cc5F)c(=O)c4cc3c2=O)c(F)cn1 ; N#CC(C#N)=C1C(c2cc(C#N)ncc2F)=C(C#N)c2cc3c(cc21)C(=C(C#N)C#N)C(c1cc(F)c(C#N)cc1F)=C3C#N]

3.7 g (7.6 mmol) of 52-C, 120 mL of dichloromethane, and 53.2 mmol of malononitrile were added and cooled to 0° C. After slowly adding 38.0 mmol of titanium chloride (IV)° C., it was stirred for 1 hour while remaining at 0° C. 53.2 mmol of pyridine was dissolved in 40 mL of dichloromethane, and then added slowly at 0° C., and then it was stirred for one hour while maintaining the temperature. After the reaction was complete, 53.2 mmol of acetic acid was added and stirred for an additional 30 minutes. After the reaction solution was extracted with water, the organic layer was reverse-precipitated in hexane to obtain a solid. The obtained solid was filtered through acetonitrile, and magnesium sulfate and acid clay were added, followed by stirring for 30 minutes. After filtering the solution, it was recrystallized with acetonitrile/toluene and washed with toluene. The obtained solid was recrystallized again using acetonitrile/tert-butylmethylether and purified by sublimation, obtaining 0.8 g of compound 52 (yield 18%, MS[M+H]=586).

### Preparation Example 4-1. Synthesis of Compound 102-A

74.3 g (236.5 mmol) of 2,2′-(4,6-dibromo-1,3-phenylene) diacetonitrile, 1.0 L of toluene, 47.3 mmol of copper iodide, 47.3 mmol of tetrakistriphenylphosphine palladium, 1182.5 mmol of diisopropylamine, and 709.5 mmol of 4-ethinyl-5-fluoropyridine-2-carbonitrile were mixed, heated to 100° C., and stirred for 2 hours. After the reaction, 900 ml of the solvent was distilled, and the reaction solution returned to room temperature was filtered to obtain a solid. After dissolving the solid in chloroform and extracting with water, magnesium sulfate and acid clay were added and stirred for one hour. After filtering the stirred solution, the solvent was distilled off again, and recrystallization was performed twice with tetrahydrofuran/ethanol to obtain 31.5 g of compound 102-A (yield 30%, MS[M+H]=445).

### Preparation Example 4-2. Synthesis of Compound 102-B

29.8 g (67.1 mmol) of 102-A, 400 mL of 1,4-dioxane, 402.6 mmol of diphenyl sulfoxide, 13.4 mmol of copper bromide (II), and 13.4 mmol of palladium acetate were mixed, heated to 100° C., and stirred for 5 hours. After the reaction, the solvent was distilled off, dissolved in chloroform, acid clay was added, and stirred for one hour. After filtering the stirred solution, the solvent was distilled off again and reverse-precipitated was performed using hexane to obtain a solid. The obtained solid was recrystallized with tetrahydrofuran/hexane and filtered to obtain 5.8 g of compound 102-B (yield 18%, MS [M+H]=473).

### Preparation Example 4-3. Synthesis of Compound 102

4.2 g (8.8 mmol) of 102-B, 130 ml of dichloromethane, and 61.6 mmol of malononitrile were added and cooled to 0° C. After slowly adding 44.0 mmol of titanium chloride (IV), it was stirred for 1 hour while remaining at 0° C. 61.6 mmol of pyridine was dissolved in 40 ml of dichloromethane, and then added slowly at 0° C., and then it was stirred for one hour while maintaining the temperature. After the reaction was complete, 61.6 mmol of acetic acid was added and stirred for an additional 30 minutes. After the reaction solution was extracted with water, the organic layer was reverse-precipitated in hexane to obtain a solid. The obtained solid was filtered through acetonitrile, and magnesium sulfate and acid clay were added, followed by stirring for 30 minutes. After filtering the solution, it was recrystallized with acetonitrile/toluene and washed with toluene. The obtained solid was recrystallized again using acetonitrile/tert-butylmethylether and purified by sublimation, obtaining 1.0 g of compound 102 (yield 20%, MS[M+H]=569).

### Preparation Example 5. Synthesis of Compound B7

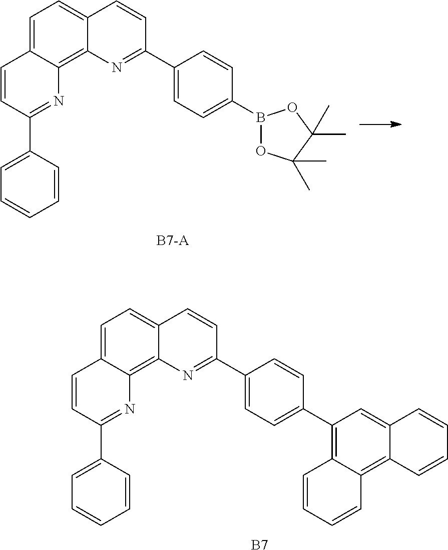 [SMILES: CC1(C)OB(c2ccc(-c3ccc4ccc5ccc(-c6ccccc6)nc5c4n3)cc2)OC1(C)C ; c1ccc(-c2ccc3ccc4ccc(-c5ccc(-c6cc7ccccc7c7ccccc67)cc5)nc4c3n2)cc1 ; "B7"]

19.5 g (42.7 mmol) of B7-A, 200 ml of toluene, 60 ml of ethanol, 42.7 mmol of 9-bromo-phenanthrene, 4 M potassium carbonate solution (30 ml), and 4.3 mmol of tetrakistriphenylphosphine palladium were mixed, heated and refluxed and stirred for 12 hours. After reducing the temperature to room temperature and adding 100 ml of water to terminate the reaction, the mixture was recrystallized using dichloromethane and filtered, obtaining 13 g of compound B7 (yield: 60%, MS[M+H]+=509).

### [Manufacturing Evaluation of Organic Light Emitting Element]

A hole injection layer (HIL, 80 Å, NPD+HATCN (10 wt %), a first hole transport layer (HTL1, 950 Å, NPD), a first light emitting layer (EML1, 250 Å, host (9, 10-di(naphtha-2-yl)anthracene)+dopant (1,6-bis(diphenylamine)pyrene, 3 wt %), a first electron transport layer (ETL1, 150 Å, 1,3,5-tri(m-pyridin-3-ylphenyl)benzene (TmPyPB), an n-type charge generation layer (n-CGL, 200 Å, nCGL compound shown in Table 1 below+Li (2 wt %), a p-type charge generation layer (p-CGL, 150 Å, NPD doped with 20 wt % of pCGL compound shown in Table 1 below), a second hole transport layer (HTL2, 300 Å, NPD), a second light emitting layer (EML2, 300 Å, host (CBP)+dopant (Ir(ppy)₃, 8 wt %), a second electron transport layer (ETL2, 200 Å, 2,2′,2″-(1,3,5-benzinetriyl)-tris(1-phenyl-1H-benzimidazole) (TPBi)), an electron injection layer (EIL, LIF, 10 Å) and a cathode (AI, 1500 Å) were sequentially stacked on an ITO (anode), forming an organic light emitting diode.

TABLE 1

| | | | | | Efficiency | T95 |
|---|---|---|---|---|---|---|
| | pCGL | nCGL | nCGL | | (relative | (relative |
| | compound | compounds(1) | compounds(2) | Voltage (V) | value, %) | value, %) |
| comparative | HATCN | Bphen | — | 9.9 | 100 | 100 |
| example 1 | | | | | | |
| comparative | HATCN | Bphen | C2 | 9.5 | 101 | 102 |
| example 2 | | | | | | |
| comparative | HATCN | B11 | Bphen | 9.6 | 100 | 100 |
| example 3 | | | | | | |
| comparative | HATCN | B11 | C2 | 9.4 | 102 | 101 |
| example 4 | | | | | | |
| comparative | PD1 | Bphen | — | 8.4 | 105 | 108 |
| example 5 | | | | | | |
| comparative | PD1 | Bphen | C2 | 8.2 | 110 | 115 |
| example 6 | | | | | | |
| comparative | PD1 | B11 | Bphen | 8.3 | 109 | 116 |
| example 7 | | | | | | |
| comparative | PD1 | B11 | C2 | 8.2 | 112 | 120 |
| example 8 | | | | | | |
| comparative | PD1 | B28 | C10 | 8.1 | 114 | 122 |
| example 9 | | | | | | |
| comparative | PD2 | Bphen | — | 8.3 | 108 | 110 |
| example 10 | | | | | | |
| comparative | PD2 | Bphen | C2 | 8.2 | 111 | 114 |
| example 11 | | | | | | |
| comparative | PD2 | B11 | Bphen | 8.2 | 113 | 116 |
| example 12 | | | | | | |
| comparative | PD2 | B11 | C2 | 8.1 | 115 | 122 |
| example 13 | | | | | | |
| comparative | PD2 | B28 | C10 | 8.1 | 115 | 121 |
| example 14 | | | | | | |
| comparative | PD3 | B11 | C2 | 8.0 | 116 | 124 |
| example 15 | | | | | | |
| comparative | PD3 | B28 | C10 | 7.9 | 118 | 125 |
| example 16 | | | | | | |
| comparative | PD4 | B11 | C2 | 8.0 | 115 | 125 |
| example 17 | | | | | | |
| comparative | PD4 | B28 | C10 | 8.0 | 117 | 123 |
| example 18 | | | | | | |

TABLE 2

| | | | | | Efficiency | T95 |
|---|---|---|---|---|---|---|
| | pCGL | nCGL | nCGL | | (relative | (relative |
| | compound | compounds(1) | compounds(2) | Voltage (V) | value, %) | value, %) |
| embodiment 1 | 1 | Bphen | — | 8.1 | 114 | 128 |
| embodiment 2 | 1 | Bphen | C2 | 7.8 | 122 | 136 |
| embodiment 3 | 1 | B11 | Bphen | 7.9 | 120 | 141 |
| embodiment 4 | 1 | B11 | C2 | 7.8 | 123 | 149 |
| embodiment 5 | 1 | B11 | — | 7.9 | 121 | 150 |
| embodiment 6 | 1 | — | C2 | 7.8 | 122 | 147 |
| embodiment 7 | 1 | B28 | C10 | 7.7 | 125 | 154 |
| embodiment 8 | 26 | B11 | — | 7.7 | 126 | 150 |
| embodiment 9 | 26 | — | C2 | 7.6 | 128 | 148 |
| embodiment 10 | 26 | B11 | C2 | 7.7 | 126 | 155 |
| embodiment 11 | 26 | B28 | C10 | 7.6 | 129 | 157 |
| embodiment 12 | 26 | B7 | C19 | 7.6 | 131 | 160 |
| embodiment 13 | 52 | B11 | — | 7.7 | 126 | 152 |
| embodiment 14 | 52 | — | C2 | 7.7 | 125 | 151 |
| embodiment 15 | 52 | B11 | C2 | 7.7 | 126 | 153 |
| embodiment 16 | 52 | B28 | C10 | 7.7 | 127 | 155 |
| embodiment 17 | 52 | B7 | C19 | 7.6 | 130 | 156 |
| embodiment 18 | 102 | B11 | — | 7.8 | 126 | 150 |
| embodiment 19 | 102 | — | C2 | 7.7 | 125 | 152 |
| embodiment 20 | 102 | B11 | C2 | 7.7 | 128 | 154 |
| embodiment 21 | 102 | B28 | C10 | 7.7 | 129 | 155 |
| embodiment 22 | 102 | B7 | C19 | 7.6 | 132 | 158 |

In Tables 1 and 2 above, HATCN is 1,4,5,8,9,11-hexaazatriphenylenehexacarbonitile, Bphen is bathophenanthroline, and PD1 to PD4 are as follows.

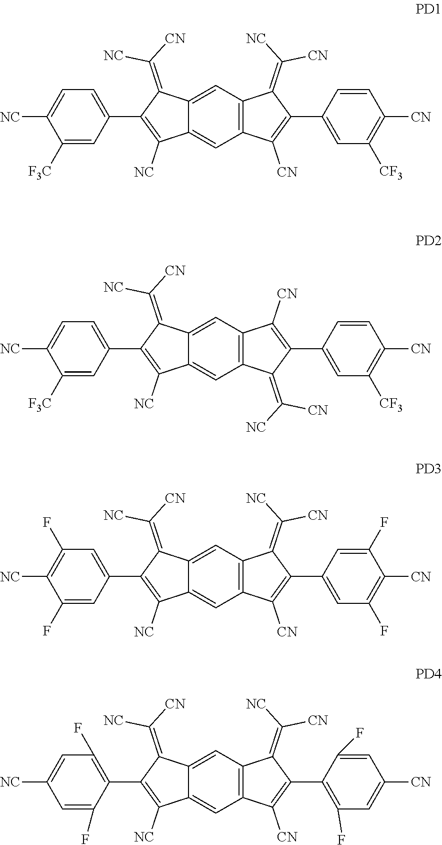 [SMILES: N#CC(C#N)=C1C(c2ccc(C#N)c(C(F)(F)F)c2)=C(C#N)c2cc3c(cc21)C(=C(C#N)C#N)C(c1ccc(C#N)c(C(F)(F)F)c1)=C3C#N ; N#CC(C#N)=C1C(c2ccc(C#N)c(C(F)(F)F)c2)=C(C#N)c2cc3c(cc21)C(C#N)=C(c1ccc(C#N)c(C(F)(F)F)c1)C3=C(C#N)C#N ; N#CC(C#N)=C1C(c2cc(F)c(C#N)c(F)c2)=C(C#N)c2cc3c(cc21)C(=C(C#N)C#N)C(c1cc(F)c(C#N)c(F)c1)=C3C#N ; N#CC(C#N)=C1C(c2c(F)cc(C#N)cc2F)=C(C#N)c2cc3c(cc21)C(=C(C#N)C#N)C(c1c(F)cc(C#N)cc1F)=C3C#N]

Referring to Tables 1 and 2, the organic light emitting element of the embodiment which uses the first compound of the disclosure in the p-type charge generation layer and the second compound in the n-type charge generation layer has better efficiency, longer lifespan, and lower driving voltage than the organic light emitting element of the comparative example.

Referring to Table 2, the embodiments (embodiments 2-4, 7, 10-12, 15-17, and 20-22) in which the n-type charge generation layer simultaneously includes one type of third compound and one type of fourth compound have more excellent efficiency, a longer lifespan, or a lower driving voltage than the embodiments (embodiments 1, 5, 6, 8, 9, 13, 14, 18, and 19) in which only one type of second compound is included.

More specifically, comparison between embodiment 1 and embodiment 2 reveals that embodiment 2 in which the n-type charge generation layer includes Bphene which is the third compound and C2 which is the fourth compound has more excellent efficiency, a longer lifespan, and lower driving voltage than embodiment 1 in which only Bphen is included. It may be identified that this tendency is also shown by comparing embodiment 4 and embodiment 5, and similar tendencies are observed in other embodiments.

The above description has been presented to enable any person skilled in the art to make and use the technical idea of the disclosure, and has been provided in the context of a particular application and its requirements. Various modifications, additions and substitutions to the described embodiments will be readily apparent to those skilled in the art, and the general principles defined herein may be applied to other embodiments and applications. The above description and the accompanying drawings provide an example of the technical idea of the disclosure for illustrative purposes only. That is, the disclosed embodiments are intended to illustrate the scope of the technical idea of the disclosure.

What is claimed:

1. An organic light emitting element, comprising:
a first electrode;
a second electrode; and
an organic material layer positioned between the first electrode and the second electrode, wherein the organic material layer includes a first compound represented by chemical formula 1 below and a second compound represented by chemical formula 2 below, 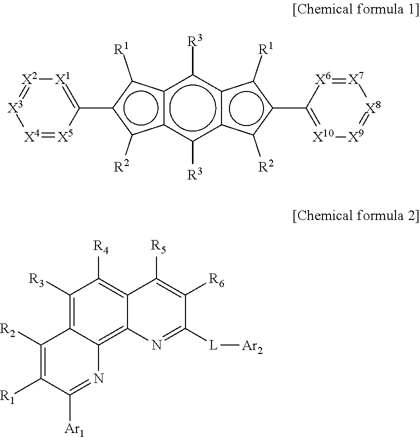
in chemical formula 1,
each R¹ and each R² are each independently selected from the group consisting of hydrogen; deuterium; tritium; halogen; a cyano group; an alkyl group of C₁-C₅₀; a haloalkyl group of C₁-C₅₀; an alkoxy group of C₁-C₃₀; a haloalkoxy group of C₁-C₃₀; an aryl group of C₆-C₆₀; a haloaryl group of C₆-C₆₀; a heterocyclic group of C₂-C₆₀ including at least one heteroatom among O, N, S, Si and P; a haloheterocyclic group of C₂-C₆₀ including at least one heteroatom among O, N, S, Si and P; and malononitrile,
wherein each R³ is independently selected from the group consisting of hydrogen; deuterium; tritium; halogen; a cyano group; a malononitrile group; an alkyl group of C₁-C₅₀; a haloalkyl group of C₁-C₅₀; an alkoxy group of C₁-C₃₀; a haloalkoxy group of C₁-C₃₀; an aryl group of C₆-C₆₀; a haloaryl group of C₆-C₆₀; a heterocyclic group of C₂-C₆₀ including at least one heteroatom among O, N, S, Si and P; and a haloheterocyclic group of C₂-C₆₀ including at least one heteroatom among O, N, S, Si and P,
wherein X¹ to X⁵ are each independently CR^{a} or N, and at least two of X¹ to X⁵ are CR^{a}, wherein each R^{a} is independently selected from among hydrogen; deuterium; tritium; halogen, a cyano group; an alkyl group of C₁-C₅₀; and an alkoxy group of C₁-C₅₀, wherein at least one R^{a} is a halogen or a cyano group,
wherein X⁶ to X¹⁰ are each independently CR^{b} or N, and at least two of X⁶ to X¹⁰ are CR^{b}, wherein each R^{b} is independently selected from among hydrogen; deuterium; tritium; halogen, a cyano group; an alkyl group of C₁-C₅₀; and an alkoxy group of C₁-C₅₀, wherein at least one R^{b} is a halogen or a cyano group,
wherein in chemical formula 1, the alkyl group, the haloalkyl group, the alkoxy group, the haloalkoxy group, the aryl group, the haloaryl group, the heterocyclic group, and the haloheterocyclic group each may be additionally substituted with one or more substituents selected from the group consisting of deuterium; a nitro group; a cyano group; an amino group; an alkoxy group of C₁-C₂₀; a haloalkoxy group of C₁-C₂₀; an alkyl group of C₁-C₂₀; a haloalkyl group of C₁-C₂₀; an alkenyl group of C₂-C₂₀; an alkynyl group of C₂-C₂₀; an aryl group of C₆-C₂₀; an aryl group of C₆-C₂₀ substituted with deuterium; a fluorenyl group; a heterocyclic group of C₂-C₂₀; an alkylsilyl group of C₃-C₆₀; an arylsilyl group of C₁₈-C₆₀; and an alkylarylsilyl group of C₈-C₆₀,
wherein in chemical formula 2,
R¹ to R⁶ are each independently selected from the group consisting of hydrogen; deuterium; tritium; halogen; cyano group; nitro group; aryl group of C₆-C₆₀; a fluorenyl group; a heterocyclic group of C₂-C₆₀ containing at least one heteroatom of O, N, S, Si, and P; a fused ring group of an aliphatic ring of C₃-C₆₀ and an aromatic ring of C₆-C₆₀; an alkyl group of C₁-C₅₀; an alkenyl group of C₂-C₂₀; an alkynyl group of C₂-C₂₀; an alkoxy group of C₁-C₃₀; an aryloxy group of C₆-C₃₀; an alkylsilyl group of C₃-C₆₀; an arylsilyl group of C₁₈-C₆₀; and an alkylarylsilyl group of C₈-C₆₀,
wherein Ar1 and Ar2 are each independently selected from the group consisting of hydrogen; deuterium; tritium; halogen; cyano group; nitro group; aryl group of C₆-C₆₀; a fluorenyl group; a heterocyclic group of C₂-C₆₀ containing at least one heteroatom of O, N, S, Si, and P; a fused ring group of an aliphatic ring of C₃-C₆₀ and an aromatic ring of C₆-C₆₀; an alkyl group of C₁-C₅₀; an alkenyl group of C₂-C₂₀; an alkynyl group of C₂-C₂₀; an alkoxy group of C₁-C₃₀; an aryloxy group of C₆-C₃₀; an alkylsilyl group of C₃-C₆₀; an arylsilyl group of C₁₈-C₆₀; and an alkylarylsilyl group of C₈-C₆₀, wherein L is selected from the group consisting of a divalent heterocyclic group of C₂-C₆₀ containing an arylene group of C₆-C₆₀; a fluorenyl group; a divalent heterocyclic group of C₂-C₆₀ containing at least one heteroatom of O, N, S, Si, and P; and a divalent fused ring group of an aliphatic ring of C₃-C₆₀ and an aromatic ring of C₆-C₆₀, and
wherein in chemical formula 2, the alkyl group, the fluorenyl group, the heterocyclic group, the fused ring group, the arylene group, the fluorenyl group, the divalent heterocyclic group, and the divalent fused ring group each may be additionally substituted with one or more substituents selected from the group consisting of deuterium; a nitro group; a cyano group; a halogen group; an amino group; an alkoxyl group of C₁-C₂₀; an alkyl group of C₁-C₂₀; an alkenyl group of C₂-C₂₀; an alkynyl group of C₂-C₂₀; an aryl group of C₆-C₂₀; an aryl group of C₆-C₂₀ substituted with deuterium; a fluorenyl group; a heterocyclic group of C₂-C₂₀; an alkylsilyl group of C₃-C₆₀; an arylsilyl group of C₁₈-C₆₀; and an alkylarylsilyl group of C₈-C₆₀.

2. The organic light emitting element of claim 1, wherein the second compound includes a third compound which is represented by chemical formula 2 and in which Ar₂ is not phenanthroline; and a fourth compound which is represented by chemical formula 2 and in which Ar₂ is phenanthroline.

3. The organic light emitting element of claim 1, wherein the first compound is represented by either chemical formula 3 or chemical formula 4 below: 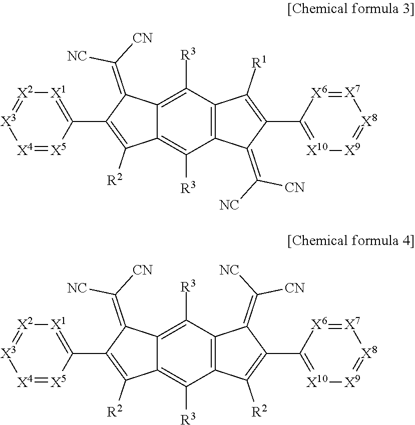
in chemical formula 3 and chemical formula 4,
R¹ to R³ and X¹ to X¹⁰ are the same as R¹ to R³ and X¹ to X¹⁰ defined in chemical formula 1.

4. The organic light emitting element of claim 1, wherein the first compound is represented by either chemical formula 5 or chemical formula 6 below: 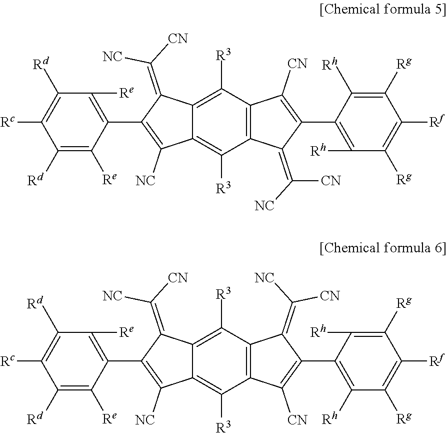
in chemical formula 5 and chemical formula 6,
R^{c} and R^{d} are each independently selected from among hydrogen; deuterium; tritium; halogen; a cyano group; an alkyl group of C₁-C₅₀; and an alkoxy group of C₁-C₅₀, wherein each R^{e} is independently selected from the group consisting of hydrogen; deuterium; tritium; halogen; and a cyano group, wherein R^{f} and R⁹ are each independently selected from among hydrogen; deuterium; tritium; halogen; a cyano group; an alkyl group of C₁-C₅₀; and an alkoxy group of C₁-C₅₀, wherein each R^{h} is independently selected from the group consisting of hydrogen; deuterium; tritium; halogen; and a cyano group, and wherein R³ is the same as R³ defined in chemical formula 1.

5. The organic light emitting element of claim 4, wherein in chemical formula 5,
R^{c} is a halogen; or cyano group, wherein i) one R^{d} is hydrogen; deuterium; or tritium; and the other R^{d} is halogen; or a cyano group; or ii) two R^{d}s are each independently a halogen; or cyano group, wherein one R^{e} is hydrogen; deuterium; or tritium, and the other R^{e} is a halogen or cyano group, and wherein R^{f} is a halogen; or cyano group, wherein i) one R^{g} is hydrogen; deuterium; or tritium; and the other R^{g} is halogen; or a cyano group; or ii) two R^{g}s are each independently a halogen; or cyano group, wherein one R^{h} is hydrogen; deuterium; or tritium, and the other R^{h} is a halogen or cyano group, wherein in chemical formula 6,
R^{c} is a halogen; or cyano group, wherein i) one R^{d} is hydrogen; deuterium; or tritium; and the other R^{d} is halogen; or a cyano group; or ii) two R^{d}s are each independently a halogen; or cyano group, wherein one R^{e} is hydrogen; deuterium; or tritium, and the other R^{e} is a halogen or cyano group, and wherein R^{f} is a halogen; or cyano group, wherein i) one R^{g} is hydrogen; deuterium; or tritium; and the other R^{g} is halogen; or a cyano group; or ii) two R^{g}s are each independently a halogen; or cyano group, and wherein one R^{h} is hydrogen; deuterium; or tritium, and the other R^{h} is a halogen or cyano group.

6. The organic light emitting element of claim 1, wherein the first compound is represented by any one of chemical formula 7 to chemical formula 16 below: 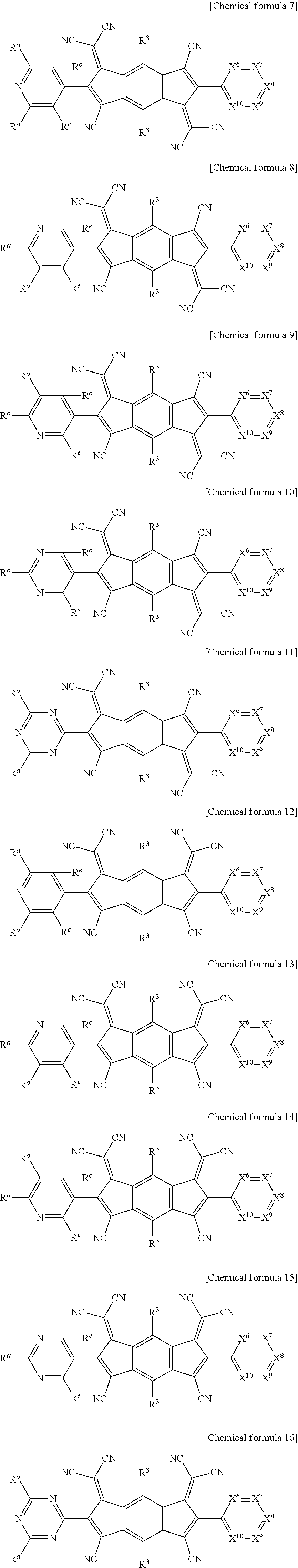
in chemical formula 7 to chemical formula 16,
R^{a}, R³ and X⁶ to X¹⁰ are the same as R^{a}, R³ and X⁶ to X¹⁰ defined in chemical formula 1, and wherein each R^{e} is independently selected from the group consisting of hydrogen; deuterium; tritium; halogen; and a cyano group.

7. The organic light emitting element of claim 6, wherein in chemical formula 7 to chemical formula 16,
R^{a}, R³ and X⁶ to X¹⁰ are the same as R^{a}, R³ and X⁶ to X¹⁰ defined in chemical formula 1,
wherein in chemical formula 7, one R^{e} is hydrogen; deuterium; or tritium, and the other R^{e} is a halogen or cyano group,
wherein in chemical formula 8, one R^{e} is hydrogen; deuterium; or tritium, and the other R^{e} is a halogen or cyano group,
wherein in chemical formula 9, one R^{e} is hydrogen; deuterium; or tritium, and the other R^{e} is a halogen or cyano group,
wherein in chemical formula 10, one R^{e} is hydrogen; deuterium; or tritium, and the other R^{e} is a halogen or cyano group,
wherein in chemical formula 12, one R^{e} is hydrogen; deuterium; or tritium, and the other R^{e} is a halogen or cyano group,
wherein in chemical formula 13, one R^{e} is hydrogen; deuterium; or tritium, and the other R^{e} is a halogen or cyano group,
wherein in chemical formula 14, one R^{e} is hydrogen; deuterium; or tritium, and the other R^{e} is a halogen or cyano group, and
wherein in chemical formula 15, one R^{e} is hydrogen; deuterium; or tritium, and the other R^{e} is a halogen or cyano group.

8. The organic light emitting element of claim 1, wherein the first compound comprises one or more of compounds 1 to 150 below:

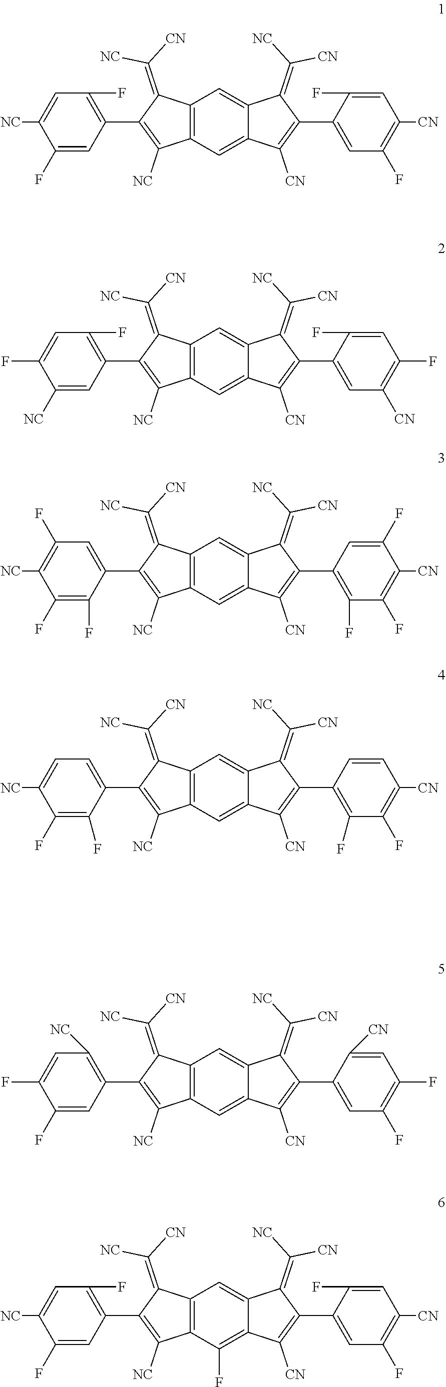

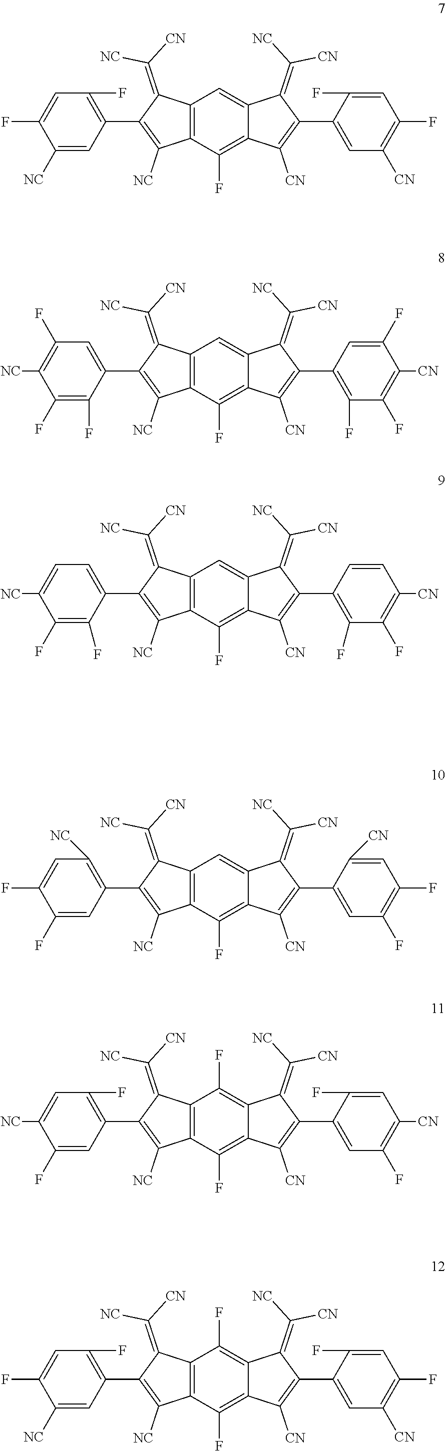

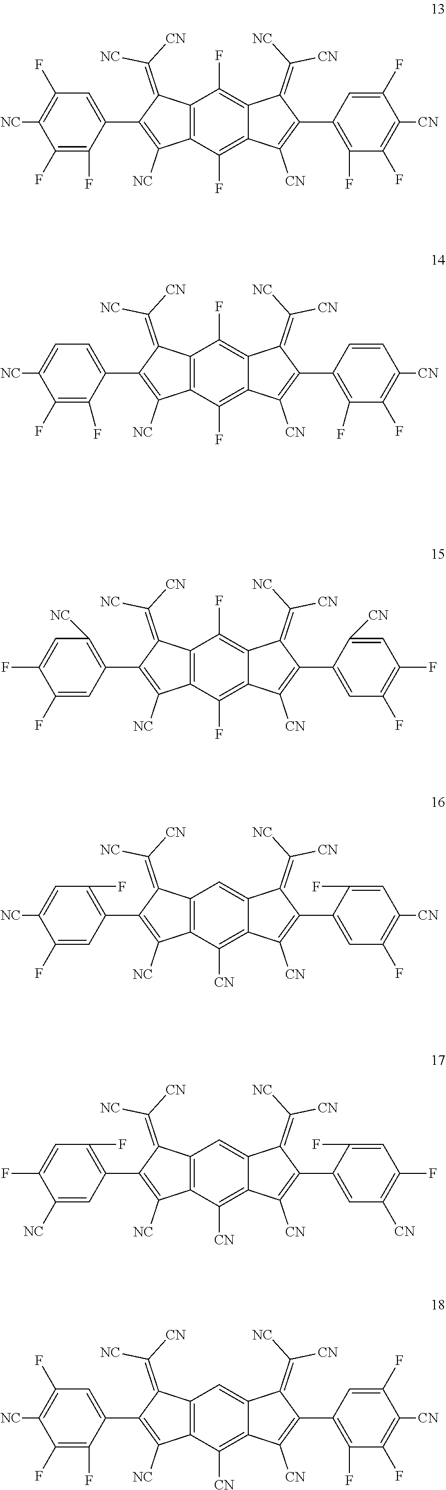

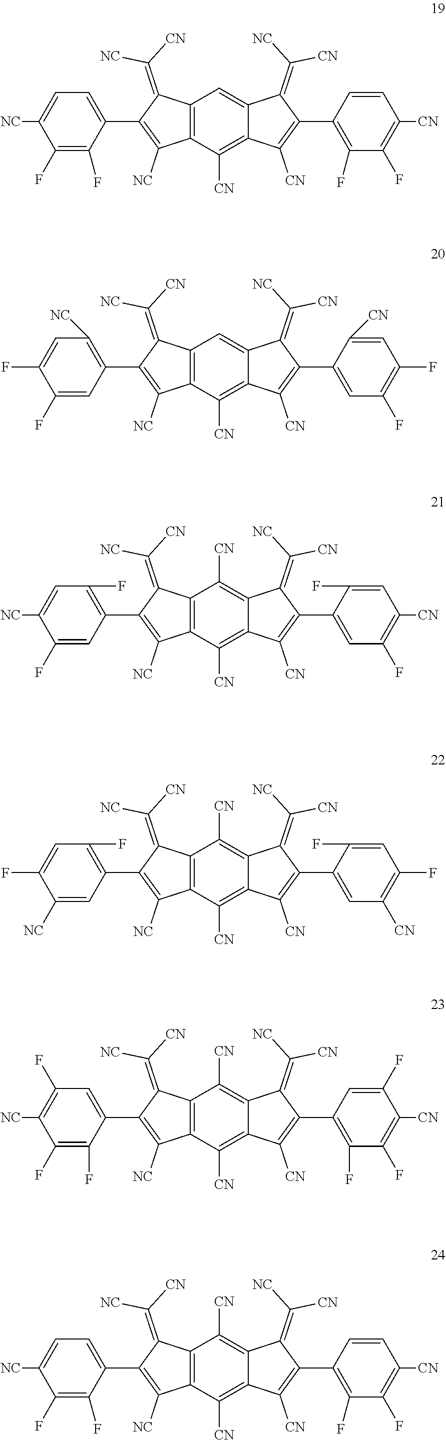

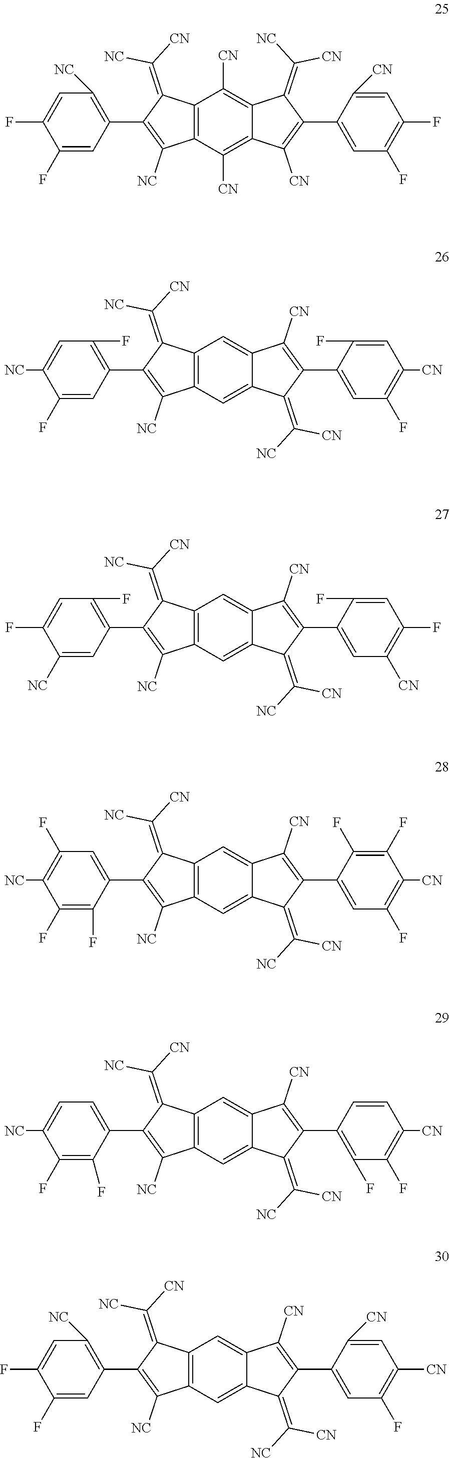

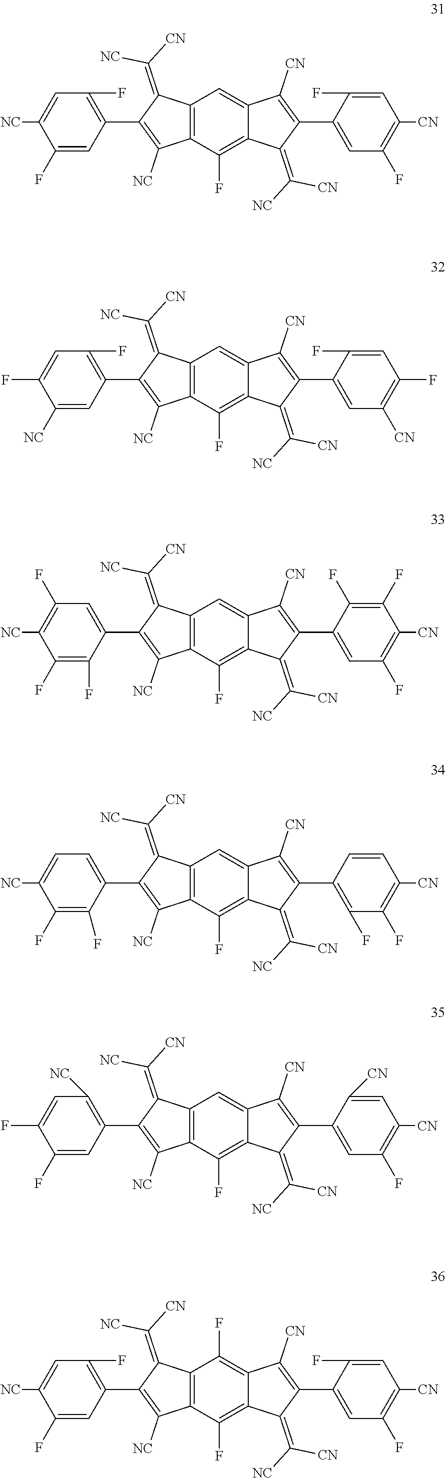

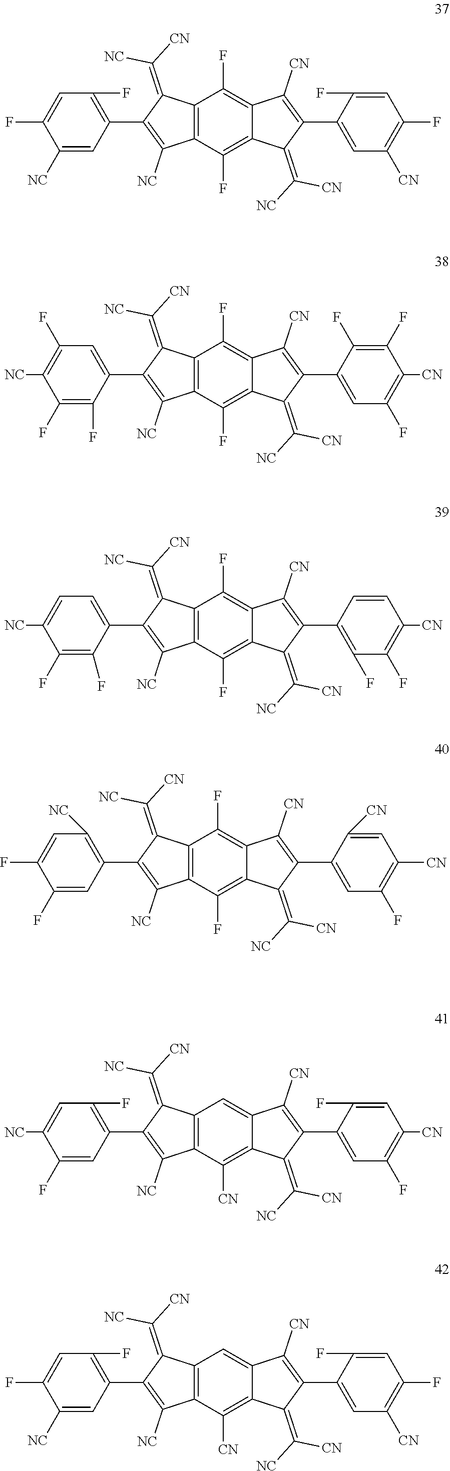

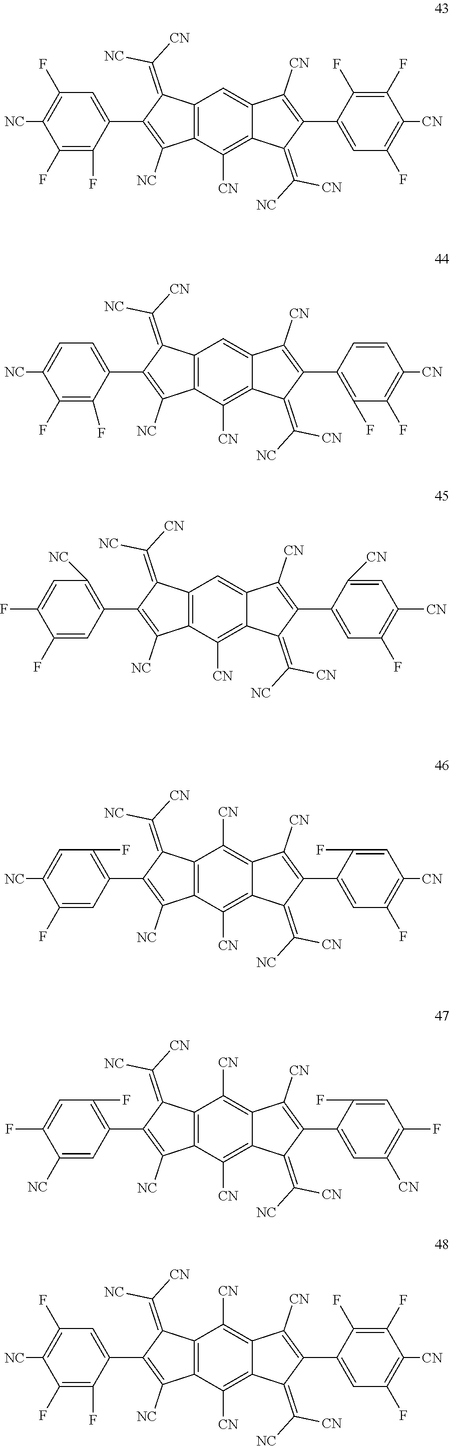

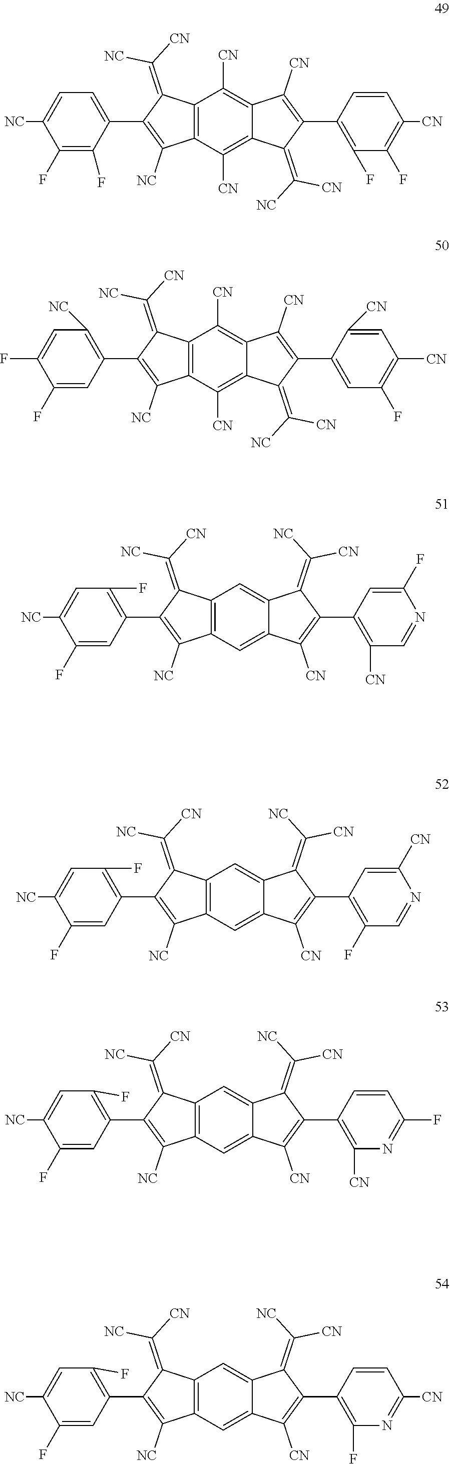

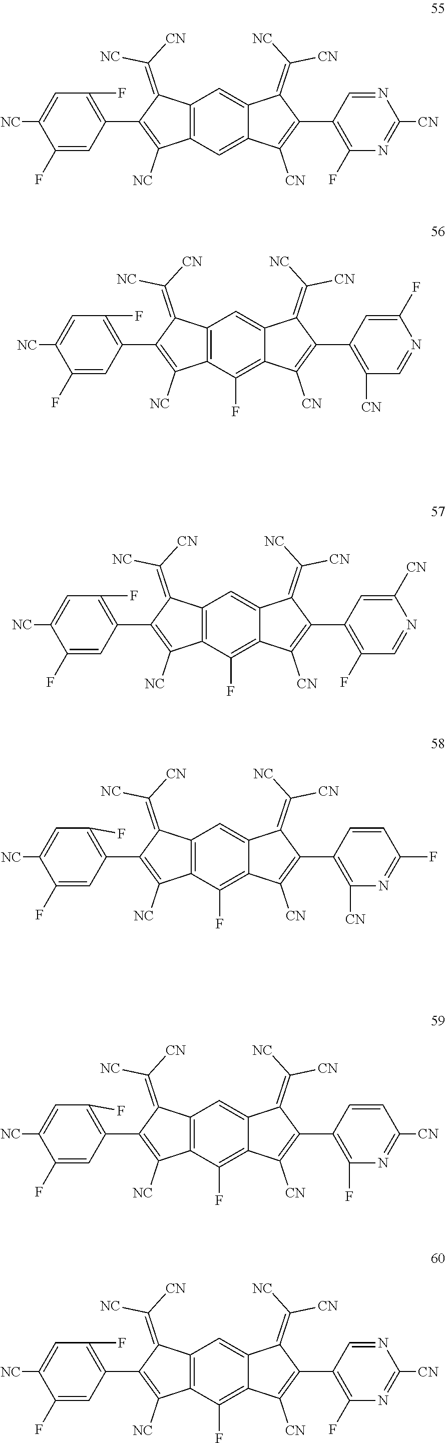

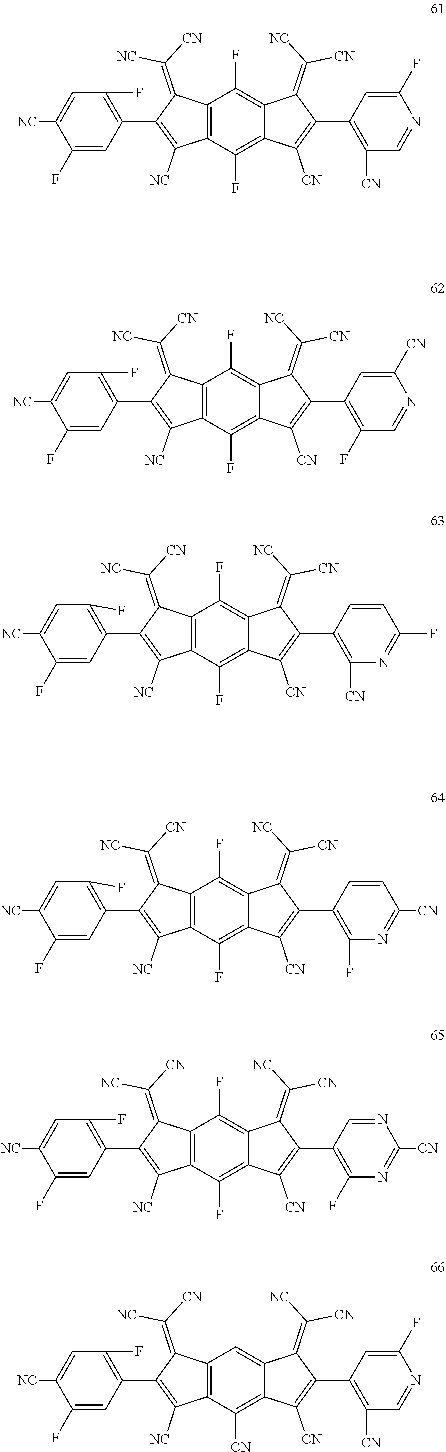

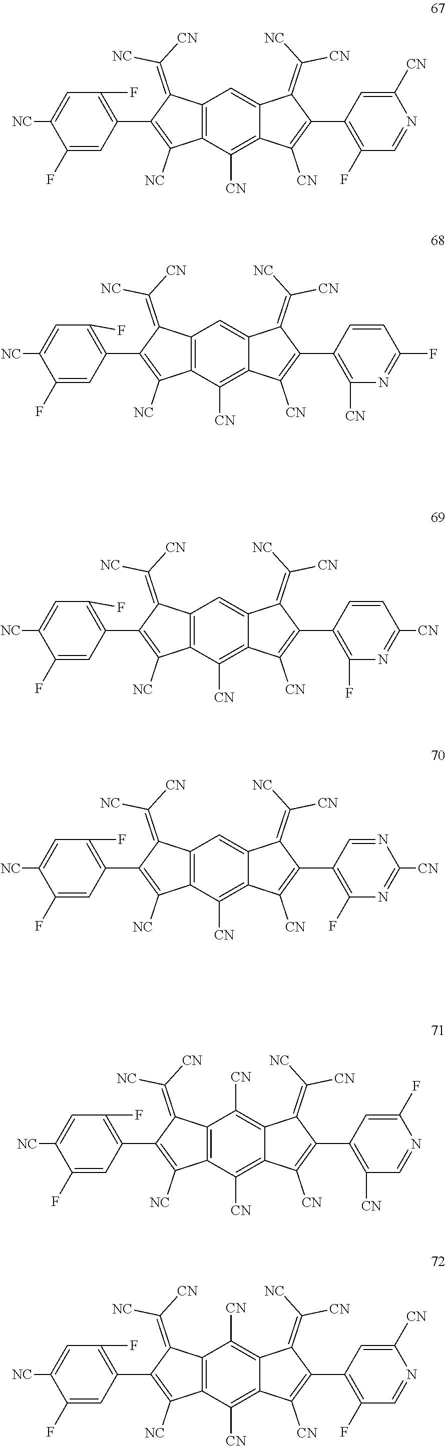

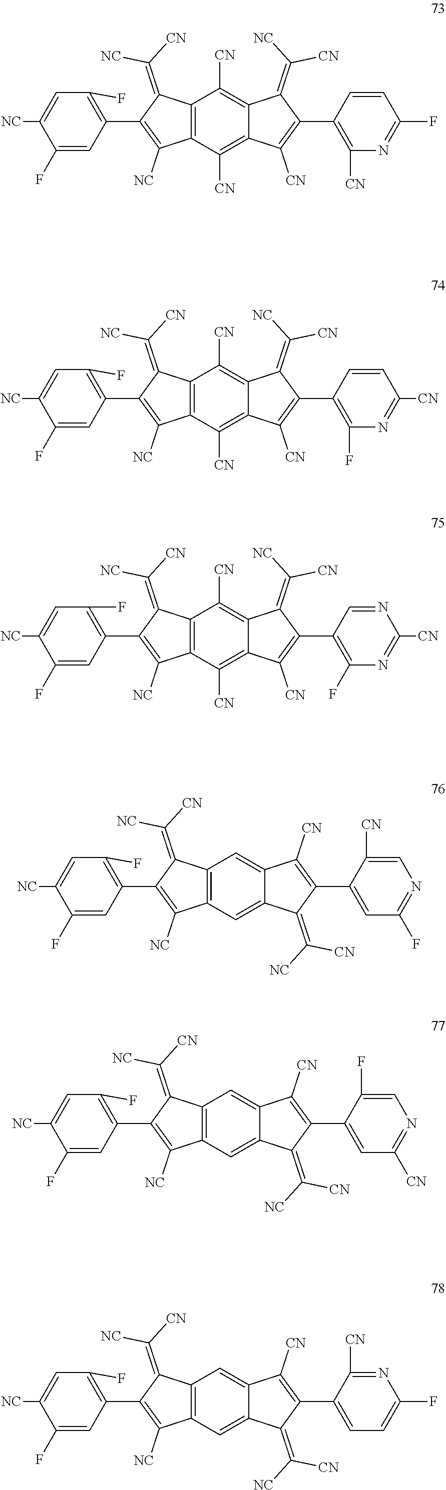

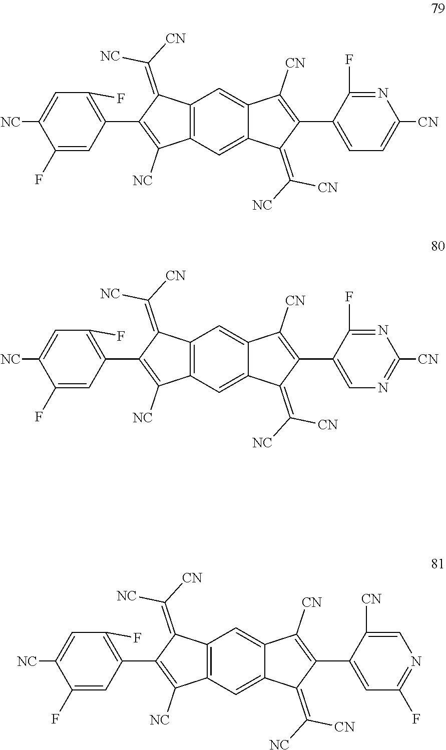

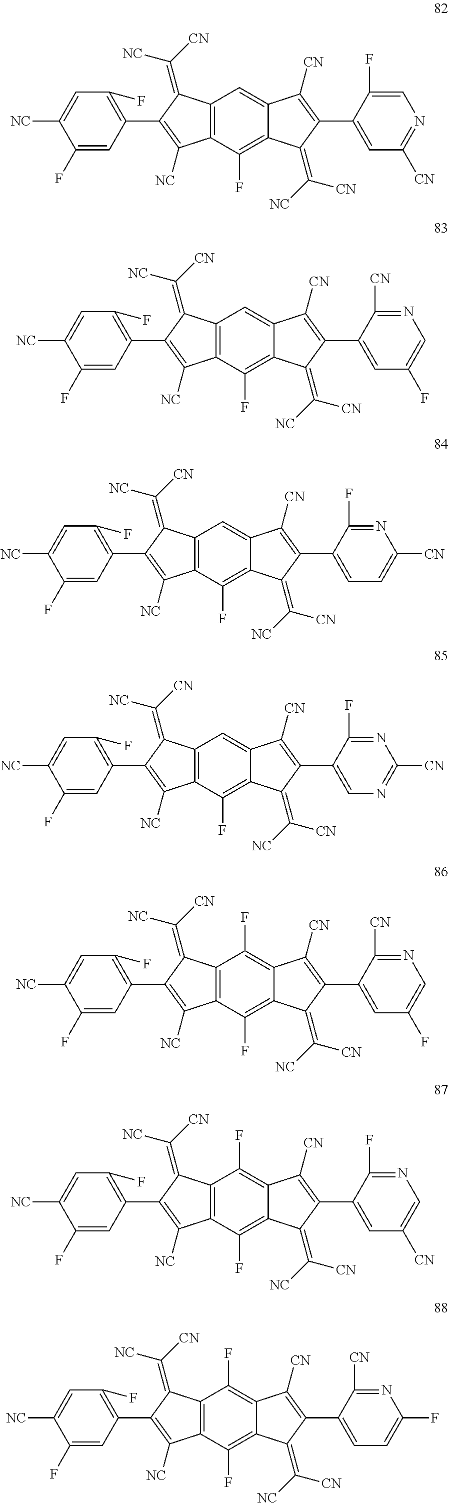

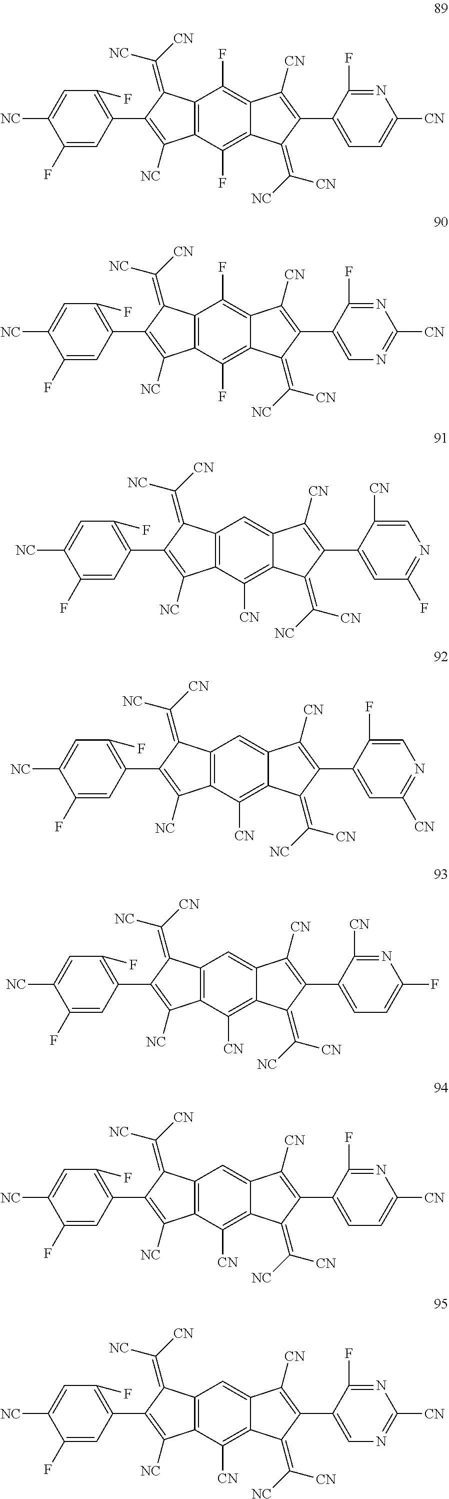

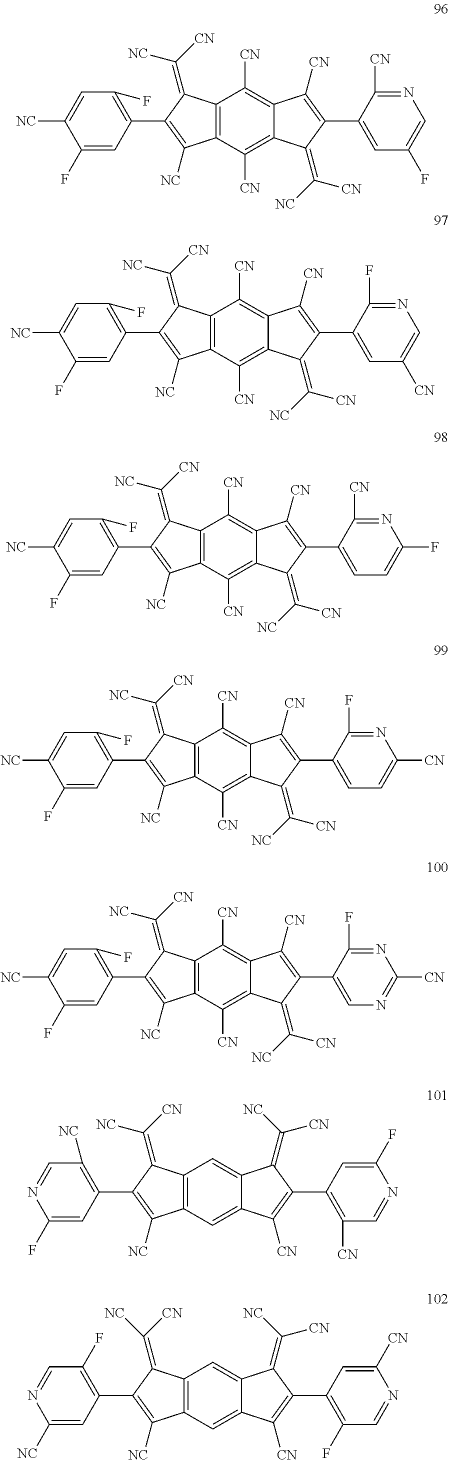

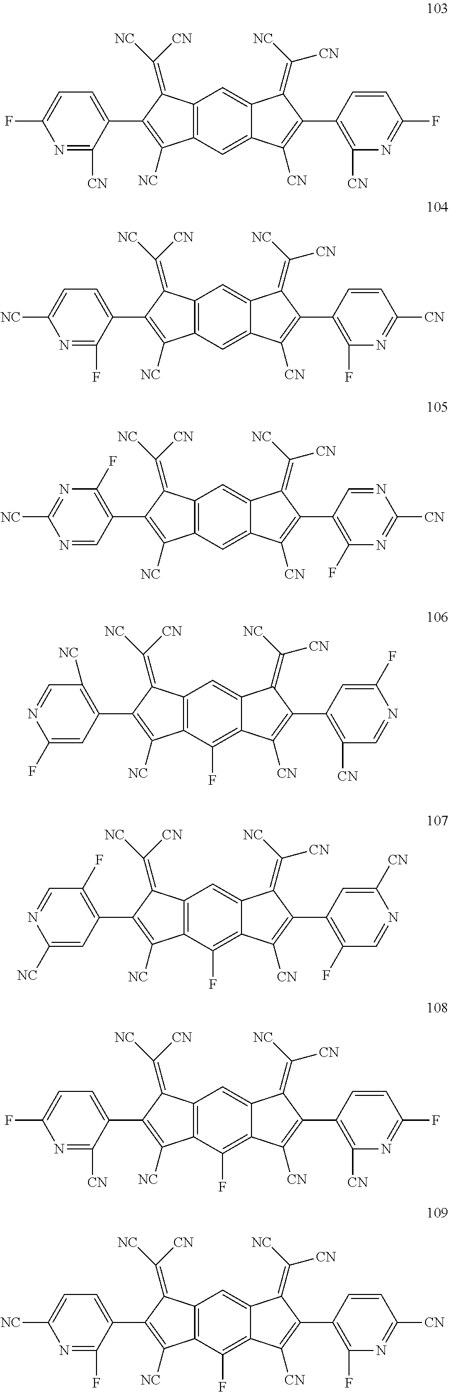

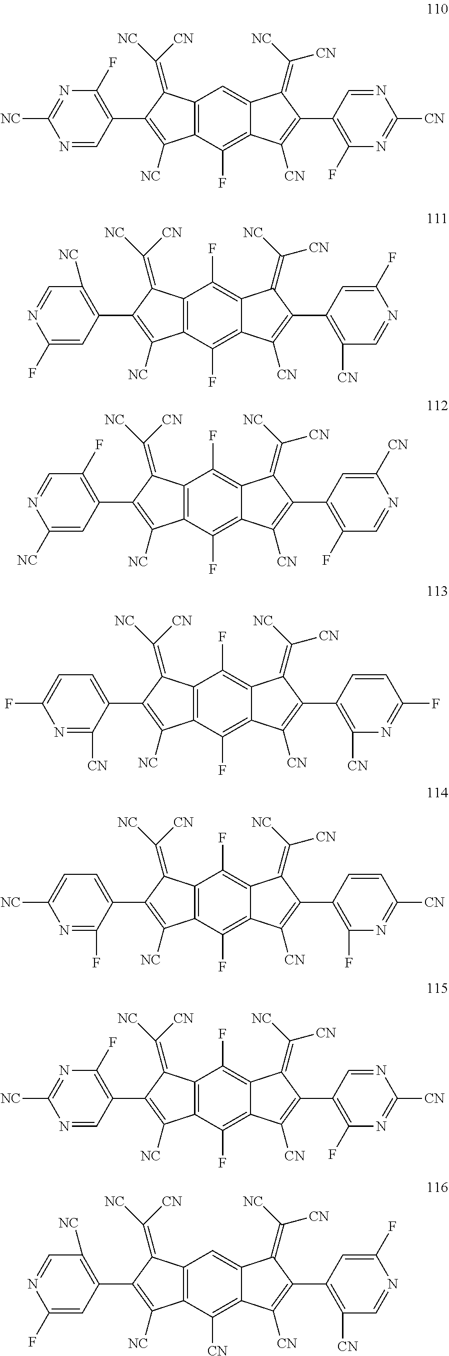

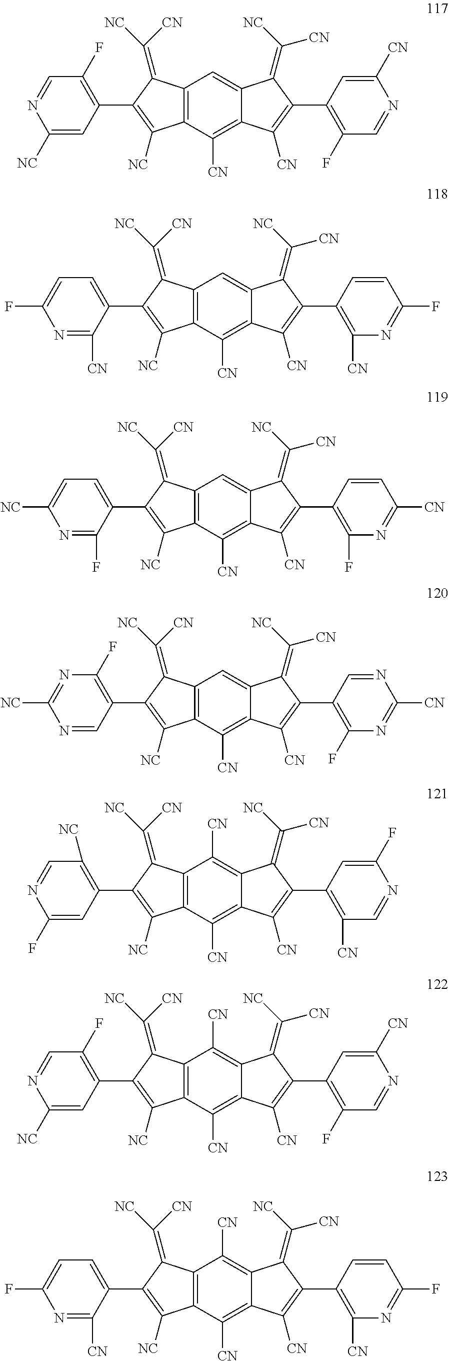

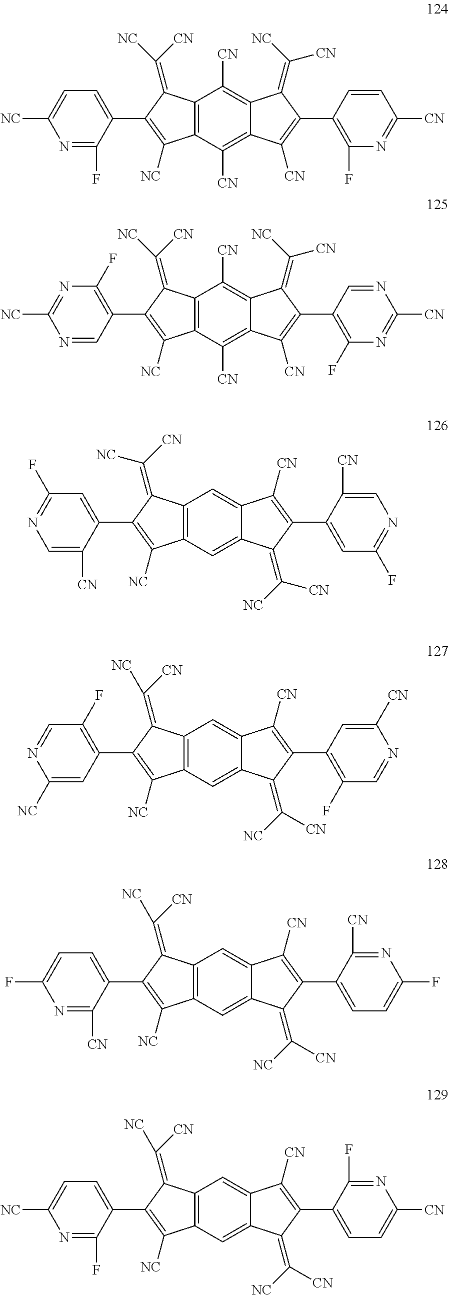

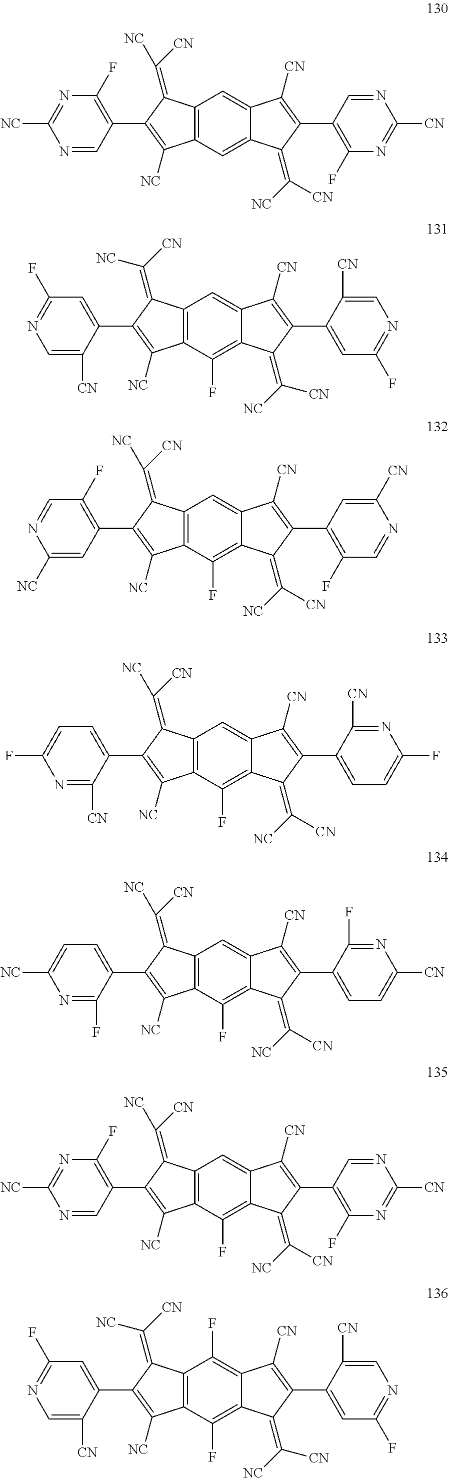

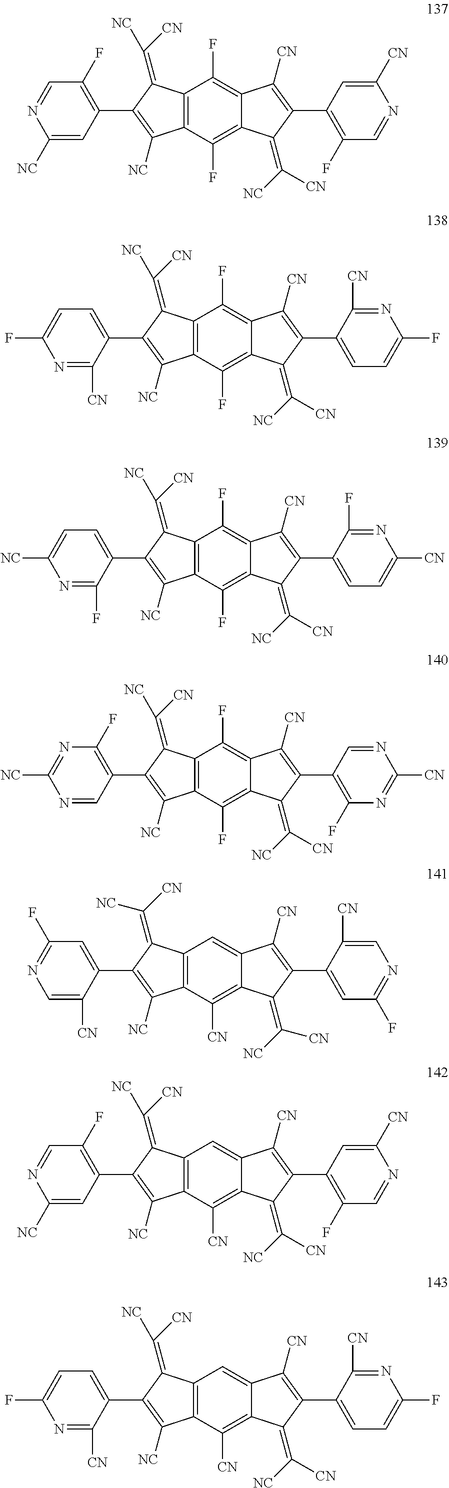

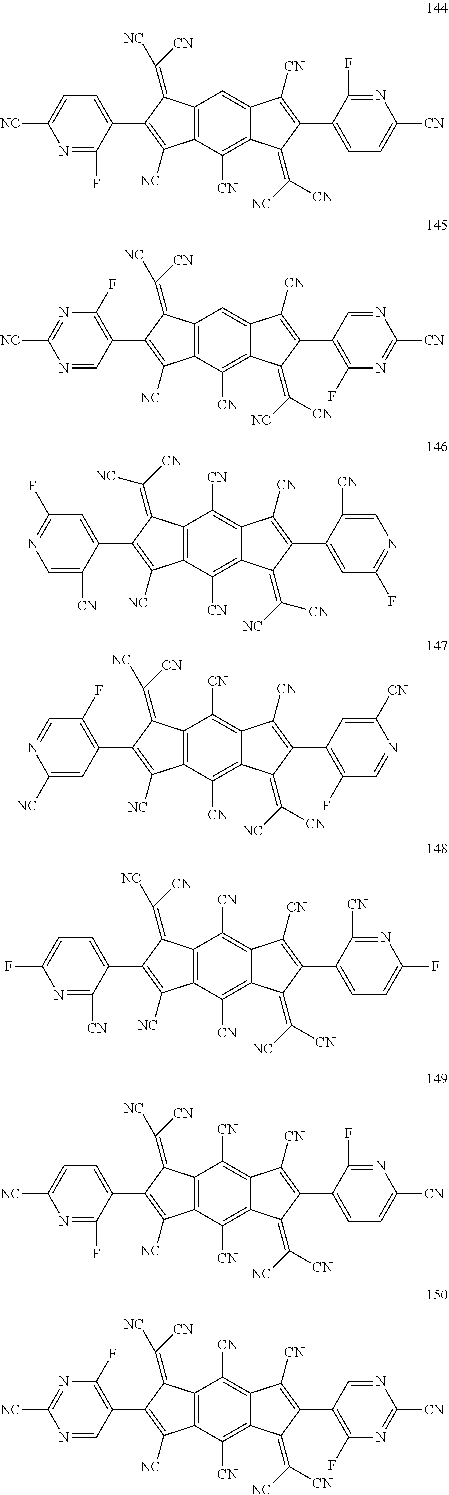

9. The organic light emitting element of claim 1, wherein the second compound comprises one or more of compounds B1 to B40 and C1 to C20 below:

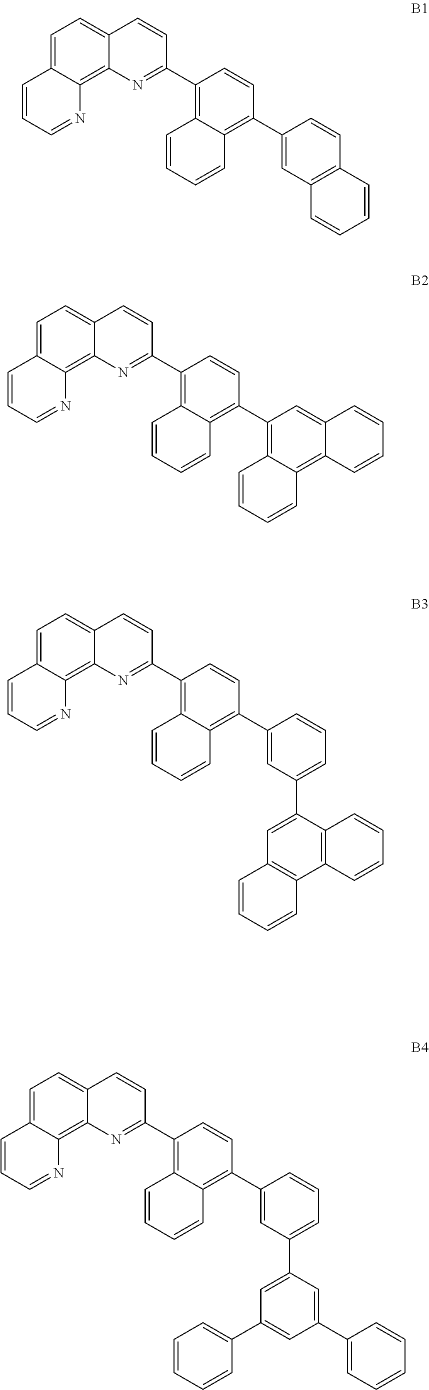 [SMILES: c1ccc2cc(-c3ccc(-c4ccc5ccc6cccnc6c5n4)c4ccccc34)ccc2c1 ; c1ccc2c(c1)cc(-c1ccc(-c3ccc4ccc5cccnc5c4n3)c3ccccc13)c1ccccc12 ; c1cc(-c2ccc(-c3ccc4ccc5cccnc5c4n3)c3ccccc23)cc(-c2cc3ccccc3c3ccccc23)c1 ; c1ccc(-c2cc(-c3ccccc3)cc(-c3cccc(-c4ccc(-c5ccc6ccc7cccnc7c6n5)c5ccccc45)c3)c2)cc1]

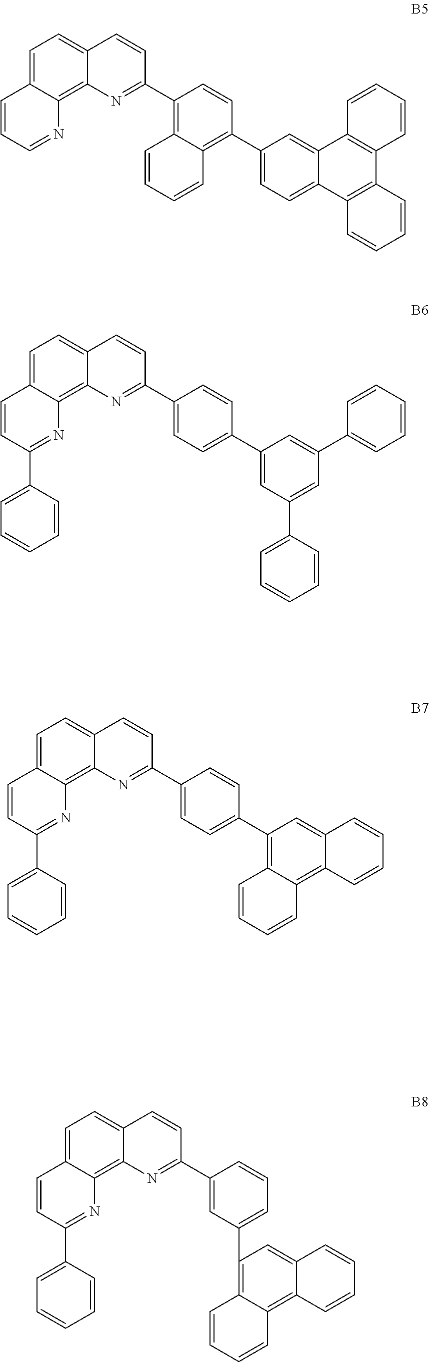 [SMILES: c1cnc2c(c1)ccc1ccc(-c3ccc(-c4ccc5c6ccccc6c6ccccc6c5c4)c4ccccc34)nc12 ; c1ccc(-c2cc(-c3ccccc3)cc(-c3ccc(-c4ccc5ccc6ccc(-c7ccccc7)nc6c5n4)cc3)c2)cc1 ; c1ccc(-c2ccc3ccc4ccc(-c5ccc(-c6cc7ccccc7c7ccccc67)cc5)nc4c3n2)cc1 ; c1ccc(-c2ccc3ccc4ccc(-c5cccc(-c6cc7ccccc7c7ccccc67)c5)nc4c3n2)cc1]

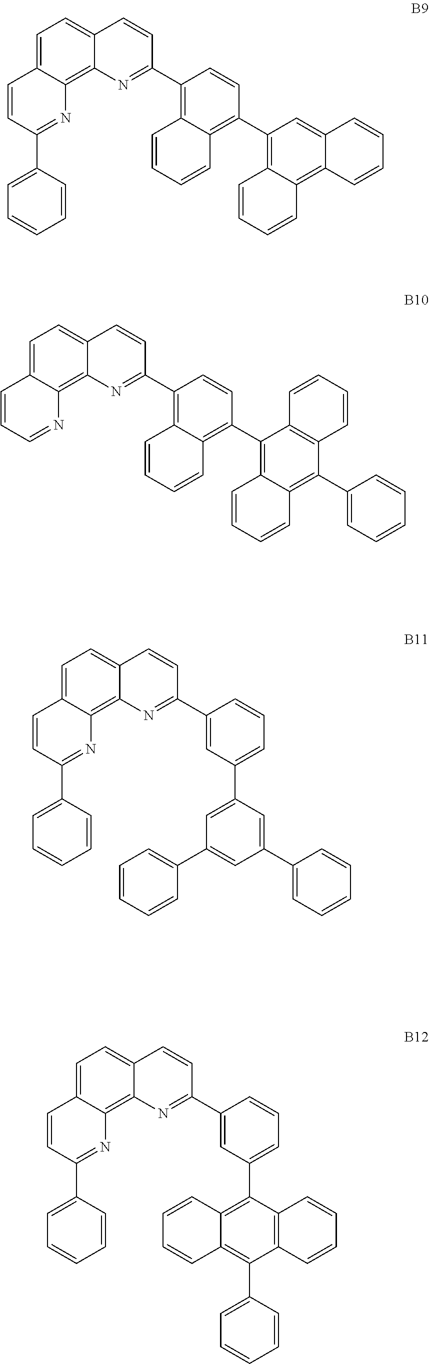 [SMILES: c1ccc(-c2ccc3ccc4ccc(-c5ccc(-c6cc7ccccc7c7ccccc67)c6ccccc56)nc4c3n2)cc1 ; c1ccc(-c2c3ccccc3c(-c3ccc(-c4ccc5ccc6cccnc6c5n4)c4ccccc34)c3ccccc23)cc1 ; c1ccc(-c2cc(-c3ccccc3)cc(-c3cccc(-c4ccc5ccc6ccc(-c7ccccc7)nc6c5n4)c3)c2)cc1 ; c1ccc(-c2ccc3ccc4ccc(-c5cccc(-c6c7ccccc7c(-c7ccccc7)c7ccccc67)c5)nc4c3n2)cc1]

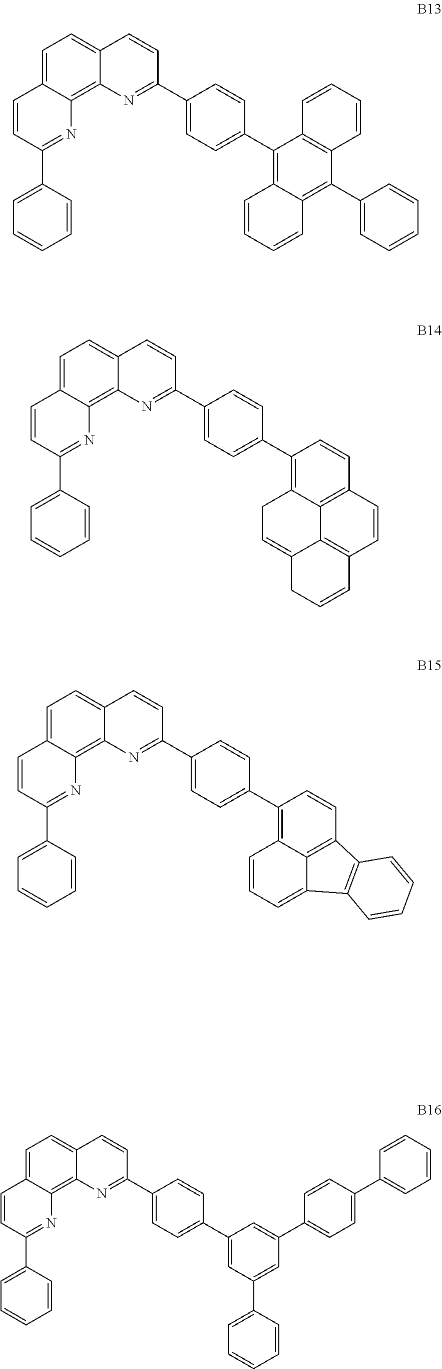 [SMILES: c1ccc(-c2ccc3ccc4ccc(-c5ccc(-c6c7ccccc7c(-c7ccccc7)c7ccccc67)cc5)nc4c3n2)cc1 ; C1=Cc2ccc3ccc(-c4ccc(-c5ccc6ccc7ccc(-c8ccccc8)nc7c6n5)cc4)c4c3c2C(=CC4)C1 ; c1ccc(-c2ccc3ccc4ccc(-c5ccc(-c6ccc7c8c(cccc68)-c6ccccc6-7)cc5)nc4c3n2)cc1 ; c1ccc(-c2ccc(-c3cc(-c4ccccc4)cc(-c4ccc(-c5ccc6ccc7ccc(-c8ccccc8)nc7c6n5)cc4)c3)cc2)cc1]

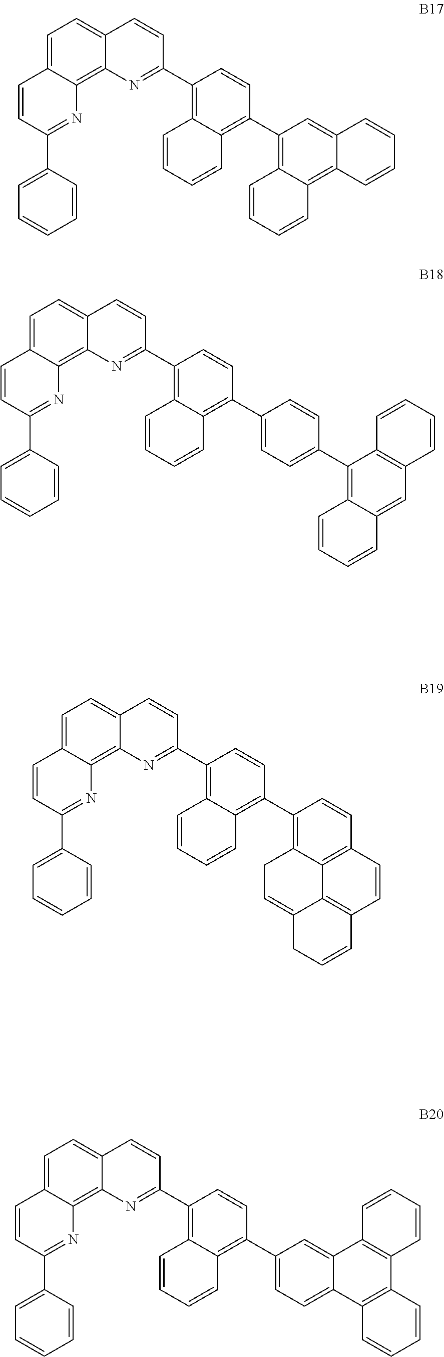 [SMILES: c1ccc(-c2ccc3ccc4ccc(-c5ccc(-c6cc7ccccc7c7ccccc67)c6ccccc56)nc4c3n2)cc1 ; c1ccc(-c2ccc3ccc4ccc(-c5ccc(-c6ccc(-c7c8ccccc8cc8ccccc78)cc6)c6ccccc56)nc4c3n2)cc1 ; C1=Cc2ccc3ccc(-c4ccc(-c5ccc6ccc7ccc(-c8ccccc8)nc7c6n5)c5ccccc45)c4c3c2C(=CC4)C1 ; c1ccc(-c2ccc3ccc4ccc(-c5ccc(-c6ccc7c8ccccc8c8ccccc8c7c6)c6ccccc56)nc4c3n2)cc1]

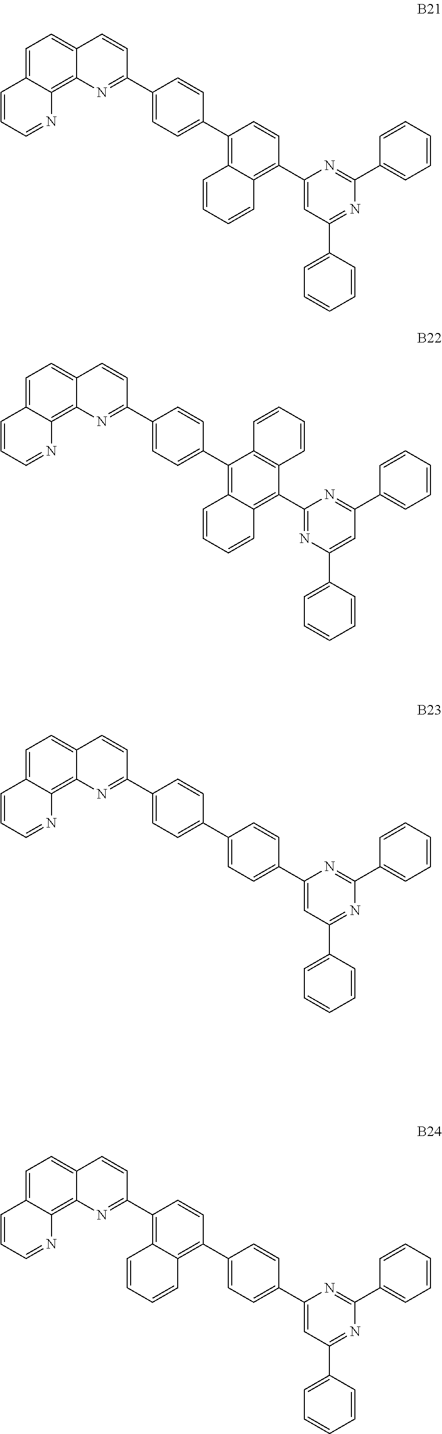 [SMILES: c1ccc(-c2cc(-c3ccc(-c4ccc(-c5ccc6ccc7cccnc7c6n5)cc4)c4ccccc34)nc(-c3ccccc3)n2)cc1 ; c1ccc(-c2cc(-c3ccccc3)nc(-c3c4ccccc4c(-c4ccc(-c5ccc6ccc7cccnc7c6n5)cc4)c4ccccc34)n2)cc1 ; c1ccc(-c2cc(-c3ccc(-c4ccc(-c5ccc6ccc7cccnc7c6n5)cc4)cc3)nc(-c3ccccc3)n2)cc1 ; c1ccc(-c2cc(-c3ccc(-c4ccc(-c5ccc6ccc7cccnc7c6n5)c5ccccc45)cc3)nc(-c3ccccc3)n2)cc1]

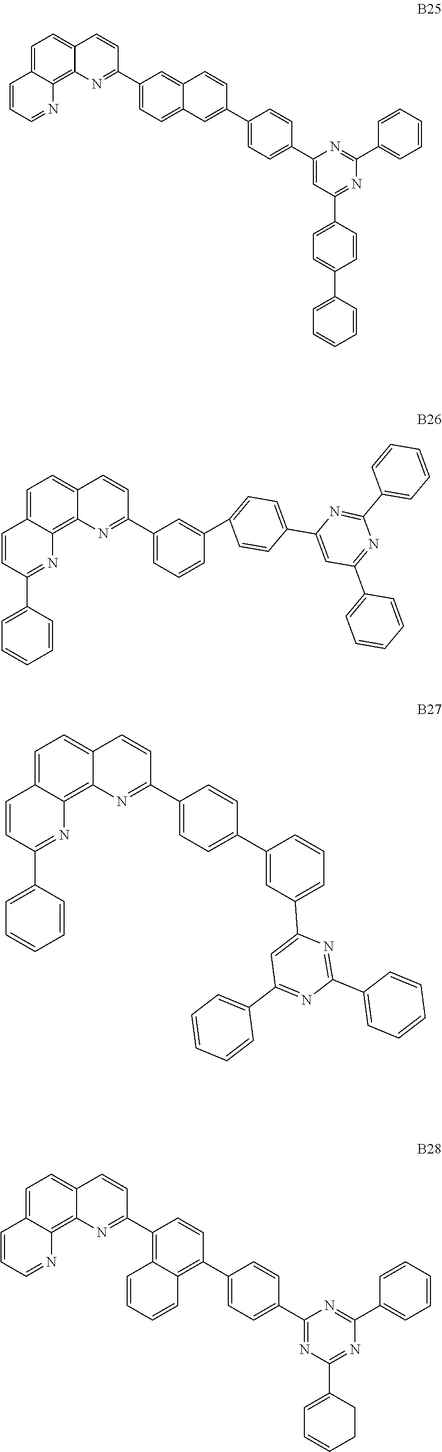 [SMILES: c1ccc(-c2ccc(-c3cc(-c4ccc(-c5ccc6cc(-c7ccc8ccc9cccnc9c8n7)ccc6c5)cc4)nc(-c4ccccc4)n3)cc2)cc1 ; c1ccc(-c2cc(-c3ccc(-c4cccc(-c5ccc6ccc7ccc(-c8ccccc8)nc7c6n5)c4)cc3)nc(-c3ccccc3)n2)cc1 ; c1ccc(-c2cc(-c3cccc(-c4ccc(-c5ccc6ccc7ccc(-c8ccccc8)nc7c6n5)cc4)c3)nc(-c3ccccc3)n2)cc1 ; C1=CCCC(c2nc(-c3ccccc3)nc(-c3ccc(-c4ccc(-c5ccc6ccc7cccnc7c6n5)c5ccccc45)cc3)n2)=C1]

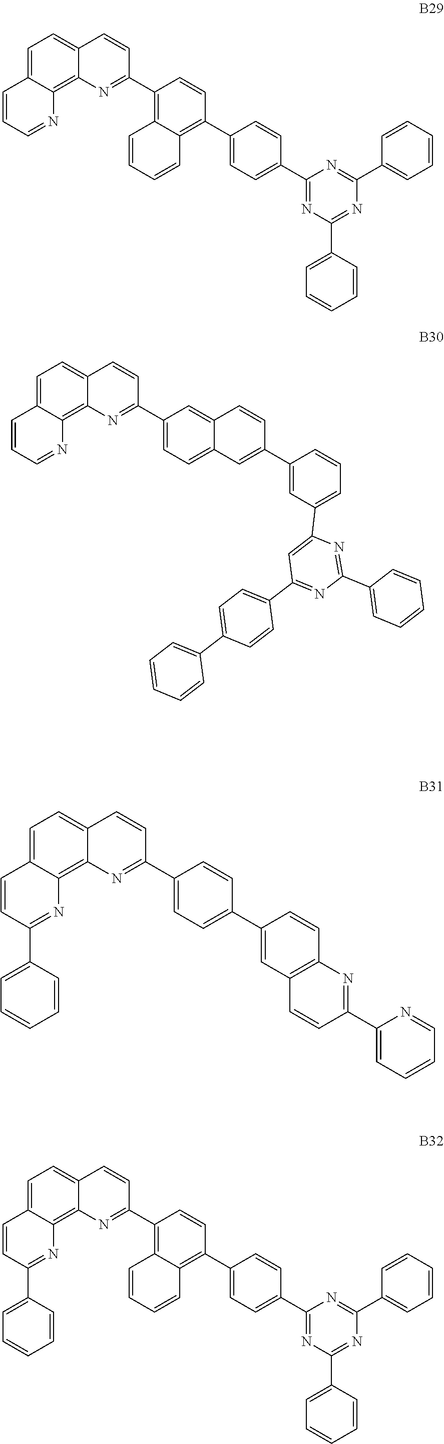 [SMILES: c1ccc(-c2nc(-c3ccccc3)nc(-c3ccc(-c4ccc(-c5ccc6ccc7cccnc7c6n5)c5ccccc45)cc3)n2)cc1 ; c1ccc(-c2ccc(-c3cc(-c4cccc(-c5ccc6cc(-c7ccc8ccc9cccnc9c8n7)ccc6c5)c4)nc(-c4ccccc4)n3)cc2)cc1 ; c1ccc(-c2ccc3ccc4ccc(-c5ccc(-c6ccc7nc(-c8ccccn8)ccc7c6)cc5)nc4c3n2)cc1 ; c1ccc(-c2ccc3ccc4ccc(-c5ccc(-c6ccc(-c7nc(-c8ccccc8)nc(-c8ccccc8)n7)cc6)c6ccccc56)nc4c3n2)cc1]

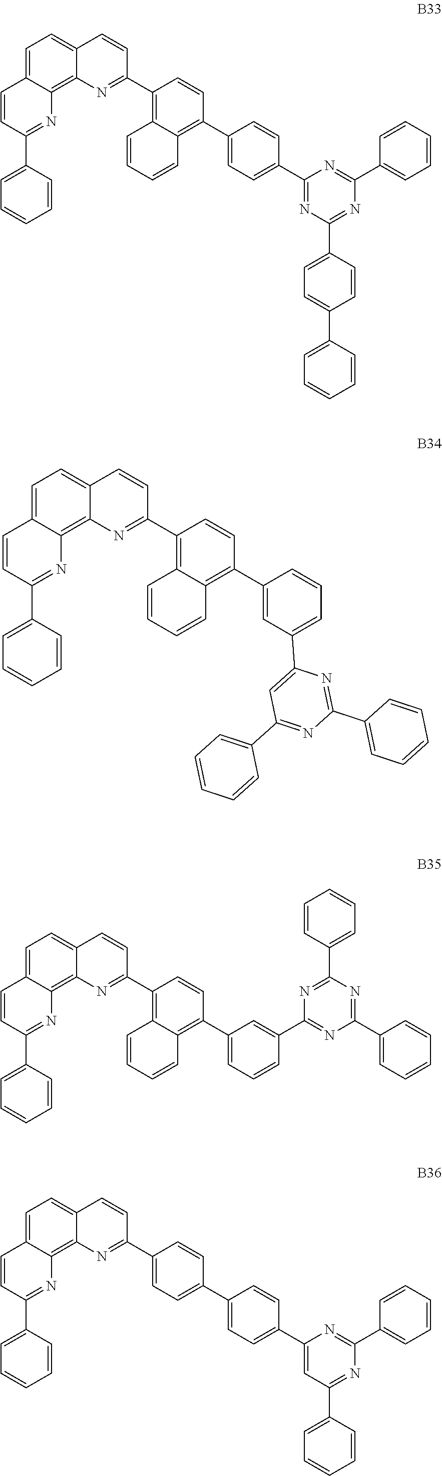 [SMILES: c1ccc(-c2ccc(-c3nc(-c4ccccc4)nc(-c4ccc(-c5ccc(-c6ccc7ccc8ccc(-c9ccccc9)nc8c7n6)c6ccccc56)cc4)n3)cc2)cc1 ; c1ccc(-c2cc(-c3cccc(-c4ccc(-c5ccc6ccc7ccc(-c8ccccc8)nc7c6n5)c5ccccc45)c3)nc(-c3ccccc3)n2)cc1 ; c1ccc(-c2ccc3ccc4ccc(-c5ccc(-c6cccc(-c7nc(-c8ccccc8)nc(-c8ccccc8)n7)c6)c6ccccc56)nc4c3n2)cc1 ; c1ccc(-c2cc(-c3ccc(-c4ccc(-c5ccc6ccc7ccc(-c8ccccc8)nc7c6n5)cc4)cc3)nc(-c3ccccc3)n2)cc1]

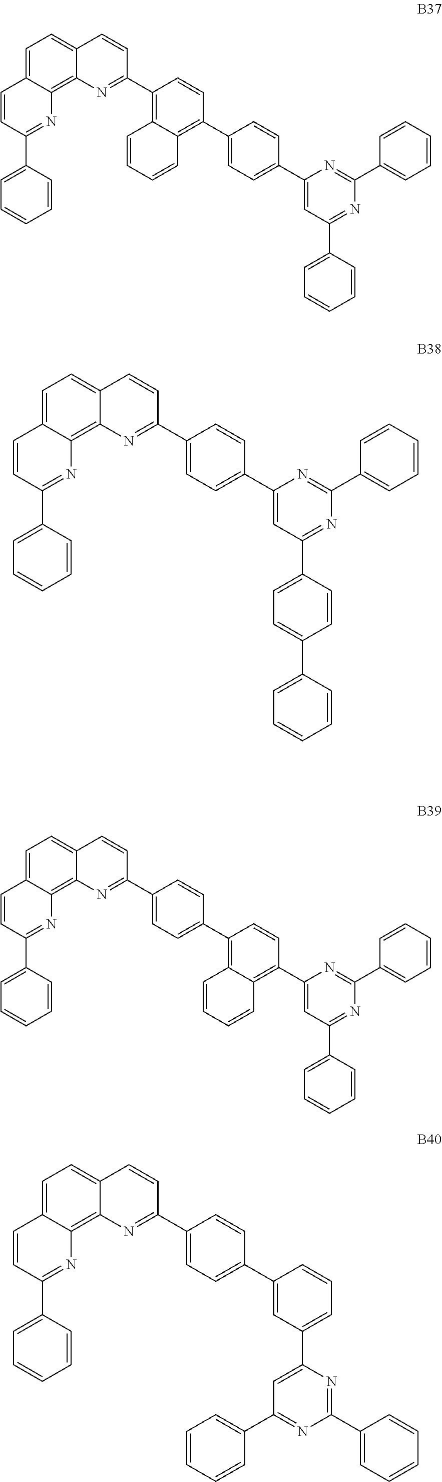 [SMILES: c1ccc(-c2cc(-c3ccc(-c4ccc(-c5ccc6ccc7ccc(-c8ccccc8)nc7c6n5)c5ccccc45)cc3)nc(-c3ccccc3)n2)cc1 ; c1ccc(-c2ccc(-c3cc(-c4ccc(-c5ccc6ccc7ccc(-c8ccccc8)nc7c6n5)cc4)nc(-c4ccccc4)n3)cc2)cc1 ; c1ccc(-c2cc(-c3ccc(-c4ccc(-c5ccc6ccc7ccc(-c8ccccc8)nc7c6n5)cc4)c4ccccc34)nc(-c3ccccc3)n2)cc1 ; c1ccc(-c2cc(-c3cccc(-c4ccc(-c5ccc6ccc7ccc(-c8ccccc8)nc7c6n5)cc4)c3)nc(-c3ccccc3)n2)cc1]

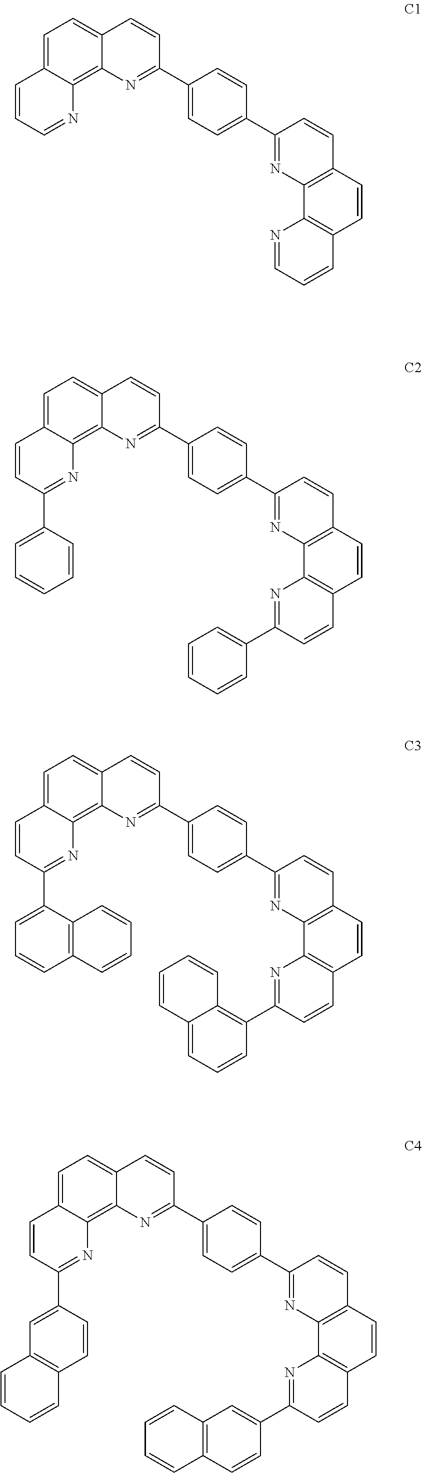 [SMILES: c1cnc2c(c1)ccc1ccc(-c3ccc(-c4ccc5ccc6cccnc6c5n4)cc3)nc12 ; c1ccc(-c2ccc3ccc4ccc(-c5ccc(-c6ccc7ccc8ccc(-c9ccccc9)nc8c7n6)cc5)nc4c3n2)cc1 ; c1ccc2c(-c3ccc4ccc5ccc(-c6ccc(-c7ccc8ccc9ccc(-c%10cccc%11ccccc%10%11)nc9c8n7)cc6)nc5c4n3)cccc2c1 ; c1ccc2cc(-c3ccc4ccc5ccc(-c6ccc(-c7ccc8ccc9ccc(-c%10ccc%11ccccc%11c%10)nc9c8n7)cc6)nc5c4n3)ccc2c1]

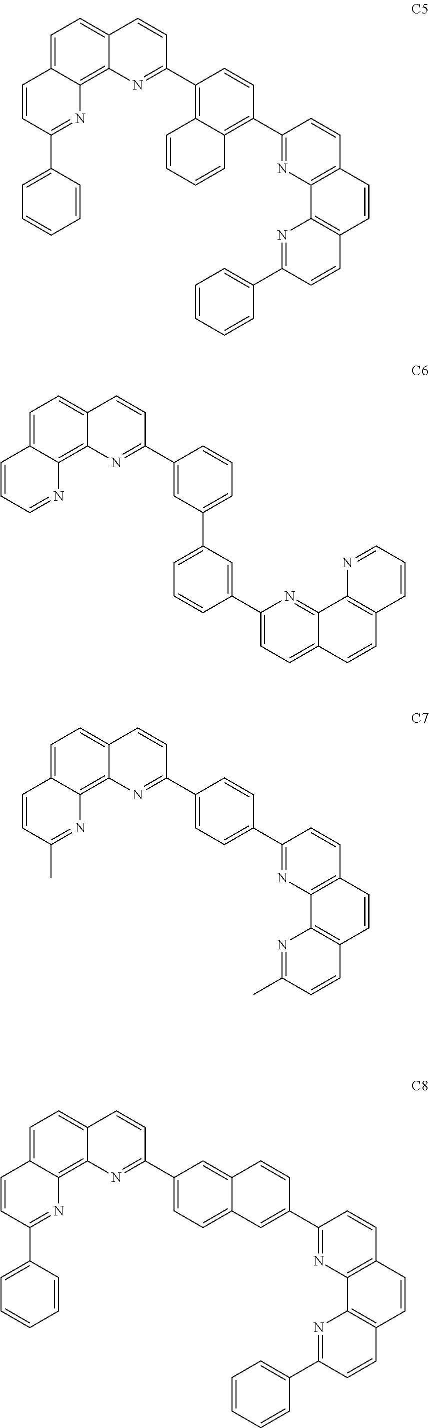 [SMILES: c1ccc(-c2ccc3ccc4ccc(-c5ccc(-c6ccc7ccc8ccc(-c9ccccc9)nc8c7n6)c6ccccc56)nc4c3n2)cc1 ; c1cc(-c2cccc(-c3ccc4ccc5cccnc5c4n3)c2)cc(-c2ccc3ccc4cccnc4c3n2)c1 ; Cc1ccc2ccc3ccc(-c4ccc(-c5ccc6ccc7ccc(C)nc7c6n5)cc4)nc3c2n1 ; c1ccc(-c2ccc3ccc4ccc(-c5ccc6cc(-c7ccc8ccc9ccc(-c%10ccccc%10)nc9c8n7)ccc6c5)nc4c3n2)cc1]

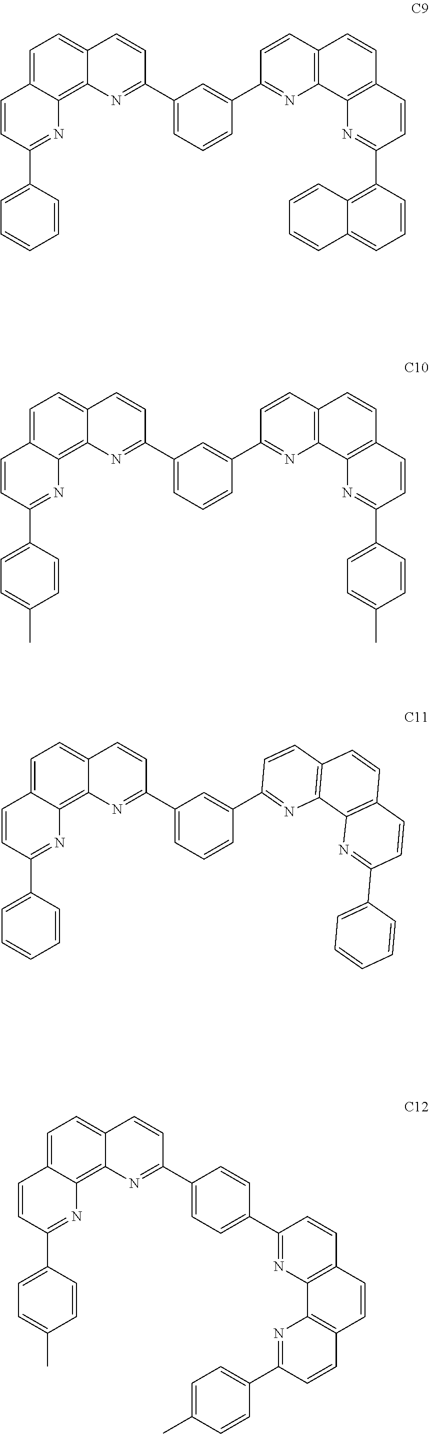 [SMILES: c1ccc(-c2ccc3ccc4ccc(-c5cccc(-c6ccc7ccc8ccc(-c9cccc%10ccccc9%10)nc8c7n6)c5)nc4c3n2)cc1 ; Cc1ccc(-c2ccc3ccc4ccc(-c5cccc(-c6ccc7ccc8ccc(-c9ccc(C)cc9)nc8c7n6)c5)nc4c3n2)cc1 ; c1ccc(-c2ccc3ccc4ccc(-c5cccc(-c6ccc7ccc8ccc(-c9ccccc9)nc8c7n6)c5)nc4c3n2)cc1 ; Cc1ccc(-c2ccc3ccc4ccc(-c5ccc(-c6ccc7ccc8ccc(-c9ccc(C)cc9)nc8c7n6)cc5)nc4c3n2)cc1]

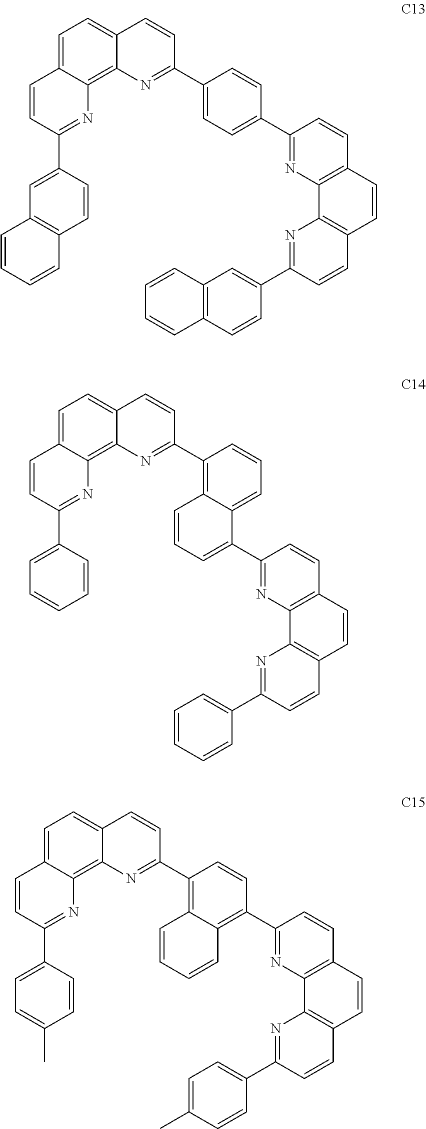 [SMILES: c1ccc2cc(-c3ccc4ccc5ccc(-c6ccc(-c7ccc8ccc9ccc(-c%10ccc%11ccccc%11c%10)nc9c8n7)cc6)nc5c4n3)ccc2c1 ; c1ccc(-c2ccc3ccc4ccc(-c5cccc6c(-c7ccc8ccc9ccc(-c%10ccccc%10)nc9c8n7)cccc56)nc4c3n2)cc1 ; Cc1ccc(-c2ccc3ccc4ccc(-c5ccc(-c6ccc7ccc8ccc(-c9ccc(C)cc9)nc8c7n6)c6ccccc56)nc4c3n2)cc1]

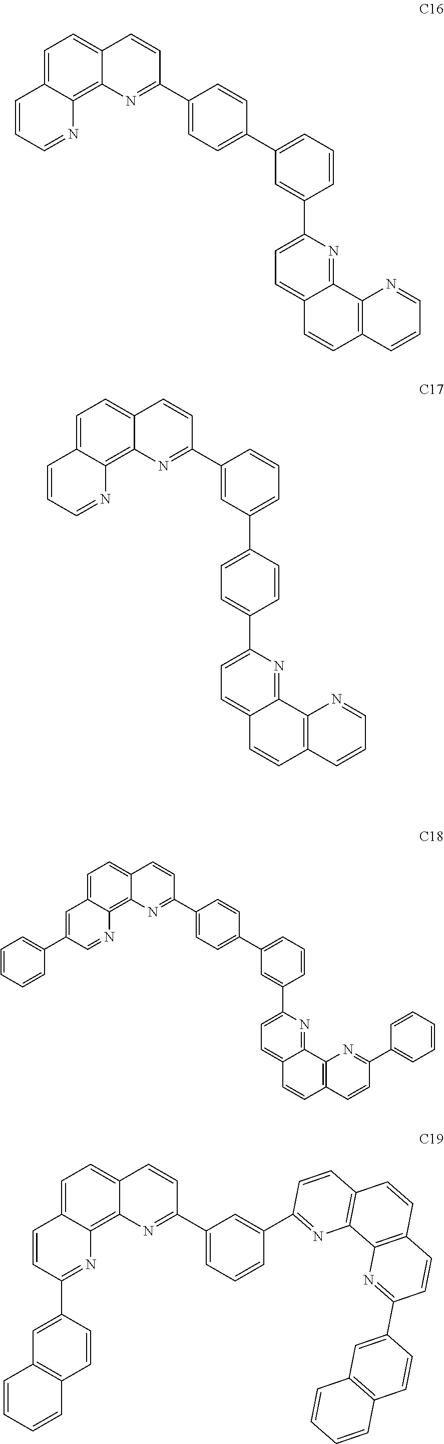 [SMILES: c1cc(-c2ccc(-c3ccc4ccc5cccnc5c4n3)cc2)cc(-c2ccc3ccc4cccnc4c3n2)c1 ; c1cc(-c2ccc(-c3ccc4ccc5cccnc5c4n3)cc2)cc(-c2ccc3ccc4cccnc4c3n2)c1 ; c1ccc(-c2cnc3c(ccc4ccc(-c5ccc(-c6cccc(-c7ccc8ccc9ccc(-c%10ccccc%10)nc9c8n7)c6)cc5)nc43)c2)cc1 ; c1cc(-c2ccc3ccc4ccc(-c5ccc6ccccc6c5)nc4c3n2)cc(-c2ccc3ccc4ccc(-c5ccc6ccccc6c5)nc4c3n2)c1]

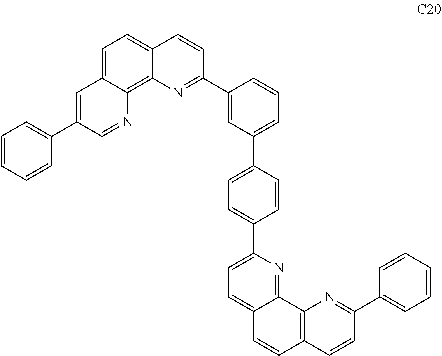 [SMILES: c1ccc(-c2cnc3c(ccc4ccc(-c5cccc(-c6ccc(-c7ccc8ccc9ccc(-c%10ccccc%10)nc9c8n7)cc6)c5)nc43)c2)cc1]

10. The organic light emitting element of claim 1, wherein the organic material layer includes a first light emitting layer and a first layer, and the first layer includes the first compound and the second compound.

11. The organic light emitting element of claim 10, wherein the organic material layer includes a second light emitting layer, and the first layer is positioned between the first light emitting layer and the second light emitting layer.

12. The organic light emitting element of claim 11, wherein the first layer is a charge generation layer.

13. The organic light emitting element of claim 10, wherein the first layer includes a p-type charge generation layer and an n-type charge generation layer, the p-type charge generation layer includes the first compound, and the n-type charge generation layer includes the second compound.

14. The organic light emitting element of claim 13, wherein the first electrode is an anode electrode, the second electrode is a cathode electrode, the n-type charge generation layer is positioned on the first electrode, and the p-type charge generation layer is positioned on the n-type charge generation layer.

15. The organic light emitting element of claim 11, wherein the organic material layer further includes a third light emitting layer and a charge generation layer positioned between the second light emitting layer and the third light emitting layer.

16. A display device comprising the organic light emitting element of claim 1.
